SEQUENCE LISTING (64 sequences)

SEQ ID NO: 1 - protein, 584 aa, synthetic construct
  REGION 1..584: note = Chimeric polypeptide gene switch
  REGION 1..93: note = MISC_FEATURE - "V" region; acidic transcriptional transactivator domain derived from SV40 virus (Simian virus 40); also referred to as a transactivation domain (TAD).
  REGION 94..95: note = MISC_FEATURE - GG (gly-gly) linker
  REGION 96..242: note = MISC_FEATURE - "G" region; DNA-binding domain (DBD)
  REGION 243..249: note = MISC_FEATURE - GGSRRIS (gly-gly-ser-arg-arg-ile-ser) linker
  REGION 250..584: note = MISC_FEATURE - "E" region; mutation substituted ecdysone receptor ligand binding domain Evy (E68V/V184I/Y204E)
MAPPTDVSLGDELHLDGEDVAMAHADALDDFDLDMLGDGDSPGPGFTPHDSAPYGALDMADFEFEQMFTDALGIDEYGGKLLGTSRRISGGEFGGMKLLSSIEQACDICRLKKLKCSKEKPKCAKCLKNNWECRYSPKTKRSPLTRAHLTEVESRLERLEQLFLLIFPREDLDMILKMDSLQDIKALLTGLFVQDNVNKDAVTDRLASVETDMPLTLRQHRISATSSSEESSNKGQRQLTVSGGSRRISRPECVVPETQCAMKRKEKKAQKEKDKLPVSTTTVDDHMPPIMQCEPPPPEAARIHEVVPRFLSDKLLVTNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSILFANNQAYTRDNYRKAGMAEVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 2 - protein, 335 aa, synthetic construct
  REGION 1..335: note = Polypeptide Gene Switch
  SITE 68: note = MISC_FEATURE - Position at which E (Glu) in wild-type ecdysone receptor ligand binding domain is substituted with V (Val).
  SITE 184: note = MISC_FEATURE - Position at which V (Val) in wild-type ecdysone receptor ligand binding domain is substituted with I (Ile).
  SITE 204: note = MISC_FEATURE - Position at which Y (Tyr) in wild-type ecdysone receptor ligand binding domain is substituted with E (Glu).
RPECVVPETQCAMKRKEKKAQKEKDKLPVSTTTVDDHMPPIMQCEPPPPEAARIHEVVPRFLSDKLLVTNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSILFANNQAYTRDNYRKAGMAEVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 3 - DNA, 13176 bp, synthetic construct
  misc_feature 1..13176: note = Polynucleotide Vector
  misc_feature 223..774: note = Complement Phosphinothricin N-acetyltransferase
  misc_feature 792..1090: note = Complement NOS Promoter
  misc_feature 1123..1146: note = attB1
  misc_feature 1177..1471: note = Complement E9 Terminator
  misc_feature 1478..2005: note = Complement At-FT
  misc_feature 2019..2236: note = Complement RS Promoter
  misc_feature 2252..2812: note = Complement 3A Terminator
  misc_feature 2819..4579: note = VGEvy (E68V/V184I/Y204E)
  misc_feature 4594..5242: note = Complement35S Promoter
  misc_feature 5797..6483: note = Resolvase
  source 1..13176: mol_type = other DNA
taactataacggtcctaaggtagcgacgtacgaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagttagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatttttgctccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttggcgcgccatcacaagtttgtacaaaaaagcaggctccgaattcgcccttggcgcgcctcggaccggtttgggatgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatatcgatctaaagtcttcttcctccgcagccactctccctctgacaattgtagaaaactgcggccacgggaaggccgagattgtagatctcagcaaactcgcgagtgttgaagttctggcgccaccctggtgcatacactgtttgcctgccaagctgtcgaaacaatataaacacgacacgatgaattcctgcagtgggacttggattttcgtaacacacaatctcattgccaaaggttgttccagttgtagcagggatatcagtcaccaaccaatggagatattctcggaggtgagggttgctaggacttggaacatctggatccaccataaccaaagtatagaagttcctgaggtcttctccaccaatctcaactcttggcttgttttgaacctgagaaggccttagatccaagccattagtcacctctctttggccataagtaacctttagagtgattgatctattaaacggatcaagaacgtctccaacaactctgcttactataagagggtctcttatatttatagacatctttgcctgcaggaccggaatgccaagctggaattcgagctcctgcagctcgaagatccaagcttcgtgtcctctccaaatgaaatgaacttccttatatagaggaagggtcttgctctagtgtctccgctcggaggacagtactccgctcggaggacagtactccgctcggaggacagtactccgctcggaggacagtactccgctcggaggacagtactccgagaattcagcggccgcctcgagctcagctagaataattatcagacaaatattgagattctggtatatcaagaaaaaatgttctattttggtggtaagagagattcatcaagtccaataaaaactacaaacatgatttgaaaattgcagaaaggaaagtgaataaatgttgacacaaaaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaacatcgattcagagattcgtgggggactcaaggataggcggcggttgggtgtgcgacatgtccgccacatcccaaatctcctcaaggaaaggcggcagctttctgttcttgagcttgagggagatgcacatgttggagttttgcattccgagcgtgcgtagctcagagaggattgagaggatcttgccgtatatgacggacgaacgcgccgaaccggaaagttggttcaggatatagatgcggagcgtattcaggtagtaccgctggatttcctccaccagttgcggctgctccaaccctggccggtcagaaaagatgacgacagccgtgagcagcgcgtaatggatgttgtccaacgccatagagtacatgcaccggcagaagtgcagtagatcctcgatgacttcggccatgccagccttgcggtagttatccctggtgtacgcttggttgttagcgaacagaatactgtcggaggccgcatcgtatcgtcgcgcgactcggagcatcattacctcacttgagcaagccttgagcagcgtaatttgatctggctggctgatcttggcgaaccctggcaatcccttagcgaactccacgataagttggaccgtgaggatagtcatctccgtgatctggcggaagggagtgtccgattcttcgttttcatcgtccgcttgctgccacgtctgcgtaatccttttaagatcctcatcagaaggctgctcgtacccgtcttgataccagatgagcctggcgataaggaactgctggttggctgtcaactgggggatgtttttctgccggtttgtcaccaacagcttgtcggagagaaaccttgggacaacctcgtgaatccttgctgcttcaggaggtggaggttcacactgcataatgggcggcatgtggtcgtccaccgtcgtcgtgctgacaggcagtttgtccttctccttctgtgctttcttctctttccgcttcatggcgcactgagtctcgggtactacgcactcaggccgtgatattctcctagacccgcccgatacagtcaactgtctttgacctttgttactactctcctccgatgatgatgtcgcacttattctatgctgtctcaatgttagaggcatatcagtctccactgaagccaatctatctgtgacggcatccttgttcacattatcttgtacaaataatccggtaagaagtgcttttatatcctgtaaagaatccattttcaaaatcatgtcaaggtcctcccttggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcaggcttgttcgatagaagaaagtagcttcatcccgccgaactccccgcctgatattcttctggaggttccaagcaacttcccaccgtactcgtcaattccaagggcatcggtaaacatctgctcaaactcgaagtcggccatatccagagcgccgtagggggcggagtcgtggggggtaaatcccggacctggggaatccccgtcccccaacatgtccagatcgaaatcgtctagcgcgtcagcgtgagccatcgccacgtcctcgccgtctaagtggagttcgtcccccaggctgacatcggtcgggggggccatggtggcgctagccctgcaggtctggaggatctgctagagtcagcttgtcgcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccacgcgtcacagttgaggtatttcggatcgtggcgatcgcaagggcgaattcgacccagctttcttgtacaaagtggtgatcggtccggttcggctgcggccgcattaccctgttatccctatagatctttaactatgactctcttaaggtagccaaattccggaacgcgccgaagtttaaacgacttaatcagctaatacaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggctgctgctgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagctgctgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtaccaagaaggccaagaacggccgcctggtgacggtatccgagggtgaagccttgattagccgctacaagatcgtaaagagcgaaaccgggcggccggagtacatcgagatcgaactggctgattggatgtaccgcgagatcacagaaggcaagaacccggacgtgctgacggttcaccccgattactttttgatcgaccccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcacggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtgctgatgaatcccctaatgatttttatcaaaatcattaagttaaggtagatacacatcttgtcatatgatcaaatggtttcgccaaaaatcaataatcagacaacaaaatgtgcgaactcgatattttacacgactctctttaccaattctgccccgaattacacttaaaacgactcaacagcttaacgttggcttgccacgccttacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatatccgtgagcaaaaacggcttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatggattctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagactcactcgtggctgcaaccctgcttactcaaaatctattcacacatggttacgttttggggaaattatgaggggatctctcagcgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaatgtcgac

SEQ ID NO: 4 - DNA, 10601 bp, synthetic construct
  misc_feature 1..10601: note = Polynucleotide Vector
  misc_feature 33..222: note = Complement CaMV 35S Terminator
  misc_feature 223..774: note = Complement Bar
  misc_feature 792..1090: note = Complement NOS Promoter
  misc_feature 1177..1471: note = Complement E9 Terminator
  misc_feature 2720..2743: note = Complement attB2
  misc_feature 3222..3908: note = Resolvase
  source 1..10601: mol_type = other DNA
taactataacggtcctaaggtagcgacgtacgaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagttagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcatttttgctccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttggcgcgccatcacaagtttgtacaaaaaagcaggctccgaattcgcccttggcgcgcctcggaccggtttgggatgttttactcctcatattaacttcggtcattagaggccacgatttgacacatttttactcaaaacaaaatgtttgcatatctcttataatttcaaattcaacacacaacaaataagagaaaaaacaaataatattaatttgagaatgaacaaaaggaccatatcattcattaactcttctccatccatttccatttcacagttcgatagcgaaaaccgaataaaaaacacagtaaattacaagcacaacaaatggtacaagaaaaacagttttcccaatgccataatatcgatctaaagtcttcttcctccgcagccactctccctctgacaattgtagaaaactgcggccacgggaaggccgagattgtagatctcagcaaactcgcgagtgttgaagttctggcgccaccctggtgcatacactgtttgcctgccaagctgtcgaaacaatataaacacgacacgatgaattcctgcagtgggacttggattttcgtaacacacaatctcattgccaaaggttgttccagttgtagcagggatatcagtcaccaaccaatggagatattctcggaggtgagggttgctaggacttggaacatctggatccaccataaccaaagtatagaagttcctgaggtcttctccaccaatctcaactcttggcttgttttgaacctgagaaggccttagatccaagccattagtcacctctctttggccataagtaacctttagagtgattgatctattaaacggatcaagaacgtctccaacaactctgcttactataagagggtctcttatatttatagacatctttgcctgcaggtctggaggatctgctagagtcagcttgtcgcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccacgcgtcacagttgaggtatttcggatcgtggcgatcgcaagggcgaattcgacccagctttcttgtacaaagtggtgatcggtccggttcggctgcggccgcattaccctgttatccctatagatctttaactatgactctcttaaggtagccaaattccggaacgcgccgaagtttaaacgacttaatcagctaatacaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggctgctgctgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagctgctgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtaccaagaaggccaagaacggccgcctggtgacggtatccgagggtgaagccttgattagccgctacaagatcgtaaagagcgaaaccgggcggccggagtacatcgagatcgaactggctgattggatgtaccgcgagatcacagaaggcaagaacccggacgtgctgacggttcaccccgattactttttgatcgaccccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcacggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtgctgatgaatcccctaatgatttttatcaaaatcattaagttaaggtagatacacatcttgtcatatgatcaaatggtttcgccaaaaatcaataatcagacaacaaaatgtgcgaactcgatattttacacgactctctttaccaattctgccccgaattacacttaaaacgactcaacagcttaacgttggcttgccacgccttacttgactgtaaaactctcactcttaccgaacttggccgtaacctgccaaccaaagcgagaacaaaacataacatcaaacgaatcgaccgattgttaggtaatcgtcacctccacaaagagcgactcgctgtataccgttggcatgctagctttatctgttcgggcaatacgatgcccattgtacttgttgactggtctgatatccgtgagcaaaaacggcttatggtattgcgagcttcagtcgcactacacggtcgttctgttactctttatgagaaagcgttcccgctttcagagcaatgttcaaagaaagctcatgaccaatttctagccgaccttgcgagcattctaccgagtaacaccacaccgctcattgtcagtgatgctggctttaaagtgccatggtataaatccgttgagaagctgggttggtactggttaagtcgagtaagaggaaaagtacaatatgcagacctaggagcggaaaactggaaacctatcagcaacttacatgatatgtcatctagtcactcaaagactttaggctataagaggctgactaaaagcaatccaatctcatgccaaattctattgtataaatctcgctctaaaggccgaaaaaatcagcgctcgacacggactcattgtcaccacccgtcacctaaaatctactcagcgtcggcaaaggagccatggattctagcaactaacttacctgttgaaattcgaacacccaaacaacttgttaatatctattcgaagcgaatgcagattgaagaaaccttccgagacttgaaaagtcctgcctacggactaggcctacgccatagccgaacgagcagctcagagcgttttgatatcatgctgctaatcgccctgatgcttcaactaacatgttggcttgcgggcgttcatgctcagaaacaaggttgggacaagcacttccaggctaacacagtcagaaatcgaaacgtactctcaacagttcgcttaggcatggaagttttgcggcattctggctacacaataacaagggaagactcactcgtggctgcaaccctgcttactcaaaatctattcacacatggttacgttttggggaaattatgaggggatctctcagcgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaatgtcgac

SEQ ID NO: 5 - DNA, 552 bp, Streptomyces hygroscopicus
  source 1..552: mol_type = unassigned DNA
ttagatctcggtgacgggcaggaccggacggggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggctcat

SEQ ID NO: 6 - DNA, 299 bp, Agrobacterium tumefaciens
  source 1..299: mol_type = unassigned DNA
tgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatc

SEQ ID NO: 7 - DNA, 24 bp, Escherichia coli
  source 1..24: mol_type = unassigned DNA
caagtttgtacaaaaaagcaggct

SEQ ID NO: 8 - DNA, 528 bp, Arabidopsis thaliana
  source 1..528: mol_type = unassigned DNA
ctaaagtcttcttcctccgcagccactctccctctgacaattgtagaaaactgcggccacgggaaggccgagattgtagatctcagcaaactcgcgagtgttgaagttctggcgccaccctggtgcatacactgtttgcctgccaagctgtcgaaacaatataaacacgacacgatgaattcctgcagtgggacttggattttcgtaacacacaatctcattgccaaaggttgttccagttgtagcagggatatcagtcaccaaccaatggagatattctcggaggtgagggttgctaggacttggaacatctggatccaccataaccaaagtatagaagttcctgaggtcttctccaccaatctcaactcttggcttgttttgaacctgagaaggccttagatccaagccattagtcacctctctttggccataagtaacctttagagtgattgatctattaaacggatcaagaacgtctccaacaactctgcttactataagagggtctcttatatttatagacat

SEQ ID NO: 9 - DNA, 51 bp, Cauliflower mosaic virus
  source 1..51: mol_type = unassigned DNA
aacttccttatatagaggaagggtcttgcgaaggatagtgggattgtgcgt

SEQ ID NO: 10 - DNA, 93 bp, Saccharomyces cerevisiae
  source 1..93: mol_type = unassigned DNA
tggagatatcacatcaatccacttgctttgaagacgtggttggaacgtcttctttttccacgatgctcctcgtgggtgggggtccatctttgg

SEQ ID NO: 11 - DNA, 1761 bp, synthetic construct
  misc_feature 1..1761: note = Encodes a Chimeric Sequence Forming Monopartite Gene Switch Polypeptide
  source 1..1761: mol_type = other DNA
gccaaattccggaacgcgccgaagtttaaacgacttaatcagctaatacaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggctgctgctgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagc

SEQ ID NO: 12 - DNA, 24 bp, Escherichia coli
  source 1..24: mol_type = unassigned DNA
caaatcggcgcggcgctgggtgat

SEQ ID NO: 13 - DNA, 2507 bp, Arabidopsis thaliana
  source 1..2507: mol_type = unassigned DNA
acaaattaaagaagcagaaacaaaaacaagtaaaacagaaacaatcaacacagagaaaccacctgtttgttcaagatcaaagatgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttctttcacttgaactcccttttgtctcttttcttctttagtttcttcagtgcttcttacaccttcttttttaaaatagaaattattttccttttttggggtatactgaaaatatttcttgggcatgcagagaccttggttaaaaatgccccacgctttccttttctctgtttttttatgatttatttggttttactttatgatacccaaatcaaaaactaatttatatttcatttcttttttcatgaagatggacccataaaatatttcttcctatcctaaattaaatagagataaatttatgatctatcccaaattttttccaccaacttcttgcataagtgattattatttaggataagaatcttgggatttttctttgttcctcctacctaataatttaatttgcatttgtttttttttgtttttttttaagtataattttttactatactttgaaaatggcatttttgaaatatcctttttgctaattaaaatatctgcaaaaagatatagaaaattgatgttcattaaaacaaatatatatattgatgaatctctgttgtggaatatttgaaaactgtttgaataaggatattctgatattcaagccagcctttaagatactctctgctatatatagacatgtagctactaccttttttctattcatagttttcttcttctttcttgtgttatctcattttccaaacttcaaaaaagaaaaagaaaaaaagaccttttgctttcttgatttctttgaaaatgataatcttatcttcttataattcttcgtcttatttgtttaatgaaggttatggtggatccagatgttccaagtcctagcaaccctcacctccgagaatatctccattggtttgtgcactaactcaactctttaattaattcacttttaagttatagcatagctcaaacatgttgctcgaattatatatatagacactcaactacacatgtaaaactggacatgtattttggatactttgaaattgagtagatcacttataacttaagactcaaacattttacatttaatagaaggagacatataaaatcaacttcgagagtgcgatgcatcaatttgtctcccaaaaaagcccacacccaagctaaattgacatattttgttcaaaaactttactatgtgtaatgtaaatatgtattatgtatgtctgtgtattttactgatcgcacatagattctatagaatgtataggacgtgacttgaactaagatttgtttttttcactttaaagtggtctttgtacgggaatatcattgagcttaaaatagcctgatgacatcatcattgttccttgaagtgagcaggactaggtttcctgttatatcactttttatttttatttatatattcatgcacttgaatgttattgcatgttttgctagtcacttgcactagtaatctaggaattagttacgttgctattatttttttgtaagaaaataaagtttaagtagcatttcaattctgtcaaaaaaagaagtagcatttcaattatgttgtggtgccatagcttaaacatgtgtatggctatctctcattacaggttggtgactgatatccctgctacaactggaacaacctttggtgagttttattctatatattagatcgctaggtgttagaaatatagaaaggtatatgaaatagcctaattaattagttactagctagaaaattcacatgttttgatgaactttttatttttcaggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttagatggcttcttcctttataaccaattgatattgcatactctgatgagatttatgcatctatagtattttaatttaataaccattttatgatacgagtaacgaacggtgatgatgcctatagtagttcaatatataagtgtgtaataaaaatgagagggggaggaaaatgagagtgttttacttatatagtgtgtgatgcgataattatattaatctacatgaaatgaagtgttatatttatacttt

SEQ ID NO: 14 - DNA, 855 bp, Arabidopsis thaliana
  source 1..855: mol_type = unassigned DNA
acaaattaaagaagcagaaacaaaaacaagtaaaacagaaacaatcaacacagagaaaccacctgtttgttcaagatcaaagatgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttagatggcttcttcctttataaccaattgatattgcatactctgatgagatttatgcatctatagtattttaatttaataaccattttatgatacgagtaacgaacggtgatgatgcctatagtagttcaatatataagtgtgtaataaaaatgagagggggaggaaaatgagagtgttttacttatatagtgtgtgatgcgataattatattaatctacatgaaatgaagtgttatatttatacttt

SEQ ID NO: 15 - DNA, 528 bp, Arabidopsis thaliana
  source 1..528: mol_type = unassigned DNA
atgtctataaatataagagaccctcttatagtaagcagagttgttggagacgttcttgatccgtttaatagatcaatcactctaaaggttacttatggccaaagagaggtgactaatggcttggatctaaggccttctcaggttcaaaacaagccaagagttgagattggtggagaagacctcaggaacttctatactttggttatggtggatccagatgttccaagtcctagcaaccctcacctccgagaatatctccattggttggtgactgatatccctgctacaactggaacaacctttggcaatgagattgtgtgttacgaaaatccaagtcccactgcaggaattcatcgtgtcgtgtttatattgtttcgacagcttggcaggcaaacagtgtatgcaccagggtggcgccagaacttcaacactcgcgagtttgctgagatctacaatctcggccttcccgtggccgcagttttctacaattgtcagagggagagtggctgcggaggaagaagactttag

SEQ ID NO: 16 - DNA, 1056 bp, Arabidopsis thaliana
  source 1..1056: mol_type = unassigned DNA
atggcggcgatttcaggcatctcctctggtacgttgacgatttcacggcctttggttactcttcgacgctctagagccgccgtttcgtacagctcctctcaccgattgcttcatcatcttcctctctcttctcgtcgtctgctattaaggaacaatcatcgagtccaagcaacgattttgcaagacgatgaagagaaagtggtggtggaggaatcgtttaaagccgagacttctactggtacagaaccacttgaggagccaaatatgagttcttcttcaactagtgcttttgagacatggatcatcaagcttgagcaaggagtgaatgttttccttacagactcggttattaagatacttgacactttgtatcgtgaccgaacatatgcaaggttctttgttcttgagacaattgctagagtgccttattttgcgtttatgtctgtgctacatatgtatgagacctttggttggtggaggagagcagattatttgaaagtacactttgctgagagctggaatgaaatgcatcacttgctcataatggaagaattgggtggaaattcttggtggtttgatcgttttctggctcagcacatagcaaccttctactacttcatgacagtgttcttgtatatcttaagccctagaatggcatatcacttttcggaatgtgtggagagtcatgcatatgagacttatgataaatttctcaaggccagtggagaggagttgaagaatatgcctgcaccggatatcgcagtaaaatactatacgggaggtgacttgtacttatttgatgagttccaaacatcaagaactcccaatactcgaagaccagtaatagaaaatctatacgatgtgtttgtgaacataagagatgatgaagcagaacactgcaagacaatgagagcttgtcagactctaggcagtctgcgttctccacactccattttagaagatgatgatactgaagaagaatcagggtgtgttgttcctgaggaggctcattgcgaaggtattgtagactgcctcaagaaatccattacaagttaa

SEQ ID NO: 17 - protein, 149 aa, Bacillus amyloliquefaciens
MKKRLSWISVCLLVLVSAAGMLFSTAAKTETSSHKAHTEAQVINTFDGVADYLQTYHKLPDNYITKSEAQALGWVASKGNLADVAPGKSIGGDIFSNREGKLPGKSGRTWREADINYTSGFRNSDRILYSSDWLIYKTTDHYQTFTKIR

SEQ ID NO: 18 - protein, 101 aa, Bacillus amyloliquefaciens
MEIVIIDGKDVTSTEALHRILKDQLDFPDFYGENLNALWDCLTGWIEYPLTLVWKNFEISQKELGSDADDVLELFQEAQAELDGEFFIQIDQPSAGTTDRH

SEQ ID NO: 19 - protein, 513 aa, Choristoneura fumiferana
MDLKHEVAYRGVLPGQVKAEPGVHNGQVNGHVRDWMAGGAGANSPSPGAVAQPQPNNGYSSPLSSGSYGPYSPNGKIGREELSPASSINGCSTDGEARRQKKGPAPRQQEELCLVCGDRASGYHYNALTCEGCKGFFRRSVTKNAVYICKFGHACEMDMYMRRKCQECRLKKCLAVGMRPECVVPETQCAMKRKEKKAQKEKDKLPVSTTTVDDHMPPIMQCEPPPPEAARIHEVVPRFLSDKLLETNRQKNIPQLTANQQFLIARLIWYQDGYEQPSDEDLKRITQTWQQADDENEESDTPFRQITEMTILTVQLIVEFAKGLPGFAKISQPDQITLLKACSSEVMMLRVARRYDAASDSVLFANNQAYTRDNYRKAGMAYVIEDLLHFCRCMYSMALDNIHYALLTAVVIFSDRPGLEQPQLVEEIQRYYLNTLRIYILNQLSGSARSSVIYGKILSILSELRTLGMQNSNMCISLKLKNRKLPPFLEEIWDVADMSHTQPPPILESPTNL

SEQ ID NO: 20 - protein, 879 aa, Drosophila melanogaster
MKRRWSNNGGFMRLPEESSSEVTSSSNGLVLPSGVNMSPSSLDSHDYCDQDLWLCGNESGSFGGSNGHGLSQQQQSVITLAMHGCSSTLPAQTTIIPINGNANGNGGSTNGQYVPGATNLGALANGMLNGGFNGMQQQIQNGHGLINSTTPSTPTTPLHLQQNLGGAGGGGIGGMGILHHANGTPNGLIGVVGGGGGVGLGVGGGGVGGLGEQHTPRSDSVNSISSGRDDLSPSSSLNGYSANESCDAKKSKKGPAPRVQEELCLVCGDRASGYHYNALTCEGCKGFFRRSVTKSAVYCCKFGRACEMDMYMRRKCQECRLKKCLAVGGERPECVVPENQCAMKRREKKAQKEKDKMTTSPSSQHGGNGSLASGGGQDFVKKEILDLMTCEPPQHATIPLLPDEILAKCQARNIPSLTYNQLAVIYKLIWYQDGYEQPSEEDLRRIMSQPDENESQTDVSFRHITEITILTVQLIVEFAKGLPAFTKIPQEDQITLLKACSSEVMMLRMARRYDHSSDSIFFANNRSYTRDSYKMAGMADNIEDLLHFCRQMFSMKVDNVEYALLTAIVIFSDRPGLEKAQLVEAIQSYYIDTLRIYILNRHCGDSMSLVFYAKLLSILTELRTLGNQNAEMCFSLKLKNRKLPKFLEEIWDVHAIPPSVQSHLQITQEENERLERAERMRASVGGAITAGIDCDSASTSAAAAAAQHQPQPQPQPQPSSLTQNDSQHQTQPQLQPQLPPQLQGQLQPQLQPQLQTQLQPQIQPQPQLLPVSAPVPASVTAPGSLSAVSTSSEYMGGSAAIGPITPATTSSITAAVTASSTTSAVPMGNGVGVGVGVGGNVSMYANAQTAMALMGVALHSHQEQLIGGVAVKSEHSTTA

SEQ ID NO: 21 - DNA, 24 bp, synthetic construct
  misc_feature 1..24: note = Oligonucleotide Primer
  source 1..24: mol_type = other DNA
catctggaggttgtgaactaatgg

SEQ ID NO: 22 - DNA, 23 bp, synthetic construct
  misc_feature 1..23: note = Oligonucleotide Primer
  source 1..23: mol_type = other DNA
ttgttcttgtcttaagcgcttga

SEQ ID NO: 23 - DNA, 18 bp, synthetic construct
  misc_feature 1..18: note = Oligonucleotide Primer
  misc_feature 1: note = Conjugated to VIC(R) reporter dye
  misc_feature 18: note = Conjugated to minor groove binder non-fluorescent quencher (MGBNFQ)
  source 1..18: mol_type = other DNA
acatagctttagggttcc

SEQ ID NO: 24 - DNA, 23 bp, synthetic construct
  misc_feature 1..23: note = Oligonucleotide Primer
  source 1..23: mol_type = other DNA
ttatggccaaagagaggtgacta

SEQ ID NO: 25 - DNA, 23 bp, synthetic construct
  misc_feature 1..23: note = Oligonucleotide Primer
  source 1..23: mol_type = other DNA
ccaatctcaactcttggcttgtt

SEQ ID NO: 26 - DNA, 24 bp, synthetic construct
  misc_feature 1..24: note = Oligonucleotide Primer
  misc_feature 1..24: note = Conjugated to internal ZEN(R) Quencher
  misc_feature 1: note = Conjugated to 6-carboxyfluorescein (FAM(R)) Reporter dye
  misc_feature 24: note = Conjugated to Iowa Black FQ (IBFQ) Quencher
  source 1..24: mol_type = other DNA
tggcttggatctaaggccttctca

SEQ ID NO: 27 - DNA, 22 bp, synthetic construct
  misc_feature 1..22: note = Oligonucleotide Primer
  source 1..22: mol_type = other DNA
gatgtgatatctccactgacgt

SEQ ID NO: 28 - DNA, 23 bp, synthetic construct
  misc_feature 1..23: note = Oligonucleotide Primer
  source 1..23: mol_type = other DNA
cgtgtcctctccaaatgaaatga

SEQ ID NO: 29 - DNA, 24 bp, synthetic construct
  misc_feature 1..24: note = Oligonucleotide Primer
  misc_feature 1..24: note = Conjugated to internal ZEN(R) Quencher
  misc_feature 1: note = Conjugated to 6-carboxyfluorescein (FAM(R)) Reporter dye
  misc_feature 24: note = Conjugated to Iowa Black FQ (IBFQ) Quencher
  source 1..24: mol_type = other DNA
cgcacaatcccactatccttcgca

SEQ ID NO: 30 - DNA, 18 bp, synthetic construct
  misc_feature 1..18: note = Oligonucleotide Primer
  source 1..18: mol_type = other DNA
gaggtcgtccgtccactc

SEQ ID NO: 31 - DNA, 23 bp, synthetic construct
  misc_feature 1..23: note = Oligonucleotide Primer
  source 1..23: mol_type = other DNA
gtcaaccactacatcgagacaag

SEQ ID NO: 32 - DNA, 20 bp, synthetic construct
  misc_feature 1..20: note = Oligonucleotide Primer
  misc_feature 1..20: note = Conjugated to internal ZEN(R) Quencher
  misc_feature 1: note = Conjugated to 6-carboxyfluorescein (FAM(R)) Reporter dye
  misc_feature 20: note = Conjugated to Iowa Black FQ (IBFQ) Quencher
  source 1..20: mol_type = other DNA
cggttcctgcggctcggtac

SEQ ID NO: 33 - DNA, 21 bp, synthetic construct
  misc_feature 1..21: note = Oligonucleotide Primer
  source 1..21: mol_type = other DNA
tgtttgggaatgtttctgcgg

SEQ ID NO: 34 - DNA, 22 bp, synthetic construct
  misc_feature 1..22: note = Oligonucleotide Primer
  source 1..22: mol_type = other DNA
cttcaagcaacagaggtttgtg

SEQ ID NO: 35 - DNA, 2442 bp, synthetic construct
  source 1..2442: mol_type = other DNA
tgcaggactcttgagtactttcgccagctcacgttgacttttttcgctatcactcctttttagtgtcaaaacgtaggggtcttcgaaaatgttgagactttgcacaatctttctcaggctcgccacagttttcgtcacgatgatatttgactctggggtatagtagataatagatagaagtggtagctttactcgaaggcgaagatctgctcgatgtatttttggcgtgcagcaaatggcatccgtccagatctggtcaacacaccaagatcctagagttctactggcttctgtcccaaatacaaatggcttccgaaagataggattgtcttttagaagcgggagcatttgaagataaagaggagtattgaatttcgggcccaagggatcatagtaggcaagagtctcaggtttacctttgattgagtactgcagcaagctgggatcttctgcagtacatatttgactgtgtagcaaaccttcaagttcttccgcatcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctggtatacctgccttgtggtaagcctccttcagtccttcccctttcaaaggtcataaacgctaaagcactactcaaaatttattactcatttttgaggaatcgtgacaaaccataaaacattgacgaaagatctaataatatcatatttttacaatcttttcacctcgtgaacacctattgtataaaaagacagatatattggtttttatttaagttattttacatgaaataagcattttaattttgtgattgtgcagatgtgtttaaccacgtggtataccagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgatgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggacggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagtcctgcaggatcgatgacttcccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggggct

SEQ ID NO: 36 - protein, 175 aa, Arabidopsis thaliana
MSINIRDPLIVSRVVGDVLDPFNRSITLKVTYGQREVTNGLDLRPSQVQNKPRVEIGGEDLRNFYTLVMVDPDVPSPSNPHLREYLHWLVTDIPATTGTTFGNEIVCYENPSPTAGIHRVVFILFRQLGRQTVYAPGWRQNFNTREFAEIYNLGLPVAAVFYNCQRESGCGGRRL

SEQ ID NO: 37 - protein, 9 aa, Arabidopsis thaliana
TELRLGLPG

SEQ ID NO: 38 - protein, 9 aa, Arabidopsis thaliana
TELRLGLPE

SEQ ID NO: 39 - protein, 9 aa, Arabidopsis thaliana
TELCLGLPG

SEQ ID NO: 40 - protein, 9 aa, Arabidopsis thaliana
TELTLGLPG

SEQ ID NO: 41 - protein, 9 aa, Arabidopsis thaliana
TELTLALPG

SEQ ID NO: 42 - protein, 9 aa, Arabidopsis thaliana
TDLRLGLSF

SEQ ID NO: 43 - protein, 9 aa, Arabidopsis thaliana
TELDLALGL

SEQ ID NO: 44 - protein, 9 aa, Arabidopsis thaliana
SELELGLGL

SEQ ID NO: 45 - protein, 9 aa, Arabidopsis thaliana
MELDLGLSL

SEQ ID NO: 46 - protein, 9 aa, Arabidopsis thaliana
IELGLTLSL

SEQ ID NO: 47 - protein, 9 aa, Arabidopsis thaliana
IDLGLDLRT

SEQ ID NO: 48 - protein, 9 aa, Arabidopsis thaliana
VNLSLSLTF

SEQ ID NO: 49 - protein, 9 aa, Arabidopsis thaliana
KKLELKLGP

SEQ ID NO: 50 - protein, 9 aa, Arabidopsis thaliana
KKLELRLHR

SEQ ID NO: 51 - protein, 9 aa, Arabidopsis thaliana
KRLELRLAP

SEQ ID NO: 52 - protein, 9 aa, Nicotiana tabacum
IDLDLNLAP

SEQ ID NO: 53 - protein, 9 aa, Arabidopsis thaliana
LDLELNLPP

SEQ ID NO: 54 - protein, 9 aa, Arabidopsis thaliana
QDLDLELRL

SEQ ID NO: 55 - DNA, 14669 bp, synthetic construct
  misc_feature 1..14669: note = ID164 Vector
  source 1..14669: mol_type = other DNA
taactataacggtcctaaggtagcgacgtacgtgcggagggacactatcaatcgtagctcgagtttgatatccaaaatagacgagaacaataagcaaaaactcttagttttgaaataaatcaacaatcccgagggttgtcacatatacatcaaaaacgaaaatccatatagcaaaaaaaaactctaaattaccgttcgacaaaaaagaaaactgataagacatttgctaaacattaaaaatcgattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatacctatcagccacaccaagccttccacagtcgatgaatccagaaaagcggccattttccaccatgatattcggcaagcaggcatcgccatgggtcacgacgagatcctcgccgtctggcatcctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagcagcgcaaggaacgcccgtcgtggccagccacgatagccgcgctgcctcgtcttgaagttcattcagggcaccggacaggtcggtcttgacaaaaagaactggccttccctgcgctgacagccggaacacggcggcatcagagcagccgattgtctgttgtgcccagtcatagccgaatagcctctccacccaagcggccggagaacctgcgtgcaatccatcttgttcaatcatgtttaaactaccaccacggagacggagcacgagatggagagttgactccttctggatgttgtagtcggcaagagtacgaccatcctcaagctgctttccggcgaaaatcaaacgctgctggtctgggggaatcccttccttgtcctggatcttggctttgacattgtcgatggtgtcggaagactcaacctctagggtgatcgtcttccccgttaaggtcttcacgaagatctgcatttttgcgctagcccgaaaagcacacaatgccctgcaggcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgacgcgtcatgagaagttcactcgtgacgaagggcgcgccatcacaagtttgtacaaaaaagcaggctccgaattcgcccttggcgcgcctcggaccgcgttgctcacgttacagctccattcctcgagccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgggaagtcatcgatatcctgcagggcaacactgtggaggacaacagtctcaactgttagcccaggcccaaatccaaaaagaacaccccactcaagcccttcaccagtagttcctaaaccttctttggctgaggcctttctcatttcatccaaaataaacagtacacaagcacttgacatgttaccatagtcacttaacacattccttgtagccttaagtttctcgggctttaggcccaactttatttcaacttggtccaaaattgcaggcccacctggatgagcaatccagaatagagagttccaatcagaaatgcccaaaggtttaaatgcttcctcaaggctcttctcaatattttttgagatcagcccaggaacatctttgagtaagtggaatgtaagcccaacttcacggagatggccatcaatagcaccatggctatctgggagaagagtttgggctgctgaaacgagctcgaacaaaggcctctcgactcctggaattggatcagaacctataatgatcgcgcctgccccatcaccaaaaagggcttggccaactaaactatccacacgtggttaaacacatctgcacaatcacaaaattaaaatgcttatttcatgtaaaataacttaaataaaaaccaatatatctgtctttttatacaataggtgttcacgaggtgaaaagattgtaaaaatatgatattattagatctttcgtcaatgttttatggtttgtcacgattcctcaaaaatgagtaataaattttgagtagtgctttagcgtttatgacctttgaaaggggaaggactgaaggaggcttaccacaaggcaggtagtgtggatagtttagttggccaagccctttttggtgatggggcaggcgcgatcattataggttctgatccaattccaggagtcgagaggcctttgttcgagctcgtttcagcagcccaaactcttctcccagatagccatggtgctattgatggccatctccgtgaagttgggcttacattccacttactcaaagatgttcctgggctgatctcaaaaaatattgagaagagccttgaggaagcatttaaacctttgggcatttctgattggaactctctattctggattgctcatccaggtgggcctgcaattttggaccaagttgaaataaagttgggcctaaagcccgagaaacttaaggctacaaggaatgtgttaagtgactatggtaacatgtcaagtgcttgtgtactgtttattttggatgaaatgagaaaggcctcagccaaagaaggtttaggaactactggtgaagggcttgagtggggtgttctttttggatttgggcctgggctaacagttgagactgttgtcctccacagtgttgccctgcaggaccggaatgccaagctggaattcgagctcctgcagctcgaagatccaagcttcgtgtcctctccaaatgaaatgaacttccttatatagaggaagggtcttgctctagtgtctccgctcggaggacagtactccgctcggaggacagtactccgctcggaggacagtactccgctcggaggacagtactccgctcggaggacagtactccgagaattcagcggccgcctcgagctcagctagaataattatcagacaaatattgagattctggtatatcaagaaaaaatgttctattttggtggtaagagagattcatcaagtccaataaaaactacaaacatgatttgaaaattgcagaaaggaaagtgaataaatgttgacacaaaaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaacatcgattcagagattcgtgggggactcaaggataggcggcggttgggtgtgcgacatgtccgccacatcccaaatctcctcaaggaaaggcggcagctttctgttcttgagcttgagggagatgcacatgttggagttttgcattccgagcgtgcgtagctcagagaggattgagaggatcttgccgtatatgacggacgaacgcgccgaaccggaaagttggttcaggatatagatgcggagcgtattcaggtagtaccgctggatttcctccaccagttgcggctgctccaaccctggccggtcagaaaagatgacgacagccgtgagcagcgcgtaatggatgttgtccaacgccatagagtacatgcaccggcagaagtgcagtagatcctcgatgacttcggccatgccagccttgcggtagttatccctggtgtacgcttggttgttagcgaacagaatactgtcggaggccgcatcgtatcgtcgcgcgactcggagcatcattacctcacttgagcaagccttgagcagcgtaatttgatctggctggctgatcttggcgaaccctggcaatcccttagcgaactccacgataagttggaccgtgaggatagtcatctccgtgatctggcggaagggagtgtccgattcttcgttttcatcgtccgcttgctgccacgtctgcgtaatccttttaagatcctcatcagaaggctgctcgtacccgtcttgataccagatgagcctggcgataaggaactgctggttggctgtcaactgggggatgtttttctgccggtttgtcaccaacagcttgtcggagagaaaccttgggacaacctcgtgaatccttgctgcttcaggaggtggaggttcacactgcataatgggcggcatgtggtcgtccaccgtcgtcgtgctgacaggcagtttgtccttctccttctgtgctttcttctctttccgcttcatggcgcactgagtctcgggtactacgcactcaggccgtgatattctcctagacccgcccgatacagtcaactgtctttgacctttgttactactctcctccgatgatgatgtcgcacttattctatgctgtctcaatgttagaggcatatcagtctccactgaagccaatctatctgtgacggcatccttgttcacattatcttgtacaaataatccggtaagaagtgcttttatatcctgtaaagaatccattttcaaaatcatgtcaaggtcctcccttggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcaggcttgttcgatagaagaaagtagcttcatcccgccgaactccccgcctgatattcttctggaggttccaagcaacttcccaccgtactcgtcaattccaagggcatcggtaaacatctgctcaaactcgaagtcggccatatccagagcgccgtagggggcggagtcgtggggggtaaatcccggacctggggaatccccgtcccccaacatgtccagatcgaaatcgtctagcgcgtcagcgtgagccatcgccacgtcctcgccgtctaagtggagttcgtcccccaggctgacatcggtcgggggggccatggtggcgctagccctgcaggtctggaggatctgctagagtcagcttgtcgcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccacgcgtcacagttgaggtatttcggatcgtggcgatcgcaagggcgaattcgacccagctttcttgtacaaagtggtgatcggtccggttcggctgcggccgcattaccctgttatccctaatctcgtttaactatgactctcttaaggtagccaaattccggaaaacgacttaatcagctaatacaaactatcagtgtttgacaggatatattggcgggtaaacctaagagaaaagagcgtttattagaataatcggatatttaaaagggcgtgaaaaggtttatccgttcgtccatttgtatgtgcatgcggctggggcgagcaccagacggagcaccgccaggatcacgagccatatcctgacgaatgggttctcgtccaacgcccctttatcgacagtatgtgaatttgcagtgtggccgcgatcaacgcacactggccttgggtctgaagggtgatcttcaaaacgaacccccgtcttggtggtcagtgtgatgggcacatcgcttactcctcagtcaccctttgagccagataattaagaggcaaatgcaattggctcaggctgccatcgtccccccgtgcgaaacctgcacgtccgcgtcaaagaaataaccggcacctcttgctgtttttatcagttgagggcttgacggatcagcctcaagtttgcggcgcagcctcaaaatgagaacatctatactcctgtcataaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgctagaaactctctgatactgaacggcttagcgataaaatcacttgctcctagctcaagtgcaacaactttatccgtctcctcaaggcggtcgccactgataattatgattggaatatcagactttgccgccagattacgaacgatctcaagcccatcttcacgacctaaatctagatcaacaaccacgacatcgaccgtcgcggaagagagtactcttgtgaactgggtgctgtcggctaccgcggtcactttgaaggcgtggatcgtaaggtattcaataataagatgccgcatagcgacgtcgtcatcgacaagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacaaaaatgcgaaccaagtatttcaattttatgacaaaaattctcaatcgttgttacaagtgaaacgcttcgaggttacagctactattgatttaggagatcgcctatggtctcgccccggcgtcgtgcgtccgccgcgagccgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggctgctgctgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgctggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttctttaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagctgctgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtaccaagaaggccaagaacggccgcctggtgacggtatccgagggtgaagccttgattagccgctacaagatcgtaaagagcgaaaccgggcggccggagtacatcgagatcgaactggctgattggatgtaccgcgagatcacagaaggcaagaacccggacgtgctgacggttcaccccgattactttttgatcgaccccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcacggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaatgtcgac

SEQ ID NO: 56 - protein, 389 aa, Petunia axillaris
MVTVEEYRKAQRAEGPATVMAIGTATPTNCVDQSTYPDYYFRITNSEHKTDLKEKFKRMCEKSMIKKRYMHLTEEILKENPSMCEYMAPSLDARQDIVVVEVPKLGKEAAQKAIKEWGQPKSKITHLFFCTTSGVDMPGCDYQLTKLLGLRPSVKRLMMYQQGCFAGGTVLRLAKDLAENNKGARVLVVCSEITAVTFRGPNDTHLDSLVGQALFGDGAGAIIIGSDPIPGVERPLFELVSAAQTLLPDSHGAIDGHLREVGLTFHLLKDVPGLISKNIEKSLEEAFKPLGISDWNSLFWIAHPGGPAILDQVEIKLGLKPEKLKATRNVLSDYGNMSSACVLFILDEMRKASAKEGLGTTGEGLEWGVLFGFGPGLTVETVVLHSVAT

SEQ ID NO: 57 - DNA, 15457 bp, synthetic construct
  misc_feature 1..15457: note = Vector ID163
  source 1..15457: mol_type = other DNA
taactataacggtcctaaggtagcgacgtacgtgcggagggacactatcaatcgtagctcgagtttgatatccaaaatagacgagaacaataagcaaaaactcttagttttgaaataaatcaacaatcccgagggttgtcacatatacatcaaaaacgaaaatccatatagcaaaaaaaaactctaaattaccgttcgacaaaaaagaaaactgataagacatttgctaaacattaaaaatcgattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatacctatcagccacaccaagccttccacagtcgatgaatccagaaaagcggccattttccaccatgatattcggcaagcaggcatcgccatgggtcacgacgagatcctcgccgtctggcatcctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagcagcgcaaggaacgcccgtcgtggccagccacgatagccgcgctgcctcgtcttgaagttcattcagggcaccggacaggtcggtcttgacaaaaagaactggccttccctgcgctgacagccggaacacggcggcatcagagcagccgattgtctgttgtgcccagtcatagccgaatagcctctccacccaagcggccggagaacctgcgtgcaatccatcttgttcaatcatgtttaaactaccaccacggagacggagcacgagatggagagttgactccttctggatgttgtagtcggcaagagtacgaccatcctcaagctgctttccggcgaaaatcaaacgctgctggtctgggggaatcccttccttgtcctggatcttggctttgacattgtcgatggtgtcggaagactcaacctctagggtgatcgtcttccccgttaaggtcttcacgaagatctgcatttttgcgctagcccgaaaagcacacaatgccctgcaggcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgacgcgtcatgagaagttcactcgtgacgaagggcgcgccatcacaagtttgtacaaaaaagcaggctccgaattcgcccttggcgcgcctcggaccgcgttgctcacgttacagctccattcctcgagccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgggaagtcatcgatacctgcaggactcttgagtactttcgccagctcacgttgacttttttcgctatcactcctttttagtgtcaaaacgtaggggtcttcgaaaatgttgagactttgcacaatctttctcaggctcgccacagttttcgtcacgatgatatttgactctggggtatagtagataatagatagaagtggtagctttactcgaaggcgaagatctgctcgatgtatttttggcgtgcagcaaatggcatccgtccagatctggtcaacacaccaagatcctagagttctactggcttctgtcccaaatacaaatggcttccgaaagataggattgtcttttagaagcgggagcatttgaagataaagaggagtattgaatttcgggcccaagggatcatagtaggcaagagtctcaggtttacctttgattgagtactgcagcaagctgggatcttctgcagtacatatttgactgtgtagcaaaccttcaagttcttccgcatcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctggtatacacacgtggttaaacacatctgcacaatcacaaaattaaaatgcttatttcatgtaaaataacttaaataaaaaccaatatatctgtctttttatacaataggtgttcacgaggtgaaaagattgtaaaaatatgatattattagatctttcgtcaatgttttatggtttgtcacgattcctcaaaaatgagtaataaattttgagtagtgctttagcgtttatgacctttgaaaggggaaggactgaaggaggcttaccacaaggcaggtataccagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgatgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggacggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagtcctgcaggaccggaatgccaagctggaattcgagctcctgcagctcgaagatccaagcttcgtgtcctctccaaatgaaatgaacttccttatatagaggaagggtcttgctctagtgtctccgctcggaggacagtactccgctcggaggacagtactccgctcggaggacagtactccgctcggaggacagtactccgctcggaggacagtactccgagaattcagcggccgcctcgagctcagctagaataattatcagacaaatattgagattctggtatatcaagaaaaaatgttctattttggtggtaagagagattcatcaagtccaataaaaactacaaacatgatttgaaaattgcagaaaggaaagtgaataaatgttgacacaaaaagcctatactgtacttaacttgattgcataattacttgatcatagactcatagtaaacttgattacacagataagtgaagaaacaaaccaattcaagacataaccaaagagaggtgaaagactgttttatatgtctaacattgcaccttaatatcacactgttagttcctttcttacttaaattcaacccattaaagtaaaaacaacagataataataatttgagaatgaacaaaaggaccatatcatttattaactcttatccatccatttgcattttgatgtccgaaaacaaaaactgaaagaacacagtaaattacaagcagaacaaatgatagaagaaaacagcttttccaatgccataatactcaaacttagtaggattctggtgtgtgggcaatgaaacatcgattcagagattcgtgggggactcaaggataggcggcggttgggtgtgcgacatgtccgccacatcccaaatctcctcaaggaaaggcggcagctttctgttcttgagcttgagggagatgcacatgttggagttttgcattccgagcgtgcgtagctcagagaggattgagaggatcttgccgtatatgacggacgaacgcgccgaaccggaaagttggttcaggatatagatgcggagcgtattcaggtagtaccgctggatttcctccaccagttgcggctgctccaaccctggccggtcagaaaagatgacgacagccgtgagcagcgcgtaatggatgttgtccaacgccatagagtacatgcaccggcagaagtgcagtagatcctcgatgacttcggccatgccagccttgcggtagttatccctggtgtacgcttggttgttagcgaacagaatactgtcggaggccgcatcgtatcgtcgcgcgactcggagcatcattacctcacttgagcaagccttgagcagcgtaatttgatctggctggctgatcttggcgaaccctggcaatcccttagcgaactccacgataagttggaccgtgaggatagtcatctccgtgatctggcggaagggagtgtccgattcttcgttttcatcgtccgcttgctgccacgtctgcgtaatccttttaagatcctcatcagaaggctgctcgtacccgtcttgataccagatgagcctggcgataaggaactgctggttggctgtcaactgggggatgtttttctgccggtttgtcaccaacagcttgtcggagagaaaccttgggacaacctcgtgaatccttgctgcttcaggaggtggaggttcacactgcataatgggcggcatgtggtcgtccaccgtcgtcgtgctgacaggcagtttgtccttctccttctgtgctttcttctctttccgcttcatggcgcactgagtctcgggtactacgcactcaggccgtgatattctcctagacccgcccgatacagtcaactgtctttgacctttgttactactctcctccgatgatgatgtcgcacttattctatgctgtctcaatgttagaggcatatcagtctccactgaagccaatctatctgtgacggcatccttgttcacattatcttgtacaaataatccggtaagaagtgcttttatatcctgtaaagaatccattttcaaaatcatgtcaaggtcctcccttggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcaggcttgttcgatagaagaaagtagcttcatcccgccgaactccccgcctgatattcttctggaggttccaagcaacttcccaccgtactcgtcaattccaagggcatcggtaaacatctgctcaaactcgaagtcggccatatccagagcgccgtagggggcggagtcgtggggggtaaatcccggacctggggaatccccgtcccccaacatgtccagatcgaaatcgtctagcgcgtcagcgtgagccatcgccacgtcctcgccgtctaagtggagttcgtcccccaggctgacatcggtcgggggggccatggtggcgctagccctgcaggtctggaggatctgctagagtcagcttgtcgcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccacgcgtcacagttgaggtatttcggatcgtggcgatcgcaagggcgaattcgacccagctttcttgtacaaagtggtgatcggtccggttcggctgcggccgcattaccctgttatccctaatctcgtttaactatgactctcttaaggtagccaaattccggaaaacgacttaatcagctaatacaaactatcagtgtttgacaggatatattggcgggtaaacctaagagaaaagagcgtttattagaataatcggatatttaaaagggcgtgaaaaggtttatccgttcgtccatttgtatgtgcatgcggctggggcgagcaccagacggagcaccgccaggatcacgagccatatcctgacgaatgggttctcgtccaacgcccctttatcgacagtatgtgaatttgcagtgtggccgcgatcaacgcacactggccttgggtctgaagggtgatcttcaaaacgaacccccgtcttggtggtcagtgtgatgggcacatcgcttactcctcagtcaccctttgagccagataattaagaggcaaatgcaattggctcaggctgccatcgtccccccgtgcgaaacctgcacgtccgcgtcaaagaaataaccggcacctcttgctgtttttatcagttgagggcttgacggatcagcctcaagtttgcggcgcagcctcaaaatgagaacatctatactcctgtcataaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgctagaaactctctgatactgaacggcttagcgataaaatcacttgctcctagctcaagtgcaacaactttatccgtctcctcaaggcggtcgccactgataattatgattggaatatcagactttgccgccagattacgaacgatctcaagcccatcttcacgacctaaatctagatcaacaaccacgacatcgaccgtcgcggaagagagtactcttgtgaactgggtgctgtcggctaccgcggtcactttgaaggcgtggatcgtaaggtattcaataataagatgccgcatagcgacgtcgtcatcgacaagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacaaaaatgcgaaccaagtatttcaattttatgacaaaaattctcaatcgttgttacaagtgaaacgcttcgaggttacagctactattgatttaggagatcgcctatggtctcgccccggcgtcgtgcgtccgccgcgagccgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggctgctgctgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgctggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttctttaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagctgctgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtaccaagaaggccaagaacggccgcctggtgacggtatccgagggtgaagccttgattagccgctacaagatcgtaaagagcgaaaccgggcggccggagtacatcgagatcgaactggctgattggatgtaccgcgagatcacagaaggcaagaacccggacgtgctgacggttcaccccgattactttttgatcgaccccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcacggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaatgtcgac

SEQ ID NO: 58 - protein, 602 aa, Escherichia coli
MLRPVETPTREIKKLDGLWAFSLDRENCGIDQRWWESALQESRAIAVPGSFNDQFADADIRNYAGNVWYQREVFIPKGWAGQRIVLRFDAVTHYGKVWVNNQEVMEHQGGYTPFEADVTPYVIAGKSVRITVCVNNELNWQTIPPGMVITDENGKKKQSYFHDFFNYAGIHRSVMLYTTPNTWVDDITVVTHVAQDCNHASVDWQVVANGDVSVELRDADQQVVATGQGTSGTLQVVNPHLWQPGEGYLYELCVTAKSQTECDIYPLRVGIRSVAVKGQQFLINHKPFYFTGFGRHEDADLRGKGFDNVLMVHDHALMDWIGANSYRTSHYPYAEEMLDWADEHGIVVIDETAAVGFNLSLGIGFEAGNKPKELYSEEAVNGETQQAHLQAIKELIARDKNHPSVVMWSIANEPDTRPQVHGNISPLAEATRKLDPTRPITCVNVMFCDAHTDTISDLFDVLCLNRYYGWYVQSGDLETAEKVLEKELLAWQEKLHQPIIITEYGVDTLAGLHSMYTDMWSEEYQCAWLDMYHRVFDRVSAVVGEQVWNFADFATSQGILRVGGNKKGIFTRDRKPKSAAFLLQKRWTGMNFGEKPQQGGKQ

SEQ ID NO: 59 - DNA, 12143 bp, synthetic construct
  misc_feature 1..12143: note = Polynucleotide Vector
  source 1..12143: mol_type = other DNA
taactataacggtcctaaggtagcgacgtacgtgcggagggacactatcaatcgtagctcgagtttgatatccaaaatagacgagaacaataagcaaaaactcttagttttgaaataaatcaacaatcccgagggttgtcacatatacatcaaaaacgaaaatccatatagcaaaaaaaaactctaaattaccgttcgacaaaaaagaaaactgataagacatttgctaaacattaaaaatcgattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatacctatcagccacaccaagccttccacagtcgatgaatccagaaaagcggccattttccaccatgatattcggcaagcaggcatcgccatgggtcacgacgagatcctcgccgtctggcatcctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagcagcgcaaggaacgcccgtcgtggccagccacgatagccgcgctgcctcgtcttgaagttcattcagggcaccggacaggtcggtcttgacaaaaagaactggccttccctgcgctgacagccggaacacggcggcatcagagcagccgattgtctgttgtgcccagtcatagccgaatagcctctccacccaagcggccggagaacctgcgtgcaatccatcttgttcaatcatgtttaaactaccaccacggagacggagcacgagatggagagttgactccttctggatgttgtagtcggcaagagtacgaccatcctcaagctgctttccggcgaaaatcaaacgctgctggtctgggggaatcccttccttgtcctggatcttggctttgacattgtcgatggtgtcggaagactcaacctctagggtgatcgtcttccccgttaaggtcttcacgaagatctgcatttttgcgctagcccgaaaagcacacaatgccctgcaggcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgacgcgtcatgagaagttcactcgtgacgaagggcgcgccatcacaagtttgtacaaaaaagcaggctccgaattcgcccttgcgatcgccggaccgctcgagcccgggcggccgcagccgaaccggaccgcgttgctcacgttacagctccattcctcgagccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgggaagtcatcgatatcctgcagggcaacactgtggaggacaacagtctcaactgttagcccaggcccaaatccaaaaagaacaccccactcaagcccttcaccagtagttcctaaaccttctttggctgaggcctttctcatttcatccaaaataaacagtacacaagcacttgacatgttaccatagtcacttaacacattccttgtagccttaagtttctcgggctttaggcccaactttatttcaacttggtccaaaattgcaggcccacctggatgagcaatccagaatagagagttccaatcagaaatgcccaaaggtttaaatgcttcctcaaggctcttctcaatattttttgagatcagcccaggaacatctttgagtaagtggaatgtaagcccaacttcacggagatggccatcaatagcaccatggctatctgggagaagagtttgggctgctgaaacgagctcgaacaaaggcctctcgactcctggaattggatcagaacctataatgatcgcgcctgccccatcaccaaaaagggcttggccaactaaactatccacacgtggttaaacacatctgcacaatcacaaaattaaaatgcttatttcatgtaaaataacttaaataaaaaccaatatatctgtctttttatacaataggtgttcacgaggtgaaaagattgtaaaaatatgatattattagatctttcgtcaatgttttatggtttgtcacgattcctcaaaaatgagtaataaattttgagtagtgctttagcgtttatgacctttgaaaggggaaggactgaaggaggcttaccacaaggcaggtagtgtggatagtttagttggccaagccctttttggtgatggggcaggcgcgatcattataggttctgatccaattccaggagtcgagaggcctttgttcgagctcgtttcagcagcccaaactcttctcccagatagccatggtgctattgatggccatctccgtgaagttgggcttacattccacttactcaaagatgttcctgggctgatctcaaaaaatattgagaagagccttgaggaagcatttaaacctttgggcatttctgattggaactctctattctggattgctcatccaggtgggcctgcaattttggaccaagttgaaataaagttgggcctaaagcccgagaaacttaaggctacaaggaatgtgttaagtgactatggtaacatgtcaagtgcttgtgtactgtttattttggatgaaatgagaaaggcctcagccaaagaaggtttaggaactactggtgaagggcttgagtggggtgttctttttggatttgggcctgggctaacagttgagactgttgtcctccacagtgttgccctgcaggtctggaggatctgctagagtcagcttgtcgcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccacgcgtcacagttgaggtatttcggatcgtggcgatcgctatccccaggcgcgccaagggcgaattcgacccagctttcttgtacaaagtggtgatcggtccggttcggctgcggccgcattaccctgttatccctaatctcgtttaactatgactctcttaaggtagccaaattccggaaaacgacttaatcagctaatacaaactatcagtgtttgacaggatatattggcgggtaaacctaagagaaaagagcgtttattagaataatcggatatttaaaagggcgtgaaaaggtttatccgttcgtccatttgtatgtgcatgcggctggggcgagcaccagacggagcaccgccaggatcacgagccatatcctgacgaatgggttctcgtccaacgcccctttatcgacagtatgtgaatttgcagtgtggccgcgatcaacgcacactggccttgggtctgaagggtgatcttcaaaacgaacccccgtcttggtggtcagtgtgatgggcacatcgcttactcctcagtcaccctttgagccagataattaagaggcaaatgcaattggctcaggctgccatcgtccccccgtgcgaaacctgcacgtccgcgtcaaagaaataaccggcacctcttgctgtttttatcagttgagggcttgacggatcagcctcaagtttgcggcgcagcctcaaaatgagaacatctatactcctgtcataaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgctagaaactctctgatactgaacggcttagcgataaaatcacttgctcctagctcaagtgcaacaactttatccgtctcctcaaggcggtcgccactgataattatgattggaatatcagactttgccgccagattacgaacgatctcaagcccatcttcacgacctaaatctagatcaacaaccacgacatcgaccgtcgcggaagagagtactcttgtgaactgggtgctgtcggctaccgcggtcactttgaaggcgtggatcgtaaggtattcaataataagatgccgcatagcgacgtcgtcatcgacaagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacaaaaatgcgaaccaagtatttcaattttatgacaaaaattctcaatcgttgttacaagtgaaacgcttcgaggttacagctactattgatttaggagatcgcctatggtctcgccccggcgtcgtgcgtccgccgcgagccgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggctgctgctgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgctggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttctttaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagctgctgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtaccaagaaggccaagaacggccgcctggtgacggtatccgagggtgaagccttgattagccgctacaagatcgtaaagagcgaaaccgggcggccggagtacatcgagatcgaactggctgattggatgtaccgcgagatcacagaaggcaagaacccggacgtgctgacggttcaccccgattactttttgatcgaccccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcacggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaatgtcgac

SEQ ID NO: 60 - DNA, 12931 bp, synthetic construct
  misc_feature 1..12931: note = Polynucleotide Vector
  source 1..12931: mol_type = other DNA
taactataacggtcctaaggtagcgacgtacgtgcggagggacactatcaatcgtagctcgagtttgatatccaaaatagacgagaacaataagcaaaaactcttagttttgaaataaatcaacaatcccgagggttgtcacatatacatcaaaaacgaaaatccatatagcaaaaaaaaactctaaattaccgttcgacaaaaaagaaaactgataagacatttgctaaacattaaaaatcgattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatacctatcagccacaccaagccttccacagtcgatgaatccagaaaagcggccattttccaccatgatattcggcaagcaggcatcgccatgggtcacgacgagatcctcgccgtctggcatcctcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagcagcgcaaggaacgcccgtcgtggccagccacgatagccgcgctgcctcgtcttgaagttcattcagggcaccggacaggtcggtcttgacaaaaagaactggccttccctgcgctgacagccggaacacggcggcatcagagcagccgattgtctgttgtgcccagtcatagccgaatagcctctccacccaagcggccggagaacctgcgtgcaatccatcttgttcaatcatgtttaaactaccaccacggagacggagcacgagatggagagttgactccttctggatgttgtagtcggcaagagtacgaccatcctcaagctgctttccggcgaaaatcaaacgctgctggtctgggggaatcccttccttgtcctggatcttggctttgacattgtcgatggtgtcggaagactcaacctctagggtgatcgtcttccccgttaaggtcttcacgaagatctgcatttttgcgctagcccgaaaagcacacaatgccctgcaggcaaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctgacgcgtcatgagaagttcactcgtgacgaagggcgcgccatcacaagtttgtacaaaaaagcaggctccgaattcgcccttgcgatcgccggaccgctcgagcccgggcggccgcagccgaaccggaccgcgttgctcacgttacagctccattcctcgagccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgggaagtcatcgatacctgcaggactcttgagtactttcgccagctcacgttgacttttttcgctatcactcctttttagtgtcaaaacgtaggggtcttcgaaaatgttgagactttgcacaatctttctcaggctcgccacagttttcgtcacgatgatatttgactctggggtatagtagataatagatagaagtggtagctttactcgaaggcgaagatctgctcgatgtatttttggcgtgcagcaaatggcatccgtccagatctggtcaacacaccaagatcctagagttctactggcttctgtcccaaatacaaatggcttccgaaagataggattgtcttttagaagcgggagcatttgaagataaagaggagtattgaatttcgggcccaagggatcatagtaggcaagagtctcaggtttacctttgattgagtactgcagcaagctgggatcttctgcagtacatatttgactgtgtagcaaaccttcaagttcttccgcatcggcgtggtgtagagcattacgctgcgatggattccggcatagttaaagaaatcatggaagtaagactgctttttcttgccgttttcgtcggtaatcaccattcccggcgggatagtctgccagttcagttcgttgttcacacaaacggtgatacgtacacttttcccggcaataacatacggcgtgacatcggcttcaaatggcgtatagccgccctgatgctccatcacttcctgattattgacccacactttgccgtaatgagtgaccgcatcgaaacgcagcacgatacgctggcctgcccaacctttcggtataaagacttcgcgctgataccagacgttgcccgcataattacgaatatctgcatcggcgaactgatcgttaaaactgcctggcacagcaattgcccggctttcttgtaacgcgctttcccaccaacgctggtatacacacgtggttaaacacatctgcacaatcacaaaattaaaatgcttatttcatgtaaaataacttaaataaaaaccaatatatctgtctttttatacaataggtgttcacgaggtgaaaagattgtaaaaatatgatattattagatctttcgtcaatgttttatggtttgtcacgattcctcaaaaatgagtaataaattttgagtagtgctttagcgtttatgacctttgaaaggggaaggactgaaggaggcttaccacaaggcaggtataccagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgatgcggaagaacttgaaggtttgctacacagtcaaatatgtactgcagaagatcccagcttgctgcagtactcaatcaaaggtaaacctgagactcttgcctactatgatcccttgggcccgaaattcaatactcctctttatcttcaaatgctcccgcttctaaaagacaatcctatctttcggaagccatttgtatttgggacagaagccagtagaactctaggatcttggtgtgttgaccagatctggacggatgccatttgctgcacgccaaaaatacatcgagcagatcttcgccttcgagtaaagctaccacttctatctattatctactataccccagagtcaaatatcatcgtgacgaaaactgtggcgagcctgagaaagattgtgcaaagtctcaacattttcgaagacccctacgttttgacactaaaaaggagtgatagcgaaaaaagtcaacgtgagctggcgaaagtactcaagagtcctgcaggtctggaggatctgctagagtcagcttgtcgcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccacgcgtcacagttgaggtatttcggatcgtggcgatcgctatccccaggcgcgccaagggcgaattcgacccagctttcttgtacaaagtggtgatcggtccggttcggctgcggccgcattaccctgttatccctaatctcgtttaactatgactctcttaaggtagccaaattccggaaaacgacttaatcagctaatacaaactatcagtgtttgacaggatatattggcgggtaaacctaagagaaaagagcgtttattagaataatcggatatttaaaagggcgtgaaaaggtttatccgttcgtccatttgtatgtgcatgcggctggggcgagcaccagacggagcaccgccaggatcacgagccatatcctgacgaatgggttctcgtccaacgcccctttatcgacagtatgtgaatttgcagtgtggccgcgatcaacgcacactggccttgggtctgaagggtgatcttcaaaacgaacccccgtcttggtggtcagtgtgatgggcacatcgcttactcctcagtcaccctttgagccagataattaagaggcaaatgcaattggctcaggctgccatcgtccccccgtgcgaaacctgcacgtccgcgtcaaagaaataaccggcacctcttgctgtttttatcagttgagggcttgacggatcagcctcaagtttgcggcgcagcctcaaaatgagaacatctatactcctgtcataaacctcctcgtcgcgtactcgactggcaatgagaagttgctcgcgcgatagaacgtcgcggggtttctctaaaaacgcgaggagaagattgaactcacctgccgtaagtttcacctcaccgccagcttcggacatcaagcgacgttgcctgagattaagtgtccagtcagtaaaacaaaaagaccgtcggtctttggagcggacaacgttggggcgcacgcgcaaggcaacccgaatgcgtgctagaaactctctgatactgaacggcttagcgataaaatcacttgctcctagctcaagtgcaacaactttatccgtctcctcaaggcggtcgccactgataattatgattggaatatcagactttgccgccagattacgaacgatctcaagcccatcttcacgacctaaatctagatcaacaaccacgacatcgaccgtcgcggaagagagtactcttgtgaactgggtgctgtcggctaccgcggtcactttgaaggcgtggatcgtaaggtattcaataataagatgccgcatagcgacgtcgtcatcgacaagaagaacgtgtttcaacggctcacctttcaatctaaaatctgaacccttgttcacagcgcttgagaaattttcacgtgaaggatgtacaatcatctccagctaaatgggcagttcgtcagaattgcggctgaccgcggatgacaaaaatgcgaaccaagtatttcaattttatgacaaaaattctcaatcgttgttacaagtgaaacgcttcgaggttacagctactattgatttaggagatcgcctatggtctcgccccggcgtcgtgcgtccgccgcgagccgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggctgctgctgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgctggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttctttaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgcctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagctgctgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtaccaagaaggccaagaacggccgcctggtgacggtatccgagggtgaagccttgattagccgctacaagatcgtaaagagcgaaaccgggcggccggagtacatcgagatcgaactggctgattggatgtaccgcgagatcacagaaggcaagaacccggacgtgctgacggttcaccccgattactttttgatcgaccccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcacggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaatgtcgac

SEQ ID NO: 61 - DNA, 82 bp, Arabidopsis thaliana
  source 1..82: mol_type = unassigned DNA
tgacagaagagagtgagcacacaaaggcaatttgcatatcattgcacttgcttctcttgcgtgctcactgctctttctgtca

SEQ ID NO: 62 - protein, 180 aa, Petunia axillaris
DSLVGQALFGDGAGAIIIGSDPIPGVERPLFELVSAAQTLLPDSHGAIDGHLREVGLTFHLLKDVPGLISKNIEKSLEEAFKPLGISDWNSLFWIAHPGGPAILDQVEIKLGLKPEKLKATRNVLSDYGNMSSACVLFILDEMRKASAKEGLGTTGEGLEWGVLFGFGPGLTVETVVLHS

SEQ ID NO: 63 - DNA, 547 bp, Petunia axillaris
  source 1..547: mol_type = unassigned DNA
gcaacactgtggaggacaacagtctcaactgttagcccaggcccaaatccaaaaagaacaccccactcaagcccttcaccagtagttcctaaaccttctttggctgaggcctttctcatttcatccaaaataaacagtacacaagcacttgacatgttaccatagtcacttaacacattccttgtagccttaagtttctcgggctttaggcccaactttatttcaacttggtccaaaattgcaggcccacctggatgagcaatccagaatagagagttccaatcagaaatgcccaaaggtttaaatgcttcctcaaggctcttctcaatattttttgagatcagcccaggaacatctttgagtaagtggaatgtaagcccaacttcacggagatggccatcaatagcaccatggctatctgggagaagagtttgggctgctgaaacgagctcgaacaaaggcctctcgactcctggaattggatcagaacctataatgatcgcgcctgccccatcaccaaaaagggcttggccaactaaactatcca

SEQ ID NO: 64 - DNA, 1094 bp, Petunia axillaris
  source 1..1094: mol_type = unassigned DNA
gcaacactgtggaggacaacagtctcaactgttagcccaggcccaaatccaaaaagaacaccccactcaagcccttcaccagtagttcctaaaccttctttggctgaggcctttctcatttcatccaaaataaacagtacacaagcacttgacatgttaccatagtcacttaacacattccttgtagccttaagtttctcgggctttaggcccaactttatttcaacttggtccaaaattgcaggcccacctggatgagcaatccagaatagagagttccaatcagaaatgcccaaaggtttaaatgcttcctcaaggctcttctcaatattttttgagatcagcccaggaacatctttgagtaagtggaatgtaagcccaacttcacggagatggccatcaatagcaccatggctatctgggagaagagtttgggctgctgaaacgagctcgaacaaaggcctctcgactcctggaattggatcagaacctataatgatcgcgcctgccccatcaccaaaaagggcttggccaactaaactatccatggatagtttagttggccaagccctttttggtgatggggcaggcgcgatcattataggttctgatccaattccaggagtcgagaggcctttgttcgagctcgtttcagcagcccaaactcttctcccagatagccatggtgctattgatggccatctccgtgaagttgggcttacattccacttactcaaagatgttcctgggctgatctcaaaaaatattgagaagagccttgaggaagcatttaaacctttgggcatttctgattggaactctctattctggattgctcatccaggtgggcctgcaattttggaccaagttgaaataaagttgggcctaaagcccgagaaacttaaggctacaaggaatgtgttaagtgactatggtaacatgtcaagtgcttgtgtactgtttattttggatgaaatgagaaaggcctcagccaaagaaggtttaggaactactggtgaagggcttgagtggggtgttctttttggatttgggcctgggctaacagttgagactgttgtcctccacagtgttgc

(19) United States
(12) Patent Application Publication
(10) Pub. No.: US 2025/0290084 A1
(43) Pub. Date: Sep. 18, 2025

(54) CONTROL OF PHENOTYPE IN PLANTS

(71) Applicant: GREENVENUS, LLC, Davis, CA (US)

(72) Inventors: Sekhar BODDUPALLI, Davis, CA (US); Andrey BOUKHAROV, Melrose, MA (US); Rio STAMLER, Davis, CA (US); Zhongsen LI, Hockessin, DE (US); Arianne TREMBLAY, Davis, CA (US); Stephen SCHAUER, Rockville, MD (US); Shiv B. TIWARI, San Jose, CA (US); John SALMERON, Hillsborough, NC (US)

(21) Appl. No.: 19/220,868

(22) Filed: May 28, 2025

Related U.S. Application Data
(62) Division of application No. 18/062,394, filed on Dec. 6, 2022, now Pat. No. 12,344,852.
Application No. 18/062,394 is a division of application No. 16/316,783, filed on Jan. 10, 2019, now Pat. No. 11,555,197.
(60) Provisional application No. 62/366,402, filed on Jul. 25, 2016.
(60) Provisional application No. 62/468,012, filed on Mar. 7, 2017.
(60) Provisional application No. 62/467,958, filed on Mar. 7, 2017.

(51) Int. Cl. C12N 15/82

(52) U.S. Cl. CPC C12N 15/8217; C12N 15/8218; C12N 15/825; C12N 15/827; C12N 15/8273; C12N 15/8274; C12N 15/8281; C12N 15/8282; C12N 15/8283; C12N 15/8286

(57) ABSTRACT

The invention provides a compositions and methods for controlling phenotypic traits in plants. Genes of interest are placed under the control of a gene switch to allow inducible control or expression of a gene of interest “on-demand” by treatment of the plant with a chemical ligand.

This application is a divisional patent application of U.S. patent application Ser. No. 18/062,394, filed Dec. 6, 2022, which is a divisional patent application of U.S. patent application Ser. No. 16/316,783 filed Jan. 10, 2019, now U.S. Pat. No. 11,555,197, issued Jan. 17, 2023, which is a National Stage application under 35 U.S.C. § 371 of PCT Application No. PCT/US2017/043650, filed Jul. 25, 2017, which claims the priority benefit of U.S. Provisional Patent Application Ser. No. 62/467,958, filed Mar. 7, 2017; U.S. Provisional Patent Application Ser. No. 62/468,012, filed Mar. 7, 2017; and U.S. Provisional Patent Application Ser. No. 62/366,402, filed Jul. 25, 2016; which are hereby incorporated by reference in their entirety.

### REFERENCE TO SEQUENCE LISTING

This Sequence Listing is being submitted electronically in XML format and is hereby incorporated by reference in its entirety. Said XML copy, created on Nov. 30, 2022, is named 2574321098.xml and is 171 KB in Size. No new matter is being introduced.

### BACKGROUND OF THE INVENTION

In plants, the transition to flowering triggers global changes that can greatly influence vegetative organs in addition to initiating seed production (Andrés F and Coupland G. (2012) Nature Reviews Genetics 13, 627-639). In crops harvested for vegetative tissues, flowering can lead to losses in yield or quality as plant metabolism is redirected. For example, in sugar beets high yields depend on a prolonged vegetative growing phase, and biennial varieties are bred to avoid early flowering (Mutasa-Göttens E S et al., (2010) AoB Plants, doi: 10.1093/aobpla/plq012). In alfalfa, a delay in flowering of five days can result in forage yield increase of 450 kg per acre (Dupont Pioneer: Delayed Alfalfa Harvest (URL pioneer.com/home/site/mobile/silage-zone/alfalfa_harvest/delayed-harvest/). Alfalfa forage quality is also well-known to be inversely related to maturity, with Relative Feed Quality (RFQ) scores declining by five points for each day of delay in harvest after bud stage (Dupont Pioneer: Delayed Alfalfa Harvest (URL pioneer.com/home/site/mobile/silage-zone/alfalfa_harvest/delayed-harvest/).

A second driver for flowering control is found in crops with high rates of out-crossing. In some cases, risks of gene flow to crop relatives have added to the cost and time requirements for development of genetically engineered (GE) varieties. For example, out-crossing to the noxious weed Johnsongrass has been a major barrier to development of GE sorghum (Paterson A H and Chandler M J, Texas A&M Soil and Crop Science (at URL nbiap.vt.edu/brarg/brasym96/paterson96.htm). Deregulation of ROUNDUP READY@herbicide-tolerant turf grass by SCOTTS MIRACLE-GRO® company took more than 10 years and only succeeded when a different molecular strategy was adopted (Wang Z-Y and Brummer C E. (2010) Annals of Botany doi: 10.1093/aob/mcs).

Control of seed production also has commercial implications for crops where farmer-saved seed can erode germplasm value and pose challenges to varietal stewardship. Use of a controllable flowering system would greatly reduce such risks to the seed company.

Breeders have been successful at exploiting natural variation to tailor flowering time for increased yields (Jung C and Müller AE (2009) Trends Plant Sci. 14: 563-573). However, as reproductive transition is still under control of the plant, there is need in the art for technology to induce flowering on demand, and the ability to “toggle” back and forth between vegetative and reproductive states. Controlled delay (inhibition) or prevention (absence) of flowering can lend the positive benefits described above. Moreover, since flowering is necessary for seed production and breeding, seed companies need the ability to restore flowering competency in non-flowering cultivars when desired.

Breeders and farmers also strive to improve or alter various traits in plants including color, aroma, sweetness, nutritional value, and many other traits. Selective cross-breeding has helped achieve desired changes over generations, but the advent of genetic engineering has led to direct control over expression or alteration of various genes in plants to achieve these ends. In most cases, however, the desired traits are expressed constitutively. They cannot be controlled over life cycle of the plant and often have some negative agronomic effects, e.g. decrease in yield or fitness, directly related to their constitutive expression.

Pest and disease protection is an important aspect of plant breeding and crop production. Various strategies have been implemented to have plants express a trait that enables them to resist infection and pests. These strategies fall in several categories: expression of proteins that promote plant innate immunity (e.g. NPR1, Defensin, flagelin); expression of proteins or RNA that disrupt pest recognition modules (disruption of sensitivity genes or plant receptors by RNAi, dsRNA or peptides), and expression of RNA or peptides that kill pests (e.g. Antimicrobial peptides, defensins, RNAi/dsRNA targeting pest genes).

In other systems, double-stranded RNA mediated post-transcriptional gene silencing is a conserved eukaryotic gene regulation mechanism, whereby dsRNA molecules are processed by a Dicer-like enzyme into dsRNA molecules 20-25 nt in length, termed siRNAs. Single stranded anti-sense RNAs derived from siRNAs associate with an RNA Induced Silencing Complex (RISC) and target homologous mRNA for degradation through the catalytic activity of Argonaut-like enzymes. This conserved gene-expression control module has been extensively exploited by scientists to uncover gene function in a multitude of organisms. In addition, there is an emerging body of evidence indicating that plant-pathogenic fungi will absorb and process exogenous dsRNA molecules resulting in gene silencing of essential endogenous genes for pathogen growth and virulence. A recent publication validates Host Induced Gene Silencing (HIGS) as a potent control strategy for generating resistance to the broad host range pathogen Botrytis cinerea (Wang et al. (2016) Nature Plants 2:16151).

However, while multiple plant genes have been identified that have direct anti-microbial activity or act as positive regulators of the innate immune system, constitutive expression of these genes typically results in a loss of fitness for the plant. Similarly, constitutive expression of dsRNA against pest genes may also burden plant growth and/or fitness. Moreover, constitutive expression of such genes can accelerate (or may lead to) development of resistance to the plant pathogens and pests.

There is a need in the art to provide for controlled, “on-demand” expression of genes in plants which achieve desired ends while minimizing negative effects on the plants.

### BRIEF SUMMARY OF THE INVENTION

The invention provides vectors and polynucleotides for controlled expression of traits in plants, plants transformed with polynucleotides to allow for controlled expression of traits and methods for making and using the same.

In some embodiments, control is exerted over a native plant gene for the trait to be controlled. In other embodiments, a plant gene is replaced with a polynucleotide used to transform the plant with control elements operatively linked to the inserted trait. In other embodiments, exogenous genes are inserted that inhibit native plant traits along with controlled genes to suppress the inhibition and inducibly restore function.

The invention provides a vector or polynucleotide comprising nucleic acids encoding:
(a) an ecdysone receptor-based (EcR-based) gene switch;
(b) a nucleic acid or protein which controls a gene of interest in a plant; and,
(c) a regulatory element operably connected 5′ of b), wherein the EcR-based gene switch binds said regulatory element in the presence of a chemical ligand.

The invention provides one or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a nucleic acid which controls a trait or traits in a plant; and,
(c) a regulatory element operably connected 5′ of b), wherein the gene switch induces expression of b) in the presence of a chemical ligand.

The vectors or polynucleotides may comprise genes of interest such as a gene for control or regulation of flowering, drought resistance, herbicide resistance, pest resistance, diseases resistance, type of floral color, intensity of floral color, floral aroma, accumulation of specific nutrients, fruit aroma, taste, nutritional value, or for expression of an exogenous gene.

In some cases the expression of the exogenous gene encodes a protein that has a toxic effect on the plant or imposes a metabolic burden on the plant during expression so controlled expression is desirable.

An EcR-based gene switch may have a ligand binding domain derived from Choristoneura fumiferana (Cf). It may also contain a heterologous (non-Cf) DNA-binding domain and a heterologous (non-Cf) transcriptional transactivation domain. In some embodiments, the DNA-binding domain is a Gal4 DNA binding domain, a LexA DNA binding domain, a transcription factor DNA binding domain, a Group H nuclear receptor DNA binding domain, a steroid/thyroid hormone nuclear receptor DNA binding domain, a bacterial LacZ DNA binding domain, an EcR DNA binding domain, an ARF DNA binding domain, a bZIP DNA binding domain, a homeodomain of a plant DNA binding domain, or a synthetic DNA binding domain. In some embodiments the transcriptional transactivation domain is a Group H nuclear receptor member transactivation domain, steroid/thyroid hormone nuclear receptor transactivation domain, synthetic or chimeric transactivation domain, polyglutamine transactivation domain, basic or acidic amino acid transactivation domain, a VP16 transactivation domain, a GAL4 transactivation domain, an NF-κB transactivation domain, a BP64 transactivation domain, a B42 acidic transactivation domain (B42AD), a p53 transactivation domain, a p65 transactivation domain (p65AD), an EDLL acidic transactivation domain from AP2/ERF, a glutamine-rich transactivation domain, an ARFs or CONSTANS proteins transactivation domain, a proline-rich transactivation domain from bZIP, or an analog, combination, or modification thereof.

In certain embodiments, the DNA-binding domain is a GAL4 transcription factor DNA-binding domain and the transcriptional transactivation domain is derived from a Simian Vacuolating Virus 40 or Simian Virus 40 (SV40) or comprises a herpes virus VP16 transcriptional transactivation domain.

In particular embodiments, the CfEcR ligand binding domain comprises one or more amino acid substitutions compared to wild-type (wt) CfEcR ligand binding domain (SEQ ID NO:19), and may comprise the polypeptide sequence of SEQ ID NO:2. An EcR-based gene switch may comprise the polypeptide sequence of SEQ ID NO:1.

For activation of the EcR-based gene switch, a chemical ligand is applied which may be a non-steroidal chemical ligand, such as, but not limited to a diacylhydrazine (DAH). An example of such a diacylhydrazine is methoxyfenozide.

The regulatory element is a recognition site for a DNA binding protein portion of an EcR-based gene switch operatively linked to a promoter.

In some embodiments, the vector or polynucleotide also has a nucleic acid sequence encoding a second gene switch and a second gene of interest. The second gene of interest is operatively linked to a second regulatory element operatively linked 5′ of said second gene of interest wherein the second gene switch binds the second regulatory element in the presence of a second chemical ligand. In such embodiments, the second gene switch may be an ecdysone receptor (EcR) based gene switch, a rapamycin-based gene switch, a prokaryotic tetracycline repressor-based gene switch, a lactose repressor-operator-based gene switch, a cumate-based gene switch, or a biotin-based gene switch. The second chemical ligand may be the same or a different chemical ligand that is active for the EcR-based gene switch.

In some embodiments, the vector or polynucleotide may also have an AUX/IAA repression domain or an ERF-associated amphiphilic repression (EAR) domain.

Polynucleotides or vectors of the invention may be used to create a plant that contains these polynucleotides or vectors in which some or all of the vector or polynucleotide is integrated into the plant genome. The plant may be monocotyledonous or dicotyledonous.

The plant may be a flowering plant, a fruit or vegetable crop plant, a grain, a forage crop or a turf grass.

Examples of a fruit or vegetable crop are alfalfa sprouts, apples, apricots, artichokes, Asian pears, asparagus, atemoyas, avocados, bamboo shoots, bananas, beans, bean sprouts, beets, belgian endive, bitter melons, bell peppers, blackberries, blueberries, bok choy, boniato, boysenberries, broccoflowers, broccoli, broccolini, brussels sprouts, butter lettuce, cabbage, cantaloupe, carambola, carrots, casaba melons, cauliflower, celery, chayotes, cherimoyas, cherries, coconuts, coffee, collard greens, corn, cranberries, cucumbers, dates, eggplant, endive, escarole, feijoa, fennel, figs, garlic, gooseberries, grapefruit, grapes, green beans, green onions, collard greens, mustard greens, guava, hominy, honeydew melons, horned melons, iceberg lettuce, Jerusalem artichokes, jincama, kale, kiwifruit, kohlrabi, kumquats, leeks, lemons, lettuce, lima beans, limes, longan, loquats, lychees, madarins, malangas, marijuana, mandarin oranges, mangos, mulberries, mushrooms, napas, nectarines, okra, onions, oranges, papayas, parsnip, passion fruits, paw-paws, peaches, peanut, pears, sugar snap peas, green peas, peppers, persimmons, pineapples, plantains, plums, pomegranates, potatoes, prickly pears, pummelos, pumpkins, quince, radicchio, radishes, raspberries, red cabbage, rhubarb, romaine lettuce, rutabaga, shallots, snow peas, soybeans, spinach, sprouts, squash, strawberries, string beans, sweet potatoes, tangelo, tangerines, tomatillo, tomatoes, turnip, ugli fruit, watermelons, water chestnuts, watercress, waxed beans, yams, yellow squash, yuca/cassava, and zucchini squash.

Examples of flowering plants include, but are not limited to, African daisy, Agapanthus, Ageratum houstonianum, Alchemilla, Allium, Alyssum, Amaranthus, Amaryllis, Anemone, Angelonia, Anthurium, Artemisia, Asclepias syriaca, Aster, Astilbe, Astrantia, Aubreita deltoidea, baby's breath, bachelor button, balloon flower, bee balm, begonia, bellflower, blanketflower, Bergenia, black-eyed Susan, blanket flower, blazing star, bleeding heart, bluebell, blue-eyed grass, blue star flower, Bouvardia, Bougainvillea, broom, Buddleja, bush morning glory, buttercup, butterfly weed, butterfly bush, Calendula, California poppy, calla lily, Calliandra, Camellia, Campenula, candytuft, canna lily, cape primrose, cardinal flower, carnation, catmint, celosia, chrysanthemum, Clarkia, clover, clematis, cockscomb, columbine, coneflower, coral bells, Coreopsis, Cosmos, Cotoneaster, Crocus, creeping phlox, Crocosmia, crown imperial, cuckoo flower, Cyclamen, Dahlia, day lily, Delphinium, Echium, English bluebell, Erigeron, evening primrose, Euphorbia, flannel flower, flax flower, floss flower, forget-me-not, Forsythia, foxglove, frangipani, freesia, fuschia, gardenia, geranium, gas plant, Gaura, gayfeather, Gerbera, Gladiolus, globeflower, goldenrod, grape hyacinth, Gypsophila, heather, Hebe, Helenium, Heliotrope, Hellebore, hibiscus, hollyhock, honeysuckle, hosta, hyacinth, hydrangea, Hypericum, hardy geranium, hybrid tea roses, Iceland poppy, ice plant, Ilex, Impatiens, Ipheion uniflorum, iris, Ixia, Ixora, Jaborosa, Jacob's ladder, Jamesia americana, jasmine, Jupiter's beard, kaffir lily, Kalmia, kangaroo paw, Kerria, Knautia macedonica, Kniphofia, Kolkwitzia, lady's slipper, Lamium, Lantana, larkspur, Lavatera, lavender, Lechenaultia, lilac, lily, lily of the valley, Linaria, lisianthus, lobelia, loosestrife, lotus, lunaria, lupin, magnolia, Maltese cross, Mandevilla, Marguerite daisy, marigold, Matthiola, mayflower, Meconopsis, mimosa, Mina lobate, mock orange, monk's hood, moonflower, morning glory, Muscari, narcissus, nasturtiums, Nemesia, Nemophila, Nerine, New Guinea impatien, Nicotiana, Nierembergia, Nigella, Nolana, oleander, orchid, oriental lily, oriental poppy, Osteospermum, oyster plant, ox eye daisy, painted daisy, pansy, passion flower, peace lily, Pelargonium, Penstemon, peony, Persian buttercup, Peruvian lily, petunia, pincushion flower, pink lady's slipper, pointsettia, Polyanthus, poppy anemone, Portulaca grandiflora, Primula, Quaker ladies, Queen Anne's lace, Queen's cup, Queen of the meadow, quince, rain lily, Ranunculus, Rhododendron, rock rose, Rondeletia, rose, rose of Sharon, Salvia splendens, Saponaria, Scabiosa, Scaevola, scented geranium, Scilla, Sedum, shasta daisy, shrub roses, Silene, silver lace vine, snapdragon, snowball bush, snowdrop, snowflake, statice, strawflower, sun drop, sunflower, sweet pea, Syringa, tea rose, tiger flower, tiger lily, Tithonia, Trillium, Triteleia, Tritonia crocata, trumpet vine, tuberose, tulip, urn plant, Ursinia, Uva ursi, Verbena, Veronica incana, Vinca, Viola tri-colour, Violet, Virginia creeper, wallflower, wandflower, water lily, Watsonia, wax plant, Wedelia, Weigela, wild rose, wild violet, winter aconite, winterberry, winter jasmine, wishbone flower, wisteria, wooly violet, Xerophyllum, Xylobium, Xylosma, yarrow, yellow angel, yellow bell, yellow-eyed grass, yellowhorn, Zenobia, and zinnia.

Examples of grains are barley, buckwheat, bulgur wheat, corn, durum wheat, einkorn, emmer, farro, fonio, kamut, millet, oats, rice, rye, semolina wheat, sorghum, spelt, teff, triticale, wheat, bamboo shoots, barleygrass, lemongrass, molasses, rapadura, sugarcane, wheatgrass, Amaranth, Coxcomb, pitseed goosefoot, quinoa, chia, acacia seed, and wattleseed.

Examples of turf grasses are Kentucky bluegrass, perennial ryegrass, tall fescue, fine fescue, creeping bentgrass, creeping red fescue, hard fescue, chewings fescue, Bermudagrass, buffalograss, kikuyugrass, St. Augustine, and zoysia.

The invention also provides a vector or polynucleotide comprising nucleic acids encoding:
(a) an ecdysone receptor-based (EcR-based) gene switch;
(b) a nucleic acid or protein which controls flowering in plants; and,
(c) a regulatory element 5′ of b), wherein the EcR-based gene switch binds the regulatory element in the presence of a chemical ligand.

The invention also provides one or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a nucleic acid which controls flowering in plants; and,
(c) a regulatory element 5′ of b), wherein said gene switch induces expression of b) in the presence of a chemical ligand.

In some embodiments, the nucleic acid or protein that controls flowering is Gigantea (GI), Nuclear Factor Y (NFY), Constans (CO); protein farnesyltransferase (PFT), Agamous-Like 15 (AGL15), Flowering Locus (FT); Twin Sister of FT (TSF), bZIP transcription factor (FD), Flowering Locus T, Apetala1 (AP1), Suppressor of Overexpression of CO1 (SOC1), Agamous-Like 24 (AGL24); Leafy (LFY), or Squamosa Promoter Binding Protein-Like 3a (SPL3). In particular embodiments, the nucleic acid or protein that controls flowering is the Flowering T Locus (FT).

An EcR-based gene switch may have a ligand binding domain derived from Choristoneura fumiferana (Cf). It may also contain a heterologous (non-Cf) DNA-binding domain and a heterologous (non-Cf) transcriptional transactivation domain. In some embodiments, the DNA-binding domain is a Gal4 binding DNA binding domain, a LexA DNA binding domain, a transcription factor DNA binding domain, a Group H nuclear receptor DNA binding domain, a steroid/thyroid hormone nuclear receptor DNA binding domain, a bacterial LacZ DNA binding domain, an EcR DNA binding domain, an ARF DNA binding domain, a bZIP DNA binding domain, a homeodomain of a plant DNA binding domain, or a synthetic DNA binding domain. In some embodiments the transcriptional transactivation domain is a Group H nuclear receptor member transactivation domain, steroid/thyroid hormone nuclear receptor transactivation domain, synthetic or chimeric transactivation domain, polyglutamine transactivation domain, basic or acidic amino acid transactivation domain, a VP16 transactivation domain, a GAL4 transactivation domain, an NF-κB transactivation domain, a BP64 transactivation domain, a B42 acidic transactivation domain (B42AD), p53 transactivation domain, a p65 transtransactivation domain (p65AD), an EDLL acidic transactivation domain from AP2/ERF, a glutamine-rich transactivation domain, an ARFs or CONSTANS proteins transactivation domain, a proline-rich transactivation domain from bZIP or an analog, combination, or modification thereof.

In certain embodiments, the DNA-binding domain is a GAL4 transcription factor DNA-binding domain and the transcriptional transactivation domain is derived from a Simian Vacuolating Virus 40 (SV40) or comprises a herpes virus VP16 transcriptional transactivation domain.

In particular embodiments, the CfEcR ligand binding domain comprises one or more amino acid substitutions compared to wild-type (wt) CfEcR ligand binding domain (SEQ ID NO:19), and may comprise the polypeptide sequence of SEQ ID NO:2. The EcR-based gene switch may comprise the polypeptide sequence of SEQ ID NO:1.

For activation of the EcR-based gene switch, a chemical ligand is applied which may be a non-steroidal chemical ligand, such as, but not limited to a diacylhydrazine. An example of such a diacylhydrazine is methoxyfenozide.

The regulatory element is a recognition site for a DNA binding protein portion of the EcR-based gene switch operatively linked to a promoter. In certain embodiments, the promoter is from a plant gene responsible for flowering. Examples of promoters that may be used include, but are not limited to, promoters for the genes APETALA1 (API), LEAFY (LFY), Arabidopsis thaliana FLOWERING PROMOTING FACTOR 1 (AtFPF1), UNUSUAL FLORAL ORGANS (UFO), TERMINAL FLOWER 1 (TFL1), SHOOT MERISTEMLESS (STM), upstream region of the HISTONE H4 (H4A748), Arabidopsis thaliana TRANSLATIONALLY CONTROLLED TUMOR PROTEIN (AtTCTP), or chlorophyll a/b binding protein.

In some embodiments, the vector or polynucleotide also has a nucleic acid sequence encoding a second gene switch and a second gene of interest. The second gene of interest is operatively linked to a second regulatory element operatively linked 5′ of said second gene of interest wherein the second gene switch binds the second regulatory element in the presence of a second chemical ligand. In such embodiments, the second gene switch may be an ecdysone receptor (EcR) based gene switch, a rapamycin-based gene switch, a prokaryotic tetracycline repressor-based gene switch, a lactose repressor-operator-based gene switch, a cumate-based gene switch, or a biotin-based gene switch. The second chemical ligand may be the same or a different chemical ligand that is active for the EcR-based gene switch.

In some embodiments, the vector or polynucleotide may also have an AUX/IAA repression domain or an ERF-associated amphiphilic repression (EAR) domain.

The polynucleotides or vectors may be used to create a plant that contains these polynucleotides or vectors in which some or all of the vector or polynucleotide is integrated into the plant genome. The plant may be monocotyledonous or dictyledonous.

The invention also provides a vector or polynucleotide comprising nucleic acids encoding:
(a) an ecdysone receptor-based (EcR-based) gene switch;
(b) a protein or an RNA that inhibits a plant pathogen; and,
(c) a regulatory element, wherein said EcR-based gene switch binds said regulatory element in the presence of a chemical ligand.

The invention also provides one or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a protein or an RNA that inhibits a plant pathogen; and,
(c) a regulatory element, wherein said gene switch binds induces expression of b) in the presence of a chemical ligand.

The EcR-based gene switch may have a ligand binding domain derived from the Choristoneura fumiferana (Cf) EcR. It may also contain a heterologous (non-Cf) DNA-binding domain and a heterologous (non-Cf) transcriptional transactivation domain. In some embodiments, the DNA-binding domain is a Gal4 DNA binding domain, a LexA DNA binding domain, a transcription factor DNA binding domain, a Group H nuclear receptor DNA binding domain, a steroid/thyroid hormone nuclear receptor DNA binding domain, a bacterial LacZ DNA binding domain, an EcR DNA binding domain, an ARF DNA binding domain, a bZIP DNA binding domain, a homeodomain of a plant DNA binding domain, or a synthetic DNA binding domain. In some embodiments the transcriptional transactivation domain is a Group H nuclear receptor member transactivation domain, steroid/thyroid hormone nuclear receptor transactivation domain, synthetic or chimeric transactivation domain, polyglutamine transactivation domain, basic or acidic amino acid transactivation domain, a VP16 transactivation domain, a GAL4 transactivation domain, an NF-κB transactivation domain, a BP64 transactivation domain, a B42 acidic transactivation domain (B42AD), a p53 transactivation domain, a p65 transactivation domain (p65AD), an EDLL acidic transactivation domain from AP2/ERF, a glutamine-rich transactivation domain ARFs or CONSTANS proteins transactivation domain, a proline-rich transactivation domain from bZIP or an analog, combination, or modification thereof.

In certain embodiments, the DNA-binding domain is a GAL4 transcription factor DNA-binding domain and the transcriptional transactivation domain is derived from a Simian Vacuolating Virus 40 (SV40) or comprises a herpes virus VP16 transcriptional transactivation domain.

In particular embodiments, the CfEcR ligand binding domain comprises one or more amino acid substitutions compared to wild-type (wt) CfEcR ligand binding domain (SEQ ID NO:19), and may comprise the polypeptide sequence of SEQ ID NO:2. The EcR-based gene switch may comprise the polypeptide sequence of SEQ ID NO:1.

For activation of the EcR-based gene switch, a chemical ligand is applied which may be a non-steroidal chemical ligand, such as, but not limited to a diacylhydrazine. An example of such a diacylhydrazine is methoxyfenozide.

The regulatory element is a recognition site for a DNA binding protein portion of the EcR-based gene switch operatively linked to a promoter.

In some embodiments, the vector or polynucleotide also has a nucleic acid sequence encoding a second gene switch and a second gene of interest. The second gene of interest is operatively linked to a second regulatory element operatively linked 5′ of said second gene of interest wherein the second gene switch binds the second regulatory element in the presence of a second chemical ligand. In such embodiments, the second gene switch may be an ecdysone receptor (EcR) based gene switch, a rapamycin-based gene switch, a prokaryotic tetracycline repressor-based gene switch, a lactose repressor-operator-based gene switch, a cumate-based gene switch, or a biotin-based gene switch. The second chemical ligand may be the same or a different chemical ligand that is active for the EcR-based gene switch.

In some embodiments, the vector or polynucleotide may also have an AUX/IAA repression domain or an ERF-associated amphiphilic repression (EAR) domain.

In some embodiments, the protein or RNA that inhibits a plant pathogen is a defensin family protein, including, but not limited to, a Defensin, a Snakin, a Hevein, a Thionin, a Lipid Transfer Protein, a Cyclotides, a Shepherins, an MBP-1, Vicilin-like peptide, an Impatiens family peptide, a Beta-Barrelin or a Knottin. Examples of proteins in the defensin family include Ah-AMP1, AX1, AX2, At-AFP1 (LCR67), AFP2B, Psdl, Psd2, J1-1, J1-2, Tk-AMP-D1, Tk-AMP-D1.1, Tk-AMP-D2, Tk-AMP-D3, Tk-AMP-D4, Tk-AMP-D5, Tk-AMP-D6, Tk-AMP-D6.1, Tm-AMP-D1.2, PhD1, PhD2, NaDI, MsDefl, MtDef2, MtDef4, MtDef5, Rs-AFP1, Rs-AFP2, Sa-AFP2, VrD1, Snakin-1 (StSNI), Snakin-2 (StSN2), AC-AMP1, AC-AMP2, Ar-AMP, EAFP1, EAFP2, Ee-CBP, Fa-AMP1, Fa-AMP2, IWF4, PN-AMP1, PN-AMP2, WjAMP1, Alpha-i-Purothionin, Alpha-2-Purothionin, Alpha-hordothionin, Beta-hordothjionin, BTH6, Pp-AMP1, Pp-AMP2, Tu-AMP-1, Tu-AMP2, VtA3, VtB, La-LTP (LJAFP), Ace-AMP1, Hy-LTP Cw-18 (PKG2316), Hy-LTP4.1 (LTP4.1)(CW21), IWF1 (Bv-LTP1), IWF2 (Bv-LTP2), Pa-LTP1, Circulin-A, Circulin-B, Cyclopsychotride-A, Kalata-B1, MiAMP2, MiAMP2b, MiAMP2c-1, MiAMP2c-2, MiAMP2c-3, MiAMP2d, lb-AMP1, Ib-AMP2, Ib-AMP3, Ib-AMP4, MiAMPi MJ-AMP1, MJ-AMP2, Mc-AMP1, and Pa-AMP1 (PAFP-S).

In some embodiments, the plant pathogen is a virus, a fungus, a bacterium, or an insect.

Examples of plant viruses include, but are not limited to, Tobacco mosaic virus, Tomato spotted wilt virus, Tomato yellow leaf curl virus, Cucumber mosaic virus, Potato virus Y, Cauliflower mosaic virus, African cassava mosaic virus, Plum pox virus, Brome mosaic virus, Potato virus X, Citrus tristeza virus, Barley yellow dwarf virus, Potato leafroll virus and Tomato bushy stunt virus.

Examples of fungal pathogens include, but are not limited to, Alternaria, Botrytis, Fusarium, Pyricularia, Verticillium, Aspergillus, Saccharomyces, Trichophyton, Cercospora, Cladosporium, Leptosphaeria, Penicillium, Trichoderma, Septoria; Plectosphaerella, Colletotrichum, Bipolaris, Ascochyta, Phytophthora, Gibberella, Mycosphaerella, Neurospora, Phoma, Pythium, Rhizoctonia, Helinthosporium, Geotrichum, Sclerotinia, Clavibacter, Pyrenopkora, Nectria; Candida; Cryptococcus, Ceratocystis, Chalara, and Venturia. In certain preferred embodiments, the pathogen is Botrytis cinerea.

Examples of bacterial pathogens include, but are not limited to, Bacillus, Enterococcus, Staphylococcus, Clavibacter, Ralstonia, Listeria, Rhizobium, Erwinia, Escherichia, Pseudomonas, Agrobacterium, Sarcina, Ralstonia, Micrococcus, Proteus, Klebsiella, Salmonella, Serratia; Streptococcus, and Xanthomonas. In certain preferred embodiments, the bacterial pathogen is Erwinia amylovora.

Examples of insect pathogens include, but are not limited to, Spodoptera and Helicoverpa. In certain preferred embodiments, the insect pathogen is Spodoptera frugiperda.

In some embodiments, the RNA has sufficient self-homology to form a double-stranded RNA (dsRNA). Such dsRNAs inhibit a gene of the virus, fungus, bacterium or insect such that the plant is resistant to the detrimental effects of the pathogen.

The invention also provides a vector or polynucleotide comprising nucleic acids encoding:
(a) a gene switch;
(b) a Bacillus amyloliquefaciens Barstar protein;
(c) a Bacillus amyloliquefaciens barnase ribonuclease; and
(d) a regulatory element 5′ of (b), wherein said gene switch binds said regulatory element in the presence of a chemical ligand.

The invention also provides one or more vectors or polynucleotides comprising nucleic acids encoding:
(a) a gene switch;
(b) a Bacillus amyloliquefaciens Barstar protein;
(c) a Bacillus amyloliquefaciens barnase ribonuclease; and
(d) a regulatory element 5′ of (b), wherein said gene switch induces expression of b) in the presence of a chemical ligand.

The invention also provides one or more vectors or polynucleotides comprising nucleic acids encoding:
(a) an EcR-based gene switch or an EcR-based gene switch comprising a VGEvy ligand binding domain;
(b) a Bacillus amyloliquefaciens Barstar protein;
(c) a Bacillus amyloliquefaciens barnase ribonuclease; and
(d) a regulatory element 5′ of (b), wherein said gene switch induces expression of b) in the presence of a chemical ligand.

In some embodiments, barnase is operatively linked to a plant promoter of a plant gene of interest to suppress expression of said plant gene of interest, such as, but not limited to a gene for flowering, drought resistance, herbicide resistance, pest resistance, diseases resistance, type of floral color, intensity of floral color, floral aroma, accumulation of specific nutrients, nutritional value, or for expression of an exogenous gene. In certain embodiments, the promoters provide tissue-specific expression such as in flower-specific expression. Examples of such plant promoters for tissue-specific expression in flower tissue include, but are not limited to, the promoters of plant genes such as, but not limited to APETALA1 (API), LEAFY (LFY), Arabidopsis thaliana FLOWERING PROMOTING FACTOR 1 (AtFPF1), UNUSUAL FLORAL ORGANS (UFO), TERMINAL FLOWER 1 (TFL1), SHOOT MERISTEMLESS (STM), upstream region of the HISTONE H4 (H4A748), Arabidopsis thaliana TRANSLATIONALLY CONTROLLED TUMOR PROTEIN (AtTCTP), and chlorophyll a/b binding protein.

In these embodiments of the invention, the gene switch may be an ecdysone receptor (EcR) based gene switch, a rapamycin-based gene switch, a prokaryotic tetracycline repressor-based gene switch, a lactose repressor-operator-based gene switch, a cumate-based gene switch, or a biotin-based gene switch. In certain embodiments, the gene switch is an ecdysone receptor (EcR)-based gene switch.

The gene switch may have a ligand binding domain derived from Choristoneura fumiferana (Cf) EcR. It may also contain a heterologous (non-Cf) DNA-binding domain and a heterologous (non-Cf) transcriptional transactivation domain. In some embodiments, the DNA-binding domain is a Gal4 binding DNA domain, a LexA DNA binding domain, a transcription factor DNA binding domain, a Group H nuclear receptor DNA binding domain, a steroid/thyroid hormone nuclear receptor DNA binding domain, a bacterial LacZ DNA binding domain, an EcR DNA binding domain, an ARF DNA binding domain, a bZIP DNA binding domain, a homeodomain of a plant DNA binding domain, or a synthetic DNA binding domain. In some embodiments the transcriptional transactivation domain is a Group H nuclear receptor member transactivation domain, steroid/thyroid hormone nuclear receptor transactivation domain, synthetic or chimeric transactivation domain, polyglutamine transactivation domain, basic or acidic amino acid transactivation domain, a VP16 transactivation domain, a GAL4 transactivation domain, an NF-κB transactivation domain, a BP64 transactivation domain, a B42 acidic transactivation domain (B42AD), a p65 transactivation domain (p65AD), an EDLL acidic transactivation domain from AP2/ERF, a glutamine-rich transactivation domain, an ARFs or CONSTANS proteins transactivation domain, a proline-rich transactivation domain from bZIP or an analog, combination, or modification thereof.

In certain embodiments, the DNA-binding domain is a GAL4 transcription factor DNA-binding domain and the transcriptional transactivation domain is derived from a Simian Vacuolating Virus 40 (SV40) or comprises a herpes virus VP16 transcriptional transactivation domain.

In particular embodiments, the gene switch is an EcR-based gene switch having a ligand binding domain derived from Choristoneurafumiferana (Cf). CfEcR ligand binding domain may comprise one or more amino acid substitutions compared to wild-type (wt) CfEcR ligand binding domain (SEQ ID NO:19), and may comprise the polypeptide sequence of SEQ ID NO:2. The EcR-based gene switch may comprise the polypeptide sequence of SEQ ID NO:1.

For activation of the EcR-based gene switch, a chemical ligand is applied. Such chemical ligands may be, for example, a non-steroidal chemical ligand, such as, but not limited to a diacylhydrazine. An example of such a diacylhydrazine is methoxyfenozide.

The regulatory element may be a recognition site for a DNA binding protein portion of the EcR-based gene switch operatively linked to a promoter.

In some embodiments, the vector or polynucleotide also has a nucleic acid sequence encoding a second gene switch and a second gene of interest. The second gene of interest is operatively linked to a second regulatory element operatively linked 5′ of said second gene of interest wherein the second gene switch binds the second regulatory element in the presence of a second chemical ligand. In such embodiments, the second gene switch may be an ecdysone receptor (EcR) based gene switch, a rapamycin-based gene switch, a prokaryotic tetracycline repressor-based gene switch, a lactose repressor-operator-based gene switch, a cumate-based gene switch, or a biotin-based gene switch. The second chemical ligand may be the same or a different chemical ligand that is active for the EcR-based gene switch.

In some embodiments, the vector or polynucleotide may also have an AUX/IAA repression domain or an ERF-associated amphiphilic repression (EAR) domain.

The polynucleotides or vectors may be used to create a plant that contains these polynucleotides or vectors in which some or all of the vector or polynucleotide is integrated into the plant genome. The plant may be monocotyledonous or dictyledonous.

The plant may be a flowering plant, a fruit or vegetable crop plant, a grain, or a turf grass.

Examples of a fruit or vegetable crop are alfalfa sprouts, apples, apricots, artichokes, Asian pears, asparagus, atemoyas, avocados, bamboo shoots, bananas, beans, bean sprouts, beets, belgian endive, bitter melons, bell peppers, blackberries, blueberries, bok choy, boniato, boysenberries, broccoflowers, broccoli, broccolini, brussels sprouts, butter lettuce, cabbage, cantaloupe, carambola, carrots, casaba melons, cauliflower, celery, chayotes, cherimoyas, cherries, coconuts, coffee, collard greens, corn, cranberries, cucumbers, dates, eggplant, endive, escarole, feijoa, fennel, figs, garlic, gooseberries, grapefruit, grapes, green beans, green onions, collard greens, mustard greens, guava, hominy, honeydew melons, horned melons, iceberg lettuce, Jerusalem artichokes, jincama, kale, kiwifruit, kohlrabi, kumquats, leeks, lemons, lettuce, lima beans, limes, longan, loquats, lychees, madarins, malangas, marijuana, mandarin oranges, mangos, mulberries, mushrooms, napas, nectarines, okra, onions, oranges, papayas, parsnip, passion fruits, paw-paws, peaches, peanut, pears, sugar snap peas, green peas, peppers, persimmons, pineapples, plantains, plums, pomegranates, potatoes, prickly pears, pummelos, pumpkins, quince, radicchio, radishes, raspberries, red cabbage, rhubarb, romaine lettuce, rutabaga, shallots, snow peas, soybeans, spinach, sprouts, squash, strawberries, string beans, sweet potatoes, tangelo, tangerines, tomatillo, tomatoes, turnip, ugli fruit, watermelons, water chestnuts, watercress, waxed beans, yams, yellow squash, yuca/cassava, and zucchini squash.

Examples of flowering plant include, but are not limited to, African daisy, Agapanthus, Ageratum houstonianum, Alchemilla, Allium, Alyssum, Amaranthus, Amaryllis, Anemone, Angelonia, Anthurium, Artemisia, Asclepias syriaca, Aster, Astilbe, Astrantia, Aubreita deltoidea, baby's breath, bachelor button, balloon flower, bee balm, begonia, bellflower, blanketflower, Bergenia, black-eyed Susan, blanket flower, blazing star, bleeding heart, bluebell, blue-eyed grass, blue star flower, Bouvardia, Bougainvillea, broom, Buddleja, bush morning glory, buttercup, butterfly weed, butterfly bush, Calendula, California poppy, calla lily, Calliandra, Camellia, Campenula, candytuft, canna lily, cape primrose, cardinal flower, carnation, catmint, celosia, chrysanthemum, Clarkia, clover, clematis, cockscomb, columbine, coneflower, coral bells, Coreopsis, Cosmos, Cotoneaster, Crocus, creeping phlox, Crocosmia, crown imperial, cuckoo flower, Cyclamen, Dahlia, day lily, Delphinium, Echium, English bluebell, Erigeron, evening primrose, Euphorbia, flannel flower, flax flower, floss flower, forget-me-not, Forsythia, foxglove, frangipani, freesia, fuschia, gardenia, geranium, gas plant, Gaura, gayfeather, Gerbera, Gladiolus, globeflower, goldenrod, grape hyacinth, Gypsophila, heather, Hebe, Helenium, Heliotrope, Hellebore, hibiscus, hollyhock, honeysuckle, hosta, hyacinth, hydrangea, Hypericum, hardy geranium, hybrid tea roses, Iceland poppy, ice plant, Ilex, Impatiens, Ipheion uniflorum, iris, Ixia, Ixora, Jaborosa, Jacob's ladder, Jamesia americana, jasmine, Jupiter's beard, kaffir lily, Kalmia, kangaroo paw, Kerria, Knautia macedonica, Kniphofia, Kolkwitzia, lady's slipper, Lamium, Lantana, larkspur, Lavatera, lavender, Lechenaultia, lilac, lily, lily of the valley, Linaria, lisianthus, lobelia, loosestrife, lotus, lunaria, lupin, magnolia, Maltese cross, Mandevilla, Marguerite daisy, marigold, Matthiola, mayflower, Meconopsis, mimosa, Mina lobate, mock orange, monk's hood, moonflower, morning glory, Muscari, narcissus, nasturtiums, Nemesia, Nemophila, Nerine, New Guinea impatien, Nicotiana, Nierembergia, Nigella, Nolana, oleander, orchid, oriental lily, oriental poppy, Osteospermum, oyster plant, ox eye daisy, painted daisy, pansy, passion flower, peace lily, Pelargonium, Penstemon, peony, Persian buttercup, Peruvian lily, petunia, pincushion flower, pink lady's slipper, pointsettia, Polyanthus, poppy anemone, Portulaca grandiflora, Primula, Quaker ladies, Queen Anne's lace, Queen's cup, Queen of the meadow, quince, rain lily, Ranunculus, Rhododendron, rock rose, Rondeletia, rose, rose of Sharon, Salvia splendens, Saponaria, Scabiosa, Scaevola, scented geranium, Scilla, Sedum, shasta daisy, shrub roses, Silene, silver lace vine, snapdragon, snowball bush, snowdrop, snowflake, statice, strawflower, sun drop, sunflower, sweet pea, Syringa, tea rose, tiger flower, tiger lily, Tithonia, Trillium, Triteleia, Tritonia crocata, trumpet vine, tuberose, tulip, urn plant, Ursinia, Uva ursi, Verbena, Veronica incana, Vinca, Viola tri-colour, Violet, Virginia creeper, wallflower, wandflower, water lily, Watsonia, wax plant, Wedelia, Weigela, wild rose, wild violet, winter aconite, winterberry, winter jasmine, wishbone flower, wisteria, wooly violet, Xerophyllum, Xylobium, Xylosma, yarrow, yellow angel, yellow bell, yellow-eyed grass, yellowhorn, Zenobia, and zinnia.

Examples of grain are barley, buckwheat, bulgur wheat, corn, durum wheat, einkorn, emmer, farro, fonio, kamut, millet, oats, rice, rye, semolina wheat, sorghum, spelt, teff, triticale, wheat, bamboo shoots, barleygrass, lemongrass, molasses, rapadura, sugarcane, wheatgrass, Amaranth, Coxcomb, pitseed goosefoot, quinoa, chia, acacia seed, and wattleseed.

Examples of turf grass are Kentucky bluegrass, perennial ryegrass, tall fescue, fine fescue, creeping bentgrass, creeping red fescue, hard fescue, chewings fescue, Bermudagrass, buffalograss, kikuyugrass, St. Augustine, and zoysia.

The invention also provides a vector or polynucleotide comprising nucleic acids encoding:
(a) an ecdysone receptor-based (EcR-based) gene switch;
(b) an immutans variegation mutant (IM); and,
(c) a regulatory element 5′ of b), wherein said EcR-based gene switch binds said regulatory element in the presence of a chemical ligand.

The invention also provides one or more vectors or polynucleotides comprising:
a) a polynucleotide encoding an ecdysone receptor-based (EcR-based) gene switch or an ecdysone receptor-based (EcR-based) gene switch comprising a VGEvy ligand binding domain;
b) an immutans variegation mutant (IM) nucleic acid; and,
c) a regulatory element 5′ of b), wherein said gene switch induces expression of b) in the presence of a chemical ligand

The EcR-based gene switch may have a ligand binding domain derived from Choristoneura fumiferana (Cf) EcR. It may also contain a heterologous (non-Cf) DNA-binding domain and a heterologous (non-Cf) transcriptional transactivation domain. In some embodiments, the DNA-binding domain is a Gal4 DNA binding domain, a LexA DNA binding domain, a transcription factor DNA binding domain, a Group H nuclear receptor DNA binding domain, a steroid/thyroid hormone nuclear receptor DNA binding domain, a bacterial LacZ DNA binding domain, an EcR DNA binding domain, an ARF DNA binding domain, a bZIP DNA binding domain, a homeodomain of a plant DNA binding domain, or a synthetic DNA binding domain. In some embodiments the transcriptional transactivation domain is a Group H nuclear receptor member transactivation domain, steroid/thyroid hormone nuclear receptor transactivation domain, synthetic or chimeric transactivation domain, polyglutamine transactivation domain, basic or acidic amino acid transactivation domain, a VP16 transactivation domain, a GAL4 transactivation domain, an NF-κB transactivation domain, a BP64 transactivation domain, a B42 acidic transactivation domain (B42AD), a p53 transactivation domain, a p65 transactivation domain (p65AD), an EDLL acidic transactivation domain from AP2/ERF, a glutamine-rich transactivation domain, an ARFs or CONSTANS proteins transactivation domain, a proline-rich transactivation domain from bZIP or an analog, combination, or modification thereof.

In certain embodiments, the DNA-binding domain is a GAL4 transcription factor DNA-binding domain and the transcriptional transactivation domain is derived from a Simian Vacuolating Virus 40 (SV40) or comprises a herpes virus VP16 transcriptional transactivation domain.

In particular embodiments, the CfEcR ligand binding domain comprises one or more amino acid substitutions compared to wild-type (wt) CfEcR ligand binding domain (SEQ ID NO:19), and may comprise the polypeptide sequence of SEQ ID NO:2. The EcR-based gene switch may comprise the polypeptide sequence of SEQ ID NO:1.

For activation of the EcR-based gene switch, a chemical ligand is applied which may be a non-steroidal chemical ligand, such as, but not limited to a diacylhydrazine. An example of such a diacylhydrazine is methoxyfenozide.

The regulatory element is a recognition site for a DNA binding protein portion of the EcR-based gene switch operatively linked to a promoter.

In some embodiments, the vector or polynucleotide also has a nucleic acid sequence encoding a second gene switch and a second gene of interest. The second gene of interest is operatively linked to a second regulatory element operatively linked 5′ of said second gene of interest wherein the second gene switch binds the second regulatory element in the presence of a second chemical ligand. In such embodiments, the second gene switch may be an ecdysone receptor (EcR) based gene switch, a rapamycin-based gene switch, a prokaryotic tetracycline repressor-based gene switch, a lactose repressor-operator-based gene switch, a cumate-based gene switch, or a biotin-based gene switch. The second chemical ligand may be the same or a different chemical ligand that is active for the EcR-based gene switch.

In some embodiments, the vector or polynucleotide may also have an AUX/IAA repression domain or an ERF-associated amphiphilic repression (EAR) domain.

The polynucleotides or vectors may be used to create a plant that contains these polynucleotides or vectors in which some or all of the vector or polynucleotide is integrated into the plant genome. The plant may be monocotyledonous or dictyledonous.

The invention provides a vector or polynucleotide comprising nucleic acids encoding:
(a) an ecdysone receptor-based (EcR-based) gene switch;
(b) a nucleic acid or protein which controls a gene of interest in a plant; and,
(c) a regulatory element operably connected 5′ of b), wherein said EcR-based gene switch binds said regulatory element in the presence of a chemical ligand.

The invention provides one or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a nucleic acid which controls a trait or traits in a plant; and,
(d) a regulatory element operably connected 5′ of b), wherein said gene switch induces expression of b) in the presence of a chemical ligand.

The invention also provides a method of reducing environmental risk of pollen drift from genetically engineered plants to wild plant species wherein polynucleotides or vectors of the invention are introduced to genetically engineered plants to control, inhibit and prevent cross-pollination of wild plant species. In some embodiments, the genetically engineered plants are forage crops or turfgrass.

The invention also provides a method of improving crop yield comprising introduction of polynucleotides or vectors of the invention into crop plants. In some embodiments, the crop plants are fruits or vegetables. The fruits and vegetables may be any of those described above. In some particular embodiments, the fruits are oranges or any other citrus fruit, pears, cherries, avocados, strawberries, pineapples, or apples.

The invention also provides a method of generating plants with “on-demand” resistance to pests or biological stressors comprising introduction of polynucleotides or vectors of the invention into said plants. In some embodiments, the biological stressors are drought, or herbicides.

The invention also provides a method of generating precise control of flowering in high value fruits, vegetables or other plant produce to aid in harvest timing, comprising introduction of polynucleotides or vectors of the invention into said plants. In some embodiments, the fruits are oranges or any other citrus fruit, pears, cherries, avocados, strawberries, pineapples, or apples.

The invention also provides a method of controlling flowering in plants wherein commercial value is derived from the flower, comprising introduction of polynucleotides or vectors of the invention into said plants.

The invention also provides a method of controlling plant traits, such as but not limited to, color and aroma, in fruits and flowers, comprising introduction of polynucleotides or vectors of the invention into said plants.

The invention also provides a method of increasing plant-based production of high-value active pharmaceutical ingredients, comprising introduction of polynucleotides or vectors of the invention into said plants.

The invention also provides a method of generating increased efficiency or effectiveness in methods of commercial seed production, comprising introduction of polynucleotides or vectors of the invention into said plants.

The invention also provides a method of controlling when a crop plant flowers to increase biomass production, comprising introduction of polynucleotides or vectors of the invention into said plants. In some embodiments, forage crop quality may be improved, such as by reduced lignin.

The invention also provides a method of controlling timing of fruiting and seed production in plants, comprising introduction of polynucleotides or vectors of the invention into said plants.

The invention provides a method of controlling or regulating flowering in plants comprising introducing into the plant a vector or polynucleotide comprising nucleic acids encoding:
(a) an ecdysone receptor-based (EcR-based) gene switch;
(b) a nucleic acid or protein which controls a gene of interest in a plant; and,
(c) a regulatory element operably connected 5′ of (b), wherein said EcR-based gene switch binds said regulatory element in the presence of a chemical ligand and contacting the plant with a non-steroidal chemical ligand when one desires to express the gene of interest.

The invention provides a method of controlling or regulating flowering in plants comprising introducing into the plant one or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a nucleic acid which controls or regulates flowering in a plant;
(c) a regulatory element operably connected 5′ of (b), wherein said gene switch induces expression of b) in the presence of a non-steroidal chemical ligand; and
(d) contacting the plant with the non-steroidal chemical ligand to regulate flowering.

The chemical ligand may be a diacylhydrazine, such as, for example, methoxyfenozide.

### BRIEF DESCRIPTION OF THE DRAWINGS

FIG. 1 shows DNA constructs for controlled expression of green fluorescent protein reporter gene (GFP). Panel A shows ID38 which was constructed to express GFP under the control of a switch that may be activated by addition of ligand; Panel B shows ID68, a construct that serves as a control expressing GFP constitutively.

FIG. 2 shows fluorescence in detached arabidopsis leaf disks obtained from ID38 plants (top row); wild type plants (middle row) and ID68 plants (bottom row). Fluorescence was assessed after 48 hours in water (left column) or 80 nM solution of methoxyfenozide (right column) added to activate GFP expression.

FIG. 3 shows a DNA construct for controlled expression of the flowering locus T gene (FT). Panel A shows ID14 which was constructed to express FT under the control of a switch that may be activated by addition of ligand; Panel B shows ID16, a construct that constitutively expresses FT (control).

FIG. 4 shows transgenic T1 plants produced by transforming Arabidopsis late-flowering ft mutant with constructs ID14 (left) and ID16 (right). Left—EcR-based gene switch plants (ID14) exhibit late-flowering phenotype in the absence of switch activating ligand; right—control plants (ID16) overexpress FT and exhibit premature flowering.

FIG. 5 shows a DNA construct for controlled expression of the immutans gene (IM). Panel A shows ID13 which may be constructed to express IM under the control of a switch that may be activated by addition of ligand; Panel B shows ID15, a construct that constitutively expresses IM (control).

FIG. 6 shows a representative, hypothetical construct containing a CRISPR and an EcR-based gene switch for regulating expression of the flowering locus T gene (FT) with a native FT promoter (FT Pro) that is inserted into the genome of the plant by CRISPR to place the EcR-based gene switch-controlled FT in the native position of wt FT with the native FT promoter.

FIG. 7 shows a representative, hypothetical construct containing a CRISPR and an EcR-based gene switch for regulating expression of FT gene with a strong constitutive promoter (35S) that is inserted into the genome of the plant by CRISPR to place the EcR-based gene switch-controlled FT in the native position of wt FT with the strong heterologous promoter.

FIG. 8 shows a diagram showing interaction of other genes in flowering control that may be used in creating switchable mutations; GI, GIGANTEA; NFY, Nuclear Factor Y; CO, CONSTANS; PFT, protein farnesyltransferase; AGL15, AGAMOUS-LIKE 15; FT, FLOWERING LOCUS T; TSF, TWIN SISTER OF FT; FD, bZIP transcription factor; APi, APETALA1; SOC1, SUPPRESSOR OF OVEREXPRESSION of CO1; AGL24, AGAMOUS-LIKE 24; LFY, LEAFY; SPL3, SQUAMOSA PROMOTER BINDING PROTEIN-LIKE 3. The diagram illustrates a range of interacting regulators using arabidopsis genes as an example. The orthologs of these genes from other plants may be equally useful in creating switchable control of the flowering.

FIG. 9 shows a representative, hypothetical construct containing a CRISPR and EcR-based gene switch for regulating expression of IM that is inserted into the genome of the plant by CRISPR to place the EcR-based gene switch-controlled IM in the native position of wild-type (wt) IM.

FIG. 10 shows a representative, hypothetical construct containing a CRISPR and an EcR-based gene switch for regulating expression of IM with a strong promoter (35S) that is inserted into the genome of the plant by CRISPR to place the EcR-based gene switch-controlled IM in the native position of wt IM with the strong heterologous promoter.

FIG. 11 shows a vector construct to provide constant expression of the barnase ribonuclease from Bacillus amyloliquefaciens in flower primordia to suppress flowering and EcR-based gene switch for regulating expression of barstar which inhibits barnase. Addition of the ligand methoxyfenozide turns on transcription of barstar, resulting in flowering.

FIG. 12 shows how plants expressing barnase exhibit inhibition of flowering and induction of barstar restores flowering; Panel A shows wild type Arabidopsis on the left and Barnase/Barstar plants on the right before addition of ligand; Panel B shows a close up of the Barnase/Barstar plant in A (magnified in inset); Panel C shows flowering Barnase/Barstar plants treated with 2.5 μM ligand (magnified in inset).

FIG. 13 shows how plants expressing barnase exhibit inhibition of flowering and induction of barstar restores flowering. Panel A shows number of plants in 3 genotype groups—transgenic ID06 plants with Barnase/IP:barstar construct (B/FS:B), ID06 null segregants (Null) and wild type Arabidopsis (WT)—treated with methoxyfenozide ligand at different concentrations: 0 nm (control), 50 nM and 250 nM. Each bar corresponds to the total number of plants in the group and shows the number of plants that did and did not flower. Panel B shows average time after treatement started (DPT—days post-treatment) before the first flower was observed in the same groups of plants. The error bars are Standard Error of the Mean.

FIG. 14 shows induction of expression of barstar gene in floral organs and leaves of transgenic ID06 plants with barnase/IP:barstar constructs (B/FS:B), ID06 null segregants (Null) and wild type Arabidopsis plants (WT) when plants are treated with 250 nM methoxyfenozide ligand. 0 nM ligand represents water treatment control.

FIG. 15 shows the copy number of transgenes in transgenic T1 ID14 and ID16 plants determined by quantitative PCR. Transgenic plants that showed late flowering phenotype are indicated by an asterisk (*).

FIG. 16 shows baseline expression of FT gene in T1 transgenic (ID14 and ID16) and wild type Arabidopsis plants determined by quantitative RT-PCR. Transgene copy number is shown for each transgenic plants and the plants that showed late flowering phenotype are indicated by an asterisk (*).

FIG. 17 shows controlled complementation of Arabidopsis ft mutation in ID14-59 T2 plants; The three pots on the left show untreated plants with controlled flowering plants (ID14) on the left, wild type plants in middle and late-flowering ft mutant plants on the right. The pot on the far right shows the controlled flowering plants treated with 2500 nM methoxyfenozide.

FIG. 18 documents observations done on Arabidopsis plants of 3 genotypes—homozygous single-copy transgenic T3 plants (ID14-50), ID14-50 null segregants and ft mutant plants—over period of 90 days after planting. For each plant the following 2 metrics were documented on the day when its first flower bud appeared—the age of the plant and the number of rosette leaves it has—and plotted as a marker on Age vs Number of Leaves axes as shown. Shape of the markers corresponds to each plant's genotype: black filled circles represent ft mutant plants, grey plus signs—ID14-50 homozygous T3 plants, and grey minus signs—ID14-50 nulls. Panel A shows plants in the control group treated with water. Panel B shows plants treated with methoxifenozide (50-250 nM) every other day starting at day 24.

FIG. 19 shows the averages for the age and number of leaves at the start of the flowering for homozygous single-copy transgenic T3 plants (ID14-50) and ID14-50 null segregants shown in FIG. 18. The error bars are Standard Error of the Mean.

FIG. 20 shows constructs ID158 (SEQ ID NO:59) (Panel A) which constitutively expresses a chalcone synthase A (ChsA) hairpin transcript to down regulate ChsA through gene silencing and ID164 (SEQ ID NO:55) (Panel B) which expresses ChsA hairpin transcript (SEQ ID NO:64) under the control of the switch of the invention.

FIG. 21 shows petunia flowers of varying purple color in relation to the amount of ChsA transcript present in the plant cell (not to scale). At low levels of transcript (right picture) flowers are white in color; at intermediate levels of transcript (middle picture) flowers are variegated with white and purple color; at high levels of transcript (left picture) flowers are purple.

FIG. 22 shows FS:ChsA RNAi plants before treatment (top row): FS43-4 (Panel A), FS87-2 (Panel B) and FS1 (Panel C); and after treatment (bottom row): FS43-4 (Panel D), FS87-2 (Panel E) and FS1 (Panel F) with water (control treatment).

FIG. 23 shows FS:ChsA RNAi plants before treatment (top row): FS85-6 (Panel A), FS87 (Panel B), F88 (Panel C) and FS90 (Panel D); and 22 days after treatment (bottom row) FS85-6 (Panel E), FS87 (Panel F), FS88 (Panel G) and FS90 (Panel H) with 1 mM methoxyfenozide (Mtf).

FIG. 24 shows FS:ChsA RNAi plants before treatment (top row): FS114 (Panel A), FS119 (Panel B), FS134 (Panel C) and FS146-2 (Panel D); and 6 days after treatment (bottom row) FS114 (Panel E), FS119 (Panel F), FS134 (Panel G) and FS146-2 (Panel H) with 2 mM methoxyfenozide (Mtf).

FIG. 25 shows FS:ChsA RNAi plants before treatment (top row): FS1 (Panel A), FS89 (Panel B), FS138-2 (Panel C) and FS139 (Panel D); and 4 days after treatment (bottom row) FS1 (Panel E), FS89 (Panel F), FS138-2 (Panel G) and FS139 (Panel H) with 5 mM methoxyfenozide.

FIG. 26 shows expected outcome of Agro-infiltration on constitutively expressing DCL1/DCL2/GUS hairpin petunia lines. In wild-type plants, Agro-infiltration of a construct for constitutive expression of GUS and stained with X-gluc is expected to turn the leaf area blue (left), while in a DCL1/DCL2/GUS hairpin expressing plant, the Agro-infiltration a construct for constitutive expression of GUS and stained with X-gluc is expected to have no effect and no coloration change (right).

FIG. 27 shows characterization of constitutive DCL1/DCL2/GUS hairpin transformed petunia lines for GUS silencing and Botrytis cinerea disease resistance. Panel A: wild-type (WT) and transgenic DCL1/DCL2/GUS Hairpin Petunia events were infiltrated with Agrobacterium containing 35S-GUS and stained with X-gluc; the WT parental line showing full GUS expression; transgenic line DCL-6 which does not silence GUS expression (non-functional event); and line DCL-25 which shows complete GUS silencing; Panel B: inoculation of the same lines with Botrytis cinerea.

FIG. 28 shows ligand inducible GUS hairpin expression vs constitutive GUS hairpin expression when treated with ligand and Agro-infiltrated with GUS. Panel A: expected outcomes of inducible and constitutive expression; Panel B: experimental results of three petunia lines treated with ligand and Agro-infiltrated with GUS; constitutively silenced (DCL-25) and null event (DCL-6) showed no response to ligand application. Inducible hairpin line (DCL-8) shows GUS silencing (lack of blue color) only when treated with ligand.

FIG. 29 shows Switch-DCL1/DCL2/GUS hairpin ligand-inducible Botrytis cinerea resistance. Plants were treated with 50 M methoxyfenozide or control (water) and inoculated with Botrytis cinerea 3 days after treatment. Leaves were photographed seven days after pathogen inoculation; Row A shows a switch-controlled DCL dsRNA; Row B shows a constitutively expressing DCL dsRNA (a positive control); and Row C shows a constitutively expressing non-specific dsRNA (negative control for DCL).

FIG. 30 shows a composite of switch-coupled constructs with ligand (50 M methoxyfenozide) or control (water). Row A shows a constitutively-expressing DCL dsRNA (positive control); Row B shows a switch-controlled DCL dsRNA.

FIG. 31 shows constructs for increasing biomass. A: a control of flowering construct to allow constitutive expression of miR156 (SEQ ID NO:61), an miRNA that represses the expression of SPL flowering gene(s) and a gene switch with a repression domain (LxLxL) such that when ligand is present miR156 is repressed and flowering occurs; B: a control of flowering construct to allow constitutive expression of miR156, however, in the presence of ligand, the switch leads to expression of SPL′ which is a modified SPL with nucleotide changes that encode the same amino acid sequences but make the gene resistant to miR156 silencing, thereby leading to flowering in the presence of ligand; C: a control of flowering construct to allow constitutive expression of Vegl/RNAi which silences the VEG1 gene, but in the presence of ligand, the gene switch leads to expression of PIM/AP1 and restores flowering.

FIG. 32 shows a Dicer-like Glucuronidase hybrid hairpin (DCL1/DCL2/GUS Hairpin) (SEQ ID NO:35) construct that was developed for transgenic-based disease resistance in Petunias.

### DETAILED DESCRIPTION OF THE INVENTION

All journal articles or other publications, patents and patent applications referred to herein are expressly incorporated by reference as if each individual journal article, publication, patent, or patent application was specifically and individually indicated to be incorporated by reference. In the event of a conflict between a disclosure in the present application compared to a disclosure incorporated by reference, the disclosure in the present application controls.

The present invention comprises compositions and methods useful for chemically-regulated control of flowering in dicotyledonous plants.

### Definitions

In this disclosure, a number of terms and abbreviations are used. The following definitions are provided to be helpful in understanding the scope and practice of the present invention.

As used herein, “EcR-based gene switch” is a chimeric (i.e., three-part heterologous) polypeptide comprised of a transcriptional transactivator domain, a DNA-binding domain and an EcR (ecdysone receptor-derived) ligand binding domain.

As used herein, “LB” refers to the left T-DNA border of a vector.

As used herein, “35S” refers to a cauliflower mosaic virus terminator when shown as a rectangular box in the figures.

As used herein, “35S promoter” refers to the promoter from the cauliflower mosaic virus containing TATA Box elements. In the figures “35S” refers to the full 35S promoter when shown as an arrow-shaped polygon.

As used herein, “NPTII” refers to the neomycin phosphotransferase II gene product which, when expressed, confers resistance to the antibiotic Kanamycin.

As used herein, “IP” (i.e., Inducible Promoter) refers to sequences comprising DNA binding site sequences (such as repeated (5×) GAL4 binding site sequences) and a promoter region (such as a 35S minimal promoter) that is responsive to the Activator.

As used herein, “GFP” refers to Green Fluorescent Protein.

As used herein, “Activator” refers to an EcR-based gene switch polypeptide containing a transactivation domain, a DNA-binding domain and a ligand binding domain.

As used herein, “IM” refers to the immutans gene product which is encoded by the immutans gene, im.

As used herein, “RB” refers to the right T-DNA border of a vector.

As used herein, “U6” refers to a small nuclear RNA subunit involved in RNA processing. As used herein when shown describing a construct with an arrow icon, the U6 promoter is being shown.

As used herein, “gRNA” refers to guide RNA, a nucleic acid moiety that recruits and directs nuclease activity in the CRISPR-Cas9 system. The gRNA may be composed of two molecules that form a partial double-stranded molecule including a transactivating CRSPR RNA (tacrRNA) portion and a CRISPR RNA (crRNA) portion, or it may be a nucleic acid molecule single guide RNA (sgRNA) that consists of both the crRNA and tracrRNA as a single construct.

As used herein, “Cas9” refers to the Cas9 endonuclease which is the nuclease that cleaves DNA in the CRISPR-Cas9 system.

As used herein, “IM Pro” refers to the immutans promoter.

As used herein, “IM CDS” refers to the immutans coding DNA sequence.

As used herein, “PDS” refers to phytoene desaturase which, when reduced, leads to photobleaching in plants and loss of green pigment due to reduced PDS levels.

As used herein, “FT” refers to the Flowering Locus T, a floral integrator that activates flowering in Arabidopsis thaliana and in many other plants.

As used herein, “FT Pro” refers to the flowering locus T promoter.

As used herein, “5′ FT” refers to the 5′ region of the gene encoding the Flowering Locus T.

As used herein, “FT CDS” refers to the Flowering Locus T coding DNA sequence.

As used herein, wt: means “wild-type.”

As used herein, “Bar” refers to the bar gene (phosphinothricin N-acetyltransferase) which confers phosphinothricin (PPT)-resistance.

As used herein, “Barstar” refers to a small protein from Bacillus amyloliquefaciens which inhibits barnase, resulting in flowering.

As used herein, “Barnase” refers to the barnase ribonuclease from Bacillus amyloliquefaciens which, when expressed in plants, suppresses flowering.

As used herein, “NOS” refers to the nopaline synthase promoter from Agrobacterium tumefaciens

As used herein, “E9” refers to the 3′ end (untranslated region) of the small subunit of Pisum sativum ribulose-1,5-biphosphate carboxylase (rbc).

As used herein, “AP1” refers to Apetala1,” one of three gene products of plants that are involved in the activity of APETALA3 (AP3) and PISTILLATA (PI) which are needed for floral meristem formation in Arabidopsis and other plants. As used herein when shown describing a construct with an arrow icon, the APi promoter is being shown.

As used herein, “3A” refers to the 3′ end of the small subunit of Pisum sativum ribulose-1,5-biphosphate carboxylase (rbc) poly-A (untranslated) region.

As used herein, “VGEvy” refers to a modified ecdysone receptor-based gene switch, for example, but not limited to the monopartite gene switch polypeptide VGEvy (E68V/V184I/Y204E) as shown in SEQ ID NO:1 (the (E68V/V184I/Y204E) numbering is with respect to the ligand-binding domain shown in SEQ ID NO:2).

As used herein, “LFY” refers to “Leafy,” one of three gene products of plants that are involved in the activity of APETALA3 (AP3) and PISTILLATA (PI) which are needed for proper development of flowers in Arabidopsis and other plants. As used herein when shown describing a construct with an arrow icon, the LFY promoter is being shown.

As used herein, “UFO” refers to “Unusual Floral Organs,” one of three gene products of plants that are involved in the activity of APETALA3 (AP3) and PISTILLATA (PI) which are needed for proper development of flowers in Arabidopsis and other plants. As used herein when shown describing a construct with an arrow icon, the UFO promoter is being shown.

As used herein, “GUS” refers to P-glucuronidase which is used in assays to detect expression as expression of GUS allows cleavage of substrates such as 5-bromo-4-chloro-3-indolyl glucuronide and p-nitrophenyl β-D-glucuronide to provide a blue color.

As used herein, “H4A” refers to the histone 4 gene cluster. As used herein when shown describing a construct with an arrow icon, the H4A promoter is being shown.

As used herein, “ft” refers to the gene encoding the Flowering Locus T (FT) protein.

The term “about” or “approximately” means a value or number encompassing more than and/or less than the exact value or number indicated as would be understood by one of ordinary skill in the relevant art in the context of the subject matter in which the term “about” or “approximately” is used. For example, depending on the subject matter context, “about” or “approximately” could mean within 20%, within 10%, within 5%, or even within 1% of a given value, number or range.

The term “substantially free” means that a composition comprising “A” (where “A” is a single protein, DNA molecule, vector, recombinant host cell, etc.) is substantially free of “B” (where “B” comprises one or more contaminating proteins, DNA molecules, vectors, etc.) when at least about 75% by weight of the proteins, DNA, vectors (depending on the category of species to which A and B belong) in the composition is “A”. Preferably, “A” comprises at least about 90% by weight of the A+B species in the composition, most preferably at least about 99% by weight. It is also preferred that a composition, which is substantially free of contamination, contain only a single molecular weight species having the activity or characteristic of the species of interest.

The term “isolated” for the purposes of the present invention designates a biological material (e.g., nucleic acid or protein) that has been removed from its original environment (the environment in which it is naturally present).

For example, a polynucleotide present in the natural state in a plant or an animal is not isolated. The same polynucleotide is “isolated” if it is separated from the adjacent nucleic acids in which it is naturally present. The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, preferably 2 or 3 and preferably 4 or 5 orders of magnitude.

A “nucleic acid” or “polynucleotide” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes but is not limited to cDNA, genomic DNA, plasmid DNA, synthetic DNA, and semi-synthetic DNA. DNA may be linear, circular, or supercoiled.

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”), or any phosphoester anologs thereof, such as phosphorothioates and thioesters, in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” when referring to a polynucleotide will be understood to mean a nucleotide sequence of reduced length relative to the reference nucleic acid and comprising, over the common portion, a nucleotide sequence identical to the reference nucleic acid. Such a nucleic acid fragment according to the invention may be, where appropriate, included in a larger polynucleotide of which it is a constituent. Such fragments comprise, or alternatively consist of, oligonucleotides ranging in length from at least 8, 10, 12, 15, 18, 20 to 25, 30, 40, 50, 70, 80, 100, 200, 500, 1000 or 1500 consecutive nucleotides of a nucleic acid according to the invention.

As used herein, an “isolated nucleic acid fragment” is a polymer of RNA or DNA that is single- or double-stranded, optionally containing synthetic, non-natural or altered nucleotide bases. An isolated nucleic acid fragment in the form of a polymer of DNA may be comprised of one or more segments of cDNA, genomic DNA or synthetic DNA.

A “gene” refers to an assembly of nucleotides that encode a polypeptide, and includes cDNA and genomic DNA nucleic acids. “Gene” also refers to a nucleic acid fragment that expresses a specific protein or polypeptide, optionally including regulatory sequences preceding (5′ noncoding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism A “foreign” gene or “heterologous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Heterologous” DNA refers to DNA not naturally located in the cell, or in a chromosomal site of the cell. Preferably, the heterologous DNA includes a gene or polynucleotides foreign to the cell.

The term “genome” includes chromosomal as well as mitochondrial, chloroplast and viral DNA or RNA.

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength (see Sambrook et al., 1989 infra). Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein (entirely incorporated herein by reference). The conditions of temperature and ionic strength determine the “stringency” of the hybridization. Stringency conditions can be adjusted to screen for moderately similar fragments, such as homologous sequences from distantly related organisms, to highly similar fragments, such as genes that duplicate functional enzymes from closely related organisms. For preliminary screening for homologous nucleic acids, low stringency hybridization conditions, corresponding to a Tm of 55° C., can be used, e.g., 5×SSC, 0.1% SDS, 0.25% milk, and no formamide; or 30% formamide, 5×SSC, 0.5% SDS). Moderate stringency hybridization conditions correspond to a higher Tm, e.g., 40% formamide, with 5× or 6×SCC. High stringency hybridization conditions correspond to the highest Tm, e.g., 50% formamide, 5× or 6×SCC. Hybridization requires that the two nucleic acids contain complementary sequences, although depending on the stringency of the hybridization, mismatches between bases are possible.

The term “complementary” is used to describe the relationship between nucleotide bases that are capable of hybridizing to one another. For example, with respect to DNA, adenosine is complementary to thymine and cytosine is complementary to guanine. Accordingly, the instant invention also includes isolated nucleic acid fragments that are complementary to the complete sequences as disclosed or used herein as well as those substantially similar nucleic acid sequences.

As used herein, the term “oligonucleotide” refers to a nucleic acid, generally of at least 18 nucleotides, that is hybridizable to a genomic DNA molecule, a cDNA molecule, a plasmid DNA or an mRNA molecule. Oligonucleotides can be labeled, e.g., with ³²P-nucleotides or nucleotides to which a label, such as biotin, has been covalently conjugated. A labeled oligonucleotide can be used as a probe to detect the presence of a nucleic acid. Oligonucleotides (one or both of which may be labeled) can be used as PCR primers, either for cloning full length or a fragment of a nucleic acid, or to detect the presence of a nucleic acid. An oligonucleotide can also be used to form a triple helix with a DNA molecule. Generally, oligonucleotides are prepared synthetically, preferably on a nucleic acid synthesizer. Accordingly, oligonucleotides can be prepared with non-naturally occurring phosphoester analog bonds, such as thioester bonds, etc.

A “primer” is an oligonucleotide that hybridizes to a target nucleic acid sequence to create a double stranded nucleic acid region that can serve as an initiation point for DNA synthesis under suitable conditions. Such primers may be used in a polymerase chain reaction.

“Polymerase chain reaction” is abbreviated PCR and means an in vitro method for enzymatically amplifying specific nucleic acid sequences. PCR involves a repetitive series of temperature cycles with each cycle comprising three stages: denaturation of the template nucleic acid to separate the strands of the target molecule, annealing a single stranded PCR oligonucleotide primer to the template nucleic acid, and extension of the annealed primer(s) by DNA polymerase. PCR provides a means to detect the presence of the target molecule and, under quantitative or semi-quantitative conditions, to determine the relative amount of that target molecule within the starting pool of nucleic acids.

“Reverse transcription-polymerase chain reaction” is abbreviated RT-PCR and means an in vitro method for enzymatically producing a target cDNA molecule or molecules from an RNA molecule or molecules, followed by enzymatic amplification of a specific nucleic acid sequence or sequences within the target cDNA molecule or molecules as described above. RT-PCR also provides a means to detect the presence of the target molecule and, under quantitative or semi-quantitative conditions, to determine the relative amount of that target molecule within the starting pool of nucleic acids.

A DNA “coding sequence” is a double-stranded DNA sequence that is transcribed and translated into a polypeptide in a cell in vitro or in vivo when placed under the control of appropriate regulatory sequences. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from mRNA, genomic DNA sequences, and even synthetic DNA sequences. If the coding sequence is intended for expression in a eukaryotic cell, a polyadenylation signal and transcription termination sequence will usually be located 3′ to the coding sequence.

“Open reading frame” is abbreviated ORF and means a length of nucleic acid sequence, either DNA, cDNA or RNA, that comprises a translation start signal or initiation codon such as an ATG or AUG, and a termination codon and can be potentially translated into a polypeptide sequence.

The term “head-to-head” is used herein to describe the orientation of two polynucleotide sequences in relation to each other. Two polynucleotides are positioned in a head-to-head orientation when the 5′ end of the coding strand of one polynucleotide is adjacent to the 5′ end of the coding strand of the other polynucleotide, whereby the direction of transcription of each polynucleotide proceeds away from the 5′ end of the other polynucleotide. The term “head-to-head” may be abbreviated (5′)-to-(5′) and may also be indicated by the symbols (←→) or (3′←5′5′→3′).

The term “tail-to-tail” is used herein to describe the orientation of two polynucleotide sequences in relation to each other. Two polynucleotides are positioned in a tail-to-tail orientation when the 3′ end of the coding strand of one polynucleotide is adjacent to the 3′ end of the coding strand of the other polynucleotide, whereby the direction of transcription of each polynucleotide proceeds toward the other polynucleotide. The term “tail-to-tail” may be abbreviated (3′)-to-(3′) and may also be indicated by the symbols (→←) or (5′→3′3′←5′).

The term “head-to-tail” is used herein to describe the orientation of two polynucleotide sequences in relation to each other. Two polynucleotides are positioned in a head-to-tail orientation when the 5′ end of the coding strand of one polynucleotide is adjacent to the 3′ end of the coding strand of the other polynucleotide, whereby the direction of transcription of each polynucleotide proceeds in the same direction as that of the other polynucleotide. The term “head-to-tail” may be abbreviated (5′)-to-(3′) and may also be indicated by the symbols (→→) or (5′→3′5′→3′).

The term “downstream” refers to a nucleotide sequence that is located 3′ to reference nucleotide sequence. In particular, downstream nucleotide sequences generally relate to sequences that follow the starting point of transcription. For example, the translation initiation codon of a gene is located downstream of the start site of transcription.

The term “upstream” refers to a nucleotide sequence that is located 5′ to reference nucleotide sequence. In particular, upstream nucleotide sequences generally relate to sequences that are located on the 5′ side of a coding sequence or starting point of transcription. For example, most promoters are located upstream of the start site of transcription.

The terms “restriction endonuclease” and “restriction enzyme” refer to an enzyme that binds and cuts within a specific nucleotide sequence within double stranded DNA.

“Homologous recombination” refers to the insertion of a foreign DNA sequence into another DNA molecule, e.g., insertion of a vector in a chromosome. Preferably, the vector targets a specific chromosomal site for homologous recombination. For specific homologous recombination, the vector will contain sufficiently long regions of homology to sequences of the chromosome to allow complementary binding and incorporation of the vector into the chromosome. Longer regions of homology, and greater degrees of sequence similarity, may increase the efficiency of homologous recombination.

Methods known in the art may be used to propagate a polynucleotide according to the invention. Once a suitable host system and growth conditions are established, recombinant expression vectors can be propagated and prepared in quantity. As described herein, the expression vectors which can be used include, but are not limited to, the following vectors or their derivatives: human or animal viruses such as vaccinia virus or adenovirus; insect viruses such as baculovirus; yeast vectors; bacteriophage vectors (e.g., lambda), and plasmid and cosmid DNA vectors, to name but a few.

A “vector” is any means for the cloning of and/or transfer of a nucleic acid into a host cell. A vector may be a replicon to which another DNA segment may be attached so as to bring about the replication of the attached segment. A “replicon” is any genetic element (e.g., plasmid, phage, cosmid, chromosome, virus) that functions as an autonomous unit of DNA replication in vivo, i.e., capable of replication under its own control. The term “vector” includes both viral and nonviral means for introducing the nucleic acid into a cell in vitro, ex vivo or in vivo. A large number of vectors known in the art may be used to manipulate nucleic acids, incorporate response elements and promoters into genes, etc. Possible vectors include, for example, plasmids or modified viruses including, for example bacteriophages such as lambda derivatives, or plasmids such as PBR322 or pUC plasmid derivatives, or the Bluescript vector. For example, the insertion of the DNA fragments corresponding to response elements and promoters into a suitable vector can be accomplished by ligating the appropriate DNA fragments into a chosen vector that has complementary cohesive termini. Alternatively, the ends of the DNA molecules may be enzymatically modified or any site may be produced by ligating nucleotide sequences (linkers) into the DNA termini. Such vectors may be engineered to contain selectable marker genes that provide for the selection of cells that have incorporated the marker into the cellular genome. Such markers allow identification and/or selection of host cells that incorporate and express the proteins encoded by the marker.

Viral vectors, and particularly retroviral vectors, have been used in a wide variety of gene delivery applications in cells, as well as living animal subjects. Viral vectors that can be used include, but are not limited, to retrovirus, adeno-associated virus, pox, baculovirus, vaccinia, herpes simplex, Epstein-Barr, adenovirus, geminivirus, and caulimovirus vectors. Non-viral vectors include, but are not limited to, plasmids, liposomes, electrically charged lipids (cytofectins), DNAprotein complexes, and biopolymers. In addition to a nucleic acid, a vector may also comprise one or more regulatory regions, and/or selectable markers useful in selecting, measuring, and monitoring nucleic acid transfer results (transfer to which tissues, duration of expression, etc.).

The term “plasmid” refers to an extra chromosomal element often carrying a gene that is not part of the central metabolism of the cell, and usually in the form of circular double-stranded DNA molecules. Such elements may be autonomously replicating sequences, genome integrating sequences, phage or nucleotide sequences, linear, circular, or supercoiled, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

A “cloning vector” is a “replicon,” which is a unit length of a nucleic acid, preferably DNA, that replicates sequentially and which comprises an origin of replication, such as aplasmid, phage or cosmid, to which another nucleic acid segment may be attached so as to bring about the replication of the attached segment. Cloning vectors may be capable of replication in one cell type and expression in another (“shuttle vector”).

The term “transfection” means the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

“Transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “genetic region” will refer to a region of a nucleic acid molecule or a nucleotide sequence that comprises a gene encoding a polypeptide.

In addition, the recombinant vector comprising a polynucleotide according to the invention may include, one or more origins for replication in the cellular hosts in which their amplification or their expression is sought, markers or selectable markers.

The term “selectable marker” means an identifying factor, usually an antibiotic or chemical resistance gene, that is able to be selected for based upon the marker gene's effect, i.e., resistance to an antibiotic, resistance to a herbicide, colorimetric markers, enzymes, fluorescent markers, and the like, wherein the effect is used to track the inheritance of a nucleic acid of interest and/or to identify a cell or organism that has inherited the nucleic acid of interest. Examples of selectable marker genes known and used in the art include, but are not limited to: genes providing resistance to ampicillin, streptomycin, gentamycin, kanamycin, hygromycin, bialaphos herbicide, sulfonamide, glyphosate and the like; and genes that are used as phenotypic markers, i.e., anthocyanin regulatory genes, isopentanyl transferase gene, and the like.

The term “reporter gene” means a nucleic acid encoding an identifying factor that is able to be identified based upon the reporter gene's effect, wherein the effect is used to track the inheritance of a nucleic acid of interest, to identify a cell or organism that has inherited the nucleic acid of interest, and/or to measure gene expression induction or transcription. Examples of reporter genes known and used in the art include, but are not limited to: luciferase (Luc), green fluorescent protein (GFP), chloramphenicol acetyltransferase (CAT), β-galactosidase (LacZ), β-glucuronidase (Gus), and the like. Selectable marker genes may also be considered reporter genes.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters that cause a gene to be expressed in most cell types at most times are commonly referred to as “constitutive promoters”. Promoters that cause a gene to be expressed in a specific cell type are commonly referred to as “cell-specific promoters” or “tissue-specific promoters”. Promoters that cause a gene to be expressed at a specific stage of development or cell differentiation are commonly referred to as “developmentally-specific promoters” or “cell differentiation-specific promoters”. Promoters that are induced and cause a gene to be expressed following exposure or treatment of the cell with an agent, biological molecule, chemical, ligand, light, or the like that induces the promoter are commonly referred to as “inducible promoters” or “regulatable promoters”. It is further recognized that since in most cases the exact boundaries of regulatory sequences have not been completely defined, DNA fragments of different lengths may have identical promoter activity.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease Si), as well as protein binding domains (consensus sequences) responsible for the binding of RNA polymerase or transcription factors.

A coding sequence is “under the control” of transcriptional and translational control sequences in a cell when RNA polymerase transcribes the coding sequence into mRNA, which is then RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

The term “response element” means one or more cis-acting DNA elements which confer responsiveness on a promoter mediated through interaction with the DNA-binding domain of a polypeptide. This DNA element may be, for example,palindromic (perfect or imperfect) in its sequence or composed of sequence motifs or half sites separated by a variable number of nucleotides. The half sites can be similar or identical and arranged as either direct or inverted repeats or as a single half site or multimers of adjacent half sites in tandem. The response element may comprise a minimal promoter isolated from different organisms depending upon the nature of the cell or organism into which the response element will be incorporated. The DNA binding domain of the first hybrid protein binds, in the presence or absence of a ligand, to the DNA sequence of a response element to initiate or suppress transcription of downstream gene(s) under the regulation of this response element. Examples of DNA sequences for response elements of natural ecdysone receptor polypeptides include, but are not limited to: RRGG/TTCANTGAC/ACYY (see Cherbas L., et al., (1991), Genes Dev. 5, 120-131); AGGTCAN(n)AGGTCA, where N(n) can be one or more spacer nucleotides (see D'Avino P P., et al., (1995) Mol. Cell. Endocrinol. 113:1-9); and GGGTTGAATGAATTT (see Antoniewski C., et al., (1994) Mol. Cell Biol. 14:4465-4474).

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The term “expression,” as used herein, refers to the transcription and stable accumulation of sense (mRNA) or antisense RNA derived from a nucleic acid or polynucleotide. Expression may also refer to translation of mRNA into a protein or polypeptide.

The terms “cassette,” “expression cassette” and “gene expression cassette” refer to a segment of DNA that can be inserted into a nucleic acid or polynucleotide at specific restriction sites or by homologous recombination. The segment of DNA comprises a polynucleotide that encodes a polypeptide of interest, and the cassette and restriction sites are designed to ensure insertion of the cassette in the proper reading frame for transcription and translation. “Transformation cassette” refers to a specific vector comprising a polynucleotide that encodes a polypeptide of interest and having elements in addition to the polynucleotide that facilitate transformation of a particular host cell. Cassettes, expression cassettes, gene expression cassettes and transformation cassettes of the invention may also comprise elements that allow for enhanced expression of a polynucleotide encoding a polypeptide of interest in a host cell. These elements may include, but are not limited to: a promoter, a minimal promoter, an enhancer, a response element, a terminator sequence, a polyadenylation sequence, and the like.

For purposes of this invention, the term “gene switch” refers to the combination of a, response element associated with a promoter, and an EcR based system which, in the presence of one or more ligands, modulates the expression of a gene into which the response element and promoter are incorporated.

The terms “modulate” and “modulates” mean to induce, reduce or inhibit nucleic acid or gene expression, resulting in the respective induction, reduction or inhibition of protein or polypeptide production.

“Regulatory region” means a nucleic acid sequence which regulates the expression of a second nucleic acid sequence. A regulatory region may include sequences which are naturally responsible for expressing a particular nucleic acid (a homologous region) or may include sequences of a different origin that are responsible for expressing different proteins or even synthetic proteins (a heterologous region). In particular, the sequences can be sequences of prokaryotic, eukaryotic, or viral genes or derived sequences that stimulate or repress transcription of a gene in a specific or non-specific manner and in an inducible or non-inducible manner. Regulatory regions include, but are not limited to, origins of replication, RNA splice sites, promoters, enhancers, transcriptional termination sequences, and signal sequences which direct the polypeptide into the secretory pathways of the target cell.

A regulatory region from a “heterologous source” is a regulatory region that is not naturally associated with the expressed nucleic acid. Included among the heterologous regulatory regions are regulatory regions from a different species, regulatory regions from a different gene, hybrid regulatory sequences, and regulatory sequences which do not occur in nature, but which are designed by one having ordinary skill in the art.

The plasmids or vectors according to the invention may further comprise at least one promoter suitable for driving expression of a gene in a host cell. The term “expression vector” means a vector, plasmid or vehicle designed to enable the expression of an inserted nucleic acid sequence following transformation into the host, the cloned gene, i.e., the inserted nucleic acid sequence, is usually placed under the control of control elements such as a promoter, a minimal promoter, an enhancer, or the like.

The terms “3′ non-coding sequences” or “3′ untranslated region (UTR)” refer to DNA sequences located downstream (3′) of a coding sequence and may comprise polyadenylation [poly(A)]recognition sequences and other sequences encoding regulatory signals capable of affecting mRNA processing or gene expression. The polyadenylation signal is usually characterized by affecting the addition of polyadenylic acid tracts to the 3′ end of the mRNA precursor.

“RNA transcript” refers to the product resulting from RNA polymerase-catalyzed transcription of a DNA sequence. When the RNA transcript is a perfect complementary copy of the DNA sequence, it is referred to as the primary transcript or it may be a RNA sequence derived from post-transcriptional processing of the primary transcript and is referred to as the mature RNA. “Messenger RNA (mRNA)” refers to the RNA that is without introns and that can be translated into protein by the cell. “cDNA” refers to a double-stranded DNA that is complementary to and derived from mRNA. “Sense” RNA refers to RNA transcript that includes the mRNA and so can be translated into protein by the cell. “Antisense RNA” refers to a RNA transcript that is complementary to all or part of a target primary transcript or mRNA and that blocks the expression of a target gene. The complementarity of an antisense RNA may be with any part of the specific gene transcript, i.e., at the 5′ non-coding sequence, 3′ non-coding sequence, or the coding sequence. “Functional RNA” refers to antisense RNA, ribozyme RNA, or other RNA that is not translated yet has an effect on cellular processes.

A “polypeptide” is a polymeric compound comprised of covalently linked amino acid residues. Amino acids have the following general structure:

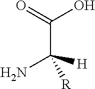

Amino acids are classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

A “protein” is a polypeptide that performs a structural or functional role in a living cell.

An “isolated polypeptide” or “isolated protein” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

“Fragment” of a polypeptide according to the invention will be understood to mean a polypeptide whose amino acid sequence is shorter than that of the reference polypeptide and which comprises, over the entire portion with these reference polypeptides, an identical amino acid sequence. Such fragments may, where appropriate, be included in a larger polypeptide of which they are a part. Such fragments of a polypeptide according to the invention may have a length of 10, 15, 20, 30 to 40, 50, 100, 200 or 300 amino acids.

A “variant” of a polypeptide or protein is any analogue, fragment, derivative, or mutant which is derived from a polypeptide or protein and which retains at least one biological property of the polypeptide or protein. Different variants of the polypeptide or protein may exist in nature. These variants may be allelic variations characterized by differences in the nucleotide sequences of the structural gene coding for the protein, or may involve differential splicing or post-translational modification. The skilled artisan can produce variants having single or multiple amino acid substitutions, deletions, additions, or replacements. These variants may include, inter alia: (a) variants in which one or more amino acid residues are substituted with conservative or non-conservative amino acids, (b) variants in which one or more amino acids are added to the polypeptide or protein, (c) variants in which one or more of the amino acids includes a substituent group, and (d) variants in which the polypeptide or protein is fused with another polypeptide such as serum albumin. The techniques for obtaining these variants, including genetic (suppressions, deletions, mutations, etc.), chemical, and enzymatic techniques, are known to persons having ordinary skill in the art. A variant polypeptide preferably comprises at least about 14 amino acids.

A “heterologous protein” refers to a protein not naturally produced in the cell.

A “mature protein” refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

The term “signal peptide” refers to an amino terminal polypeptide preceding the secreted mature protein. The signal peptide is cleaved from and is therefore not present in the mature protein. Signal peptides have the function of directing and translocating secreted proteins across cell membranes. Signal peptide is also referred to as signal protein.

A “signal sequence” is included at the beginning of the coding sequence of a protein to be expressed on the surface of a cell. This sequence encodes a signal peptide, N-terminal to the mature polypeptide that directs the host cell to translocate the polypeptide. The term “translocation signal sequence” is used herein to refer to this sort of signal sequence. Translocation signal sequences can be found associated with a variety of proteins native to eukaryotes and prokaryotes, and are often functional in both types of organisms.

The term “homology” refers to the percent of identity between two polynucleotide or two polypeptide moieties. The correspondence between the sequence from one moiety to another can be determined by techniques known to the art. For example, homology can be determined by a direct comparison of the sequence information between two polypeptide molecules by aligning the sequence information and using readily available computer programs. Alternatively, homology can be determined by hybridization of polynucleotides under conditions that form stable duplexes between homologous regions, followed by digestion with single-stranded-specific nuclease(s) and size determination of the digested fragments.

As used herein, the term “homologous” in all its grammatical forms and spelling variations refers to the relationship between proteins that possess a “common evolutionary origin,” including proteins from superfamilies (e.g., the immunoglobulin superfamily) and homologous proteins from different species (e.g., myosin light chain, etc.) (Reeck et al., 1987, Cell 50:667.). Such proteins (and their encoding genes) have sequence homology, as reflected by their high degree of sequence similarity.

Accordingly, the term “sequence similarity” in all its grammatical forms refers to the degree of identity or correspondence between nucleic acid or amino acid sequences of proteins that may or may not share a common evolutionary origin (see Reeck et al. (1987) Cell 50:667). As used herein, the term “homologous” in all its grammatical forms and spelling variations refers to the relationship between proteins that possess a “common evolutionary origin,” including proteins from superfamilies and homologous proteins from different species (Reeck et al., supra). Such proteins (and their encoding genes) have sequence homology, as reflected by their high degree of sequence similarity. However, in common usage and in the instant application, the term “homologous,” when modified with an adverb such as “highly,” may refer to sequence similarity and not a common evolutionary origin.

In a specific embodiment, two DNA sequences are “substantially homologous” or “substantially similar” when at least about 50% (preferably at least about 75%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Sambrook et al., 1989, supra.

As used herein, “substantially similar” refers to nucleic acid fragments wherein changes in one or more nucleotide bases results in substitution of one or more amino acids, but do not affect the functional properties of the protein encoded by the DNA sequence. “Substantially similar” also refers to nucleic acid fragments wherein changes in one or more nucleotide bases does not affect the ability of the nucleic acid fragment to mediate alteration of gene expression by antisense or co-suppression technology. “Substantially similar” also refers to modifications of the nucleic acid fragments of the instant invention such as deletion or insertion of one or more nucleotide bases that do not substantially affect the functional properties of the resulting transcript. It is therefore understood that the invention encompasses more than the specific exemplary sequences. Each of the proposed modifications is well within the routine skill in the art, as is determination of retention of biological activity of the encoded products.

Moreover, the skilled artisan recognizes that substantially similar sequences encompassed by this invention are also defined by their ability to hybridize, under stringent conditions (0.1×SSC, 0.1% SDS, 65° C. and washed with 2×SSC, 0.1% SDS followed by 0.1×SSC, 0.1% SDS), with the sequences exemplified herein. Substantially similar nucleic acid fragments of the instant invention are those nucleic acid fragments whose DNA sequences are at least 70% identical to the DNA sequence of the nucleic acid fragments reported herein. Preferred substantially nucleic acid fragments of the instant invention are thus nucleic acid fragments whose DNA sequences are at least 80% identical to the DNA sequence of the nucleic acid fragments reported herein. More preferred nucleic acid fragments are at least 90% identical to the DNA sequence of the nucleic acid fragments reported herein. Even more preferred are nucleic acid fragments that are at least 95% identical to the DNA sequence of the nucleic acid fragments reported herein.

Two amino acid sequences are “substantially homologous” or “substantially similar” when greater than about 40% of the amino acids are identical, or greater than 60% are similar (functionally identical). Preferably, the similar or homologous sequences are identified by alignment using, for example, the GCG (Genetics Computer Group, Program Manual for the GCG Package, Version 7, Madison, Wis.) pileup program.

The term “corresponding to” is used herein to refer to similar or homologous sequences, whether the exact position is identical or different from the molecule to which the similarity or homology is measured. A nucleic acid or amino acid sequence alignment may include spaces. Thus, the term “corresponding to” refers to the sequence similarity, and not the numbering of the amino acid residues or nucleotide bases.

A “substantial portion” of an amino acid or nucleotide sequence comprises enough of the amino acid sequence of a polypeptide or the nucleotide sequence of a gene to putatively identify that polypeptide or gene, either by manual evaluation of the sequence by one skilled in the art, or by computer-automated sequence comparison and identification using algorithms such as BLAST (Basic Local Alignment Search Tool; Altschul, S. F., et al., (1993) J. Mol. Biol. 215:403-410; see also www ncbi.nlm.nih.gov/BLAST/). In general, a sequence of ten or more contiguous amino acids or thirty or more nucleotides is necessary in order to putatively identify a polypeptide or nucleic acid sequence as homologous to a known protein or gene. Moreover, with respect to nucleotide sequences, gene specific oligonucleotide probes comprising 20-30 contiguous nucleotides may be used in sequence-dependent methods of gene identification (e.g., Southern hybridization) and isolation (e.g., in situ hybridization of bacterial colonies or bacteriophage plaques). In addition, short oligonucleotides of 12-15 bases may be used as amplification primers in PCR in order to obtain a particular nucleic acid fragment comprising the primers. Accordingly, a “substantial portion” of a nucleotide sequence comprises enough of the sequence to specifically identify and/or isolate a nucleic acid fragment comprising the sequence.

The term “percent identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in: COMPUTATIONAL MOLECULAR BIOLOGY (Lesk, A. M., ed.) Oxford University Press, New York (1988); Biocomputing: Informatics and Genome Projects (Smith, D. W., ed.) Academic Press, New York (1993); COMPUTER ANALYSIS OF SEQUENCE DATA, PART I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR Inc., Madison, Wis.). Multiple alignment of the sequences may be performed using the Clustal method of alignment (Higgins and Sharp (1989) CABIOS. 5:151-153) with the default parameters (GAP PENALTY=10, GAP LENGTH PENALTY=10). Default parameters for pairwise alignments using the Clustal method may be selected: KTUPLE 1, GAP PENALTY=3, WINDOW=5 and DIAGONALS SAVED=5.

The term “sequence analysis software” refers to any computer algorithm or software program that is useful for the analysis of nucleotide or amino acid sequences. “Sequence analysis software” may be commercially available or independently developed. Typical sequence analysis software will include but is not limited to the GCG suite of programs (Wisconsin Package Version 9.0, Genetics Computer Group (GCG), Madison, Wis.), BLASTP, BLASTN, BLASTX (Altschul et al., J. Mol. Biol. 215:403-410 (1990), and DNASTAR (DNASTAR, Inc. 1228 S. Park St. Madison, Wis. 53715 USA). Within the context of this application it will be understood that where sequence analysis software is used for analysis, that the results of the analysis will be based on the “default values” of the program referenced, unless otherwise specified. As used herein “default values” will mean any set of values or parameters which originally load with the software when first initialized.

“Synthetic genes” can be assembled from oligonucleotide building blocks that are chemically synthesized using procedures known to those skilled in the art. These building blocks are ligated and annealed to form gene segments that are then enzymatically assembled to construct the entire gene. “Chemically synthesized,” as related to a sequence of DNA, means that the component nucleotides were assembled in vitro. Manual chemical synthesis of DNA may be accomplished using well established procedures, or automated chemical synthesis can be performed using one of a number of commercially available machines. Accordingly, the genes can be tailored for optimal gene expression based on optimization of nucleotide sequence to reflect the codonbias of the host cell. The skilled artisan appreciates the likelihood of successful gene expression if codon usage is biased towards those codons favored by the host. Determination of preferred codons can be based on a survey of genes derived from the host cell where sequence information is available.

### A. The EcR-Based Gene Switch

The invention provides an ecdysone-receptor (EcR)-based gene switch system for controlled expression of phenotypic traits in plants. In particular, the invention provides a system for controlling expression of genes in plants which can be turned “on” or “off” as desired. For example, in some applications, the invention provides control of flowering, disease resistance, flower color, nutritional value, and expression of desirable traits. In some embodiments, for example, it will be desirable to turn flowering off, such as, for example, when growing alfalfa so that the biomass and quality of the alfalfa is increased. In other applications, turning flowering on will be desirable such as for seed harvest.

In certain embodiments, the invention comprises use of an EcR based gene switch to control gene expression in plants (the plants may be monocotyledonous or dicotyledonous). In certain embodiments, the EcR-based gene switch comprises a ligand binding domain derived from an ecdysone receptor polypeptide of Choristoneura fumiferana (Cf) (also known as spruce budworm).

In certain embodiments, the EcR-based gene switch is a heterologous chimeric polypeptide comprised of a transcriptional transactivator domain (AD), a DNA-binding domain (DBD) and an EcR ligand binding domain (LBD). The DBD is characterized by the presence of two cysteine zinc fingers, between which are two amino acid motifs, the P-box and the D-box, which confer specificity for response elements. These domains may be either native, modified, or chimeras of different domains of heterologous receptor proteins. The EcR, like a subset of the nuclear receptor family, also possesses regions responsible for heterodimerization properties. Because the domains of nuclear receptors are modular in nature, the LBD, DBD, and AD may be interchanged.

In another embodiment, the transcription factor comprises an AD, a DBD that recognizes a response element associated with the protein or polynucleotide of interest whose expression is to be modulated; and a Group H nuclear receptor LBD. In certain embodiments, the Group H nuclear receptor LBD comprises one or more substitution mutations.

The DNA binding domain can be any DNA binding domain (DBD) with a known response element, including synthetic and chimeric DNA binding domains, or analogs, combinations, or modifications thereof. In one embodiment, the DNA binding domain is selected from the group consisting of a GAL4 DBD, a LexA DBD, a transcription factor DBD, a Group H nuclear receptor member DBD, a steroid/thyroid hormone nuclear receptor superfamily member DBD, a bacterial LacZ DBD, an EcR DBD and a plant DBD (such as, but not limited to ARFs, a bZIP DBD, plant homeodomain families of DNA binding domains or synthetic DNA-binding proteins generated through directed evolution (e.g., SELEX) as described in Tiwari et al. (2003) Plant Cell, 15:533-543).

The transactivation domain (abbreviated “AD” or “TA”) may be any Group H nuclear receptor member AD, steroid/thyroid hormone nuclear receptor AD, synthetic or chimeric AD, polyglutamine AD, basic or acidic amino acid AD, a VP16 AD, a GAL4 AD, an NF-κB AD, a BP64 AD, a B42 acidic activation domain (B42AD), a p53 transactivtion domain (p53AD), a p65 transactivation domain (p65AD), cREL, a plant transactivation domain such as, but not limited to an EDLL acidic transactivation domain from the AP2/ERF family, a glutamine-rich transactivation domain from ARFs and CONSTANS proteins, a proline-rich domains from a bZIP family (as described in Tiwari, S. B. et al. (2012) Plant J. 70:855-865; Tiwari, S. B. et al. (2003) Plant Cell 15(2):533-543; Sprenger-Haussels, M. and B. Weisshaar (2000) Plant J. 22(1):1-8) or an analog, combination, or modification thereof.

In some embodiments, the EcR-based gene switch is comprised of an AD, DBD and LBD from three different species of organisms. In certain embodiments, the transcriptional transactivator domain is a herpes virus VP16 polypeptide or an SV40 virus transcriptional transactivator domain. In certain embodiments, the DNA-binding domain comprises a Gal4 transcription factor DNA binding polypeptide.

In certain embodiments, an EcR-based gene switch of the invention comprises the substitution mutated CfEcR polypeptide designated herein as Evy (E68V/V184I/Y204E); as shown in SEQ ID NO:2. In certain embodiments, an EcR-based gene switch of the invention comprises the monopartite gene switch polypeptide designated herein as VGEvy (E68V/V184I/Y204E) as shown in SEQ ID NO:1 (numbering of with respect to E68V/V184I/Y204E is with respect to the ligand binding domain shown in SEQ ID NO:2).

In certain embodiments, the invention comprises biologically active polypeptides fragments of VGEvy (E68V/V184I/Y204E (SEQ ID NO:1)) or Evy (E68V/V184I/Y204E (SEQ ID NO:12)) and polypeptides at least 80% identical, at least 85% identical, at least 90% identical, at least 95% identical, at least 97% identical, at least 98% identical, at least 99% identical, or 100% identical to SEQ ID NO:1 or SEQ ID NO:2 provided E68V, V184I, and Y204E are unchanged.

In certain embodiments, the invention comprises polynucleotides encoding VGEvy (E68V/V184I/Y204E (SEQ ID NO:1)) or Evy (E68V/V184I/Y204E (SEQ ID NO:2)) polypeptides, as well as polynucleotides encoding polypeptides at least 80% identical, at least 85% identical, at least 90% identical, at least 95% identical, at least 97% identical, at least 98% identical, at least 99% identical, or 100% identical to SEQ ID NO:1 or SEQ ID NO:2 provided that the coding sequences do not change E68V, V184I, or Y204E.

In some embodiments, the EcR-based gene switch is activated by methoxyfenozide. Methoxyfenozide is a diacylhydrazine (organic chemical) compound which acts as a molt accelerating compound in insects. It is specifically effective against a broad range of lepidopterous (e.g., caterpillar) insect species. Methoxyfenozide is also identified as: 3-methoxy-2-methylbenzoic acid 2-(3,5-dimethylbenzoyl)-2-(1,1-dimethylethyl)hydrazide and as Benzoic acid, 3-methoxy-2-methyl-,2-(3,5-dimethylbenzoyl)-2-(1,1-dimethylethyl) hydrazide. See, for example, Carlson et al., “The chemical and biological properties of methoxyfenozide, a new insecticidal ecdysteroid agonist” Pest Manag. Sci., 57(2):115-119 (February 2001) which is hereby incorporated by reference in its entirety. See also the NCBI (United States National Center for Biotechnology Information (U.S. National Library of Medicine 8600 Rockville Pike, Bethesda MD, 20894 USA)) PubChem Substance Database which contains descriptions of samples, from a variety of sources. The description for methoxyfenozide includes links to additional compositional, safety, structural and supplier information.

It will be understood that any phenotypic trait could be put under the control of an EcR-based gene switch system of the invention. Non-limiting examples of phenotypic traits include stress resistance (abiotic, such as drought, or biotic such as pests and diseases); biofactory traits, such as the production of high value compounds (active pharmaceutical ingredients, biopolymers, etc.) in plants when it is necessary to control the timing of compound accumulation (switching it on before harvesting) because of the fitness cost; tunable traits such as type and intensity of color and aroma in flowers, and controlled accumulation of specific nutrients in fruits and vegetables such as differentiating taste and nutritional value to satisfy different groups of consumers. Timing of expression can be important such that one may select to turn on genes at the proper time in the plant's life. For example, but not by way of limitation, Erwinia amylovora, the causative agent of fire blight in apples and pears, attacks the trees at the time of flowering and will infect the fruit. A gene switch to produce an antibacterial protein or dsRNA against E. amylovora can be induced by applying the chemical ligand at the time of flowering to inhibit fire blight. In another non-limiting example, the ability to change the color of cotton filaments had not been possible due to the inability of being able to control the timing of color expression. With the gene switch of the invention, one may time expression of color in cotton filaments to alter the color of cotton at the time of boll formation.

### B. Additional Gene Switch Systems for Use Alone or in Combination.

In some embodiments of the invention, at least one other gene switch sytem is included. Other gene switches that may be used in the invention may be any gene switch that regulates gene expression by addition or removal of a specific ligand or other gene switch activator (e.g., light, heat, cold, etc). In one embodiment, the gene switch is one in which the level of gene expression is dependent on the level of ligand that is present. Examples of ligand-dependent transcription factor complexes that may be used in the gene switches of the invention include, without limitation, members of the nuclear receptor superfamily activated by their respective ligands (e.g., glucocorticoid, estrogen, progestin, retinoid, ecdysone, and analogs and mimetics thereof) and rTTA activated by tetracycline. In one aspect of the invention, the gene switch is an EcR-based gene switch. Examples of such systems include, without limitation, the systems described in U.S. Pat. Nos. 6,258,603, 7,045,315, U.S. Published Patent Application Nos. 2006/0014711, 2007/0161086, and International Published Application No. WO 01/70816. Examples of chimeric ecdysone receptor systems are described in U.S. Pat. No. 7,091,038, U.S. Published Patent Application Nos. 2002/0110861, 2004/0033600, 2004/0096942, 2005/0266457, and 2006/0100416, and International Published Application Nos. WO 01/70816, WO 02/066612, WO 02/066613, WO 02/066614, WO 02/066615, WO 02/29075, and WO 2005/108617, each of which is incorporated by reference in its entirety.

In one aspect of the invention, the gene switch is an EcR-based gene switch. Examples of such systems include, without limitation, the systems described in: PCT/US2001/009050 (WO 2001/070816); U.S. Pat. Nos. 7,091,038; 7,776,587; 7,807,417; 8,202,718; PCT/US2001/030608 (WO 2002/029075); U.S. Pat. Nos. 8,105,825; 8,168,426; PCT/US2002/005235 (WO 2002/066613); U.S. application Ser. No. 10/468,200 (U.S. Pub. No. 20120167239); PCT/US2002/005706 (WO 2002/066614); U.S. Pat. Nos. 7,531,326; 8,236,556; 8,598,409; PCT/US2002/005090 (WO 2002/066612); U.S. application Ser. No. 10/468,193 (U.S. Pub. No. 20060100416); PCT/US2002/005234 (WO 2003/027266); U.S. Pat. Nos. 7,601,508; 7,829,676; 7,919,269; 8,030,067; PCT/US2002/005708 (WO 2002/066615); U.S. application Ser. No. 10/468,192 (U.S. Pub. No. 20110212528); PCT/US2002/005026 (WO 2003/027289); U.S. Pat. Nos. 7,563,879; 8,021,878; 8,497,093; PCT/US2005/015089 (WO 2005/108617); U.S. Pat. Nos. 7,935,510; 8,076,454; PCT/US2008/011270 (WO 2009/045370); U.S. application Ser. No. 12/241,018 (U.S. Pub. No. 20090136465); PCT/US2008/011563 (WO 2009/048560); U.S. application Ser. No. 12/247,738 (U.S. Pub. No. 20090123441); PCT/US2009/005510 (WO 2010/042189); U.S. application Ser. No. 13/123,129 (U.S. Pub. No. 20110268766); PCT/US2011/029682 (WO 2011/119773); U.S. application Ser. No. 13/636,473 (U.S. Pub. No. 20130195800); PCT/US2012/027515 (WO 2012/122025); and, U.S. application Ser. No. 14/001,943 (U.S. Pub. No. 20140308247), each of which is incorporated by reference in its entirety.

In another aspect of the invention, the gene switch is based on heterodimerization of FK506 binding protein (FKBP) with FKBP rapamycin associated protein (FRAP) and is regulated through rapamycin or its non-immunosuppressive analogs. Examples of such systems, include, without limitation, the ARGENT™ Transcriptional Technology (ARIAD Pharmaceuticals, Cambridge, MA) and the systems described in U.S. Pat. Nos. 6,015,709; 6,117,680; 6,479,653; 6,187,757; and 6,649,595.

In one embodiment, the gene switch comprises a single transcription factor sequence encoding a ligand-dependent transcription factor complex under the control of a switch promoter. The transcription factor sequence may encode a ligand-dependent transcription factor complex that is a naturally occurring or an artificial ligand-dependent transcription factor complex. An artificial transcription factor is one in which the natural sequence of the transcription factor has been altered, e.g., by mutation of the sequence or by the combining of domains from different transcription factors. In one embodiment, the transcription factor comprises a Group H nuclear receptor ligand binding domain. In one embodiment, the Group H nuclear receptor ligand binding domain is from an ecdysone receptor, a ubiquitous receptor (UR), an orphan receptor 1 (OR-1), a steroid hormone nuclear receptor 1 (NER-1), a retinoid X receptor interacting protein-15 (RIP-15), a liver X receptor □ (LXR □), a steroid hormone receptor like protein (RLD-1), a liver X receptor (LXR), a liver X receptor □ (LXR□), a farnesoid X receptor (FXR), a receptor interacting protein 14 (RIP-14), or a farnesol receptor (HRR-1). In another embodiment, the Group H nuclear receptor LBD is from an ecdysone receptor.

### 1. Ecdysone-Based Gene Switch

The EcR and the other Group H nuclear receptors are members of the nuclear receptor superfamily wherein all members are generally characterized by the presence of an amino-terminal transactivation domain (AD, also referred to interchangeably as “TA” or “TD”), optionally fused to a heterodimerization partner (HP) to form a coactivation protein (CAP), a DNA binding domain (DBD), and a LBD fused to the DBD via a hinge region to form a ligand-dependent transcription factor (LTF). As used herein, the term “DNA binding domain” comprises a minimal polypeptide sequence of a DNA binding protein, up to the entire length of a DNA binding protein, so long as the DNA binding domain functions to associate with a particular response element. Members of the nuclear receptor superfamily are also characterized by the presence of four or five domains: A/B, C, D, E, and in some members F (see U.S. Pat. No. 4,981,784 and Evans (1988) Science 240:889). The “A/B” domain corresponds to the transactivation domain, “C” corresponds to the DNA binding domain, “D” corresponds to the hinge region, and “E” corresponds to the ligand binding domain. Some members of the family may also have another transactivation domain on the carboxy-terminal side of the LBD corresponding to “F”.

| | DNA-Binding | | Ligand Binding | |
|---|---|---|---|---|
| Transactivation | Domain | Hinge | Domain | Transactivation |
| A/B | C | D | E | F |

The following polypeptide sequence was reported as a polypeptide sequence of Ecdysone receptor (Ecdysteroid receptor) (20-hydroxy-ecdysone receptor) (20E receptor) (EcRH) (Nuclear receptor subfamily 1 group H member 1) and has the accession number P34021 in Genbank.

Ecdysone receptor (878aa) from Drosophila melanogaster (Fruit fly) (SEQ ID NO:20)

| | |
|---|---|
| 1 | MKRRWSNNGG FMRLPEESSS EVTSSSNGLV LPSGVNMSPS SLDSHDYCDQ DLWLCGNESG |
| 61 | SFGGSNGHGL SQQQQSVITL AMHGCSSTLP AQTTIIPING NANGNGGSTN GQYVPGATNL |
| 121 | GALANGMLNG GFNGMQQQIQ NGHGLINSTT PSTPTTPLHL QQNLGGAGGG GIGGMGILHH |
| 181 | ANGTPNGLIG VVGGGGGVGL GVGGGGVGGL GEQHTPRSDS VNSISSGRDD LSPSSSLNGY |
| 241 | SANESCDAKK SKKGPAPRVQ EELCLVCGDR ASGYHYNALT CEGCKGFFRR SVTKSAVYCC |
| 301 | KFGRACEMDM YMRRKCQECR LKKCLAVGGER PECVVPENQC AMKRREKKAQ KEKDKMTTSP |
| 361 | SSQHGGNGSL ASGGGQDFVK KEILDLMTCE PPQHATIPLL PDEILAKCQA RNIPSLTYNQ |
| 421 | LAVIYKLIWY QDGYEQPSEE DLRRIMSQPD ENESQTDVSF RHITEITILT VQLIVEFAKG |
| 481 | LPAFTKIPQE DQITLLKACS SEVMMLRMAR RYDHSSDSIF FANNRSYTRD SYKMAGMADN |
| 541 | IEDLLHFCRQ MFSMKVDNVE YALLTAIVIF SDRPGLEKAQ LVEAIQSYYI DTLRIYILNR |
| 601 | HCGDSMSLVF YAKLLSILTE LRTLGNQNAE MCFSLKLKNR KLPKFLEEIW DVHAIPPSVQ |
| 661 | SHLQITQEEN ERLERAERMR ASVGGAITAG IDCDSASTSA AAAAAQHQPQ PQPQPQPSSL |
| 721 | TQNDSQHQTQ PQLQPQLPPQ LQGQLQPQLQ PQLQTQLQPQ IQPQPQLLPV SAPVPASVTA |
| 781 | PGSLSAVSTS SEYMGGSAAI GPITPATTSS ITAAVTASST TSAVPMGNGV GVGVGVGGNV |
| 841 | SMYANAQTAM ALMGVALHSH QEQLIGGVAV KSEHSTTA |

In one embodiment, the ecdysone receptor ligand binding domain is selected from the group consisting of an invertebrate ecdysone receptor ligand binding domain, an Arthropod ecdysone receptor ligand binding domain, a Lepidopteran ecdysone receptor ligand binding domain, a Dipteran ecdysone receptor ligand binding domain, an Orthopteran ecdysone receptor ligand binding domain, a Homopteran ecdysone receptor ligand binding domain, a Hemipteran ecdysone receptor ligand binding domain, a spruce budworm Choristoneura fumiferana EcR ecdysone receptor ligand binding domain, a beetle Tenebrio molitor ecdysone receptor ligand binding domain, a Omphisa fuscidentalis (Bamboo caterpillar) ecdysone receptor ligand, a Locusta migratoria (locust) ecdysone receptor ligand, a Manduca sexta ecdysone receptor ligand binding domain, a Heliothies virescens ecdysone receptor ligand binding domain, a midge Chironomus tentans ecdysone receptor ligand binding domain, a silk moth Bombyx mori ecdysone receptor ligand binding domain, a squinting bush brown Bicyclus anynana ecdysone receptor ligand binding domain, a buckeye Junonia coenia ecdysone receptor ligand binding domain, a fruit fly Drosophila melanogaster ecdysone receptor ligand binding domain, a mosquito Aedes aegypti ecdysone receptor ligand binding domain, a blowfly Lucilia capitata ecdysone receptor ligand binding domain, a blowfly Lucilia cuprina ecdysone receptor ligand binding domain, a blowfly Calliphora vicinia ecdysone receptor ligand binding domain, a Mediterranean fruit fly Ceratitis capitata ecdysone receptor ligand binding domain, a locust Locusta migratoria ecdysone receptor ligand binding domain, an aphid Myzus persicae ecdysone receptor ligand binding domain, a fiddler crab Celuca pugilator ecdysone receptor ligand binding domain, an ixodid tick Amblyomma americanum ecdysone receptor ligand binding domain, a whitefly Bamecia argentifoli ecdysone receptor ligand binding domain and a leafhopper Nephotetix cincticeps ecdysone receptor ligand binding domain.

In another embodiment, the ecdysone receptor ligand binding domain is the Choristoneura fumiferana ecdysone receptor ligand binding domain, for which the amino acid sequence is set forth in SEQ ID NO:19.

In another embodiment, the ecdysone receptor ligand binding domain is an analog of the Choristoneura fumiferana ecdysone receptor ligand binding domain that retains at least 80%, 85%, 90%, 95%, 96%, 97%, 98% 99% or 100% of the in vitro Christoneura fumiferana ecdysone receptor ligand binding activity of the Choristoneura fumiferana ecdysone receptor ligand binding domain. In vitro ecdysone receptor ligand binding assays are well known to those of ordinary skill in the art. For example, see WO 02/066612.

In another embodiment, the ecdysone receptor ligand binding domain analog is an ecdysone receptor ligand binding domain disclosed in WO 02/066612, US 2006/0100416, WO 05/108617 and 2005/0266457. In another embodiment, the ecdysone receptor ligand binding domain analog is the V107I/Y127E substitution mutant of SEQ ID NO:19.

In certain embodiments an ecdysone receptor ligand binding domain analog is a monopartite EcR gene switch. In some embodiments the monopartite EcR is derived from Choristoneura fumiferana EcR (CfEcR) containing two substitutions: V395I and Y415E (vy), of CfEcR (CfEcRvy) (Tavva (2008) FEBS J. 275:2161-2176). In some embodiments, the ecdysone receptor gene switch is the same EcR-based gene switch as the first EcR-based gene switch. In some embodiments, a single EcR-based gene switch is used to regulate inducible expression of two different genes of interest, wherein said gene switch and and said two different genes of interest may be part of a single polynucleotide or vector or may be parts of two or three different polynucleotides or vectors.

The DBD may be characterized by the presence of two cysteine zinc fingers, between which are two amino acid motifs, the P-box and the D-box, which confer specificity for response elements. These domains may be either native, modified, or chimeras of different domains of heterologous receptor proteins. The EcR, like a subset of the nuclear receptor family, also possesses less well-defined regions responsible for heterodimerization properties. Because the domains of nuclear receptors are modular in nature, the LBD, DBD, and AD may be interchanged.

In another embodiment, the transcription factor comprises an AD, a DBD that recognizes a response element associated with the protein or polynucleotide of interest whose expression is to be modulated; and a Group H nuclear receptor LBD. In certain embodiments, the Group H nuclear receptor LBD comprises a substitution mutation.

The DNA binding domain can be any DNA binding domain (DBD) with a known response element, including synthetic and chimeric DNA binding domains, or analogs, combinations, or modifications thereof. In one embodiment, the DNA binding domain is selected from the group consisting of a GAL4 DBD, a LexA DBD, a transcription factor DBD, a Group H nuclear receptor member DBD, a steroid/thyroid hormone nuclear receptor superfamily member DBD, a bacterial LacZ DBD, an EcR DBD, and a plant DBD (such as, but not limited to ARFs, bZIP, plant homeodomain families of DNA binding domains or synthetic DNA-binding proteins generated through directed evolution (e.g., SELEX) as described in Tiwari et al. (2003) Plant Cell, 15:533-543).

The transactivation domain (abbreviated “AD” or “TA”) may be any Group H nuclear receptor member AD, steroid/thyroid hormone nuclear receptor AD, synthetic or chimeric AD, polyglutamine AD, basic or acidic amino acid AD, a VP16 AD, a GAL4 AD, an NF-κB AD, a BP64 AD, a B42 acidic activation domain (B42AD), a p53 transactivation domain (p53AD), a p65 transactivation domain (p65AD), a plant activation domain such as, but not limited to an EDLL acidic transactivation domain from the AP2/ERF family, a glutamine-rich transactivation domain from ARFs and CONSTANS proteins, a proline-rich transactivation domain from a bZIP family member (as described in Tiwari, S. B. et al. (2012) Plant J. 70:855-865; Tiwari, S. B. et al. (2003) Plant Cell 15(2):533-543; Sprenger-Haussels, M. and B. Weisshaar (2000) Plant J. 22(1):1-8) or an analog, combination, or modification thereof.

The vectors and polynucleotides of the invention may also contain a transcription repression domain such as an AUX/IAA protein with an LxLxL domain, such as those described in Tiwari, S. B. et al. (2004) Plant Cell 16(2):533-543. Examples include, but are not limited to: TELRLGLPG (SEQ ID NO: 37), TELRLGLPE (SEQ ID NO:38), TELCLGLPG (SEQ ID NO:39), TELTLGLPG (SEQ ID NO:40), TELTLALPG (SEQ ID NO:41), TDLRLGLSF (SEQ ID NO:42), TELDLALGL (SEQ ID NO:43), SELELGLGL (SEQ ID NO:44), MELDLGLSL (SEQ ID NO:45), IELGLTLSL (SEQ ID NO:46), IDLGLDLRT (SEQ ID NO:47), VNLSLSLTF (SEQ ID NO:48), KKLELKLGP (SEQ ID NO:49), KKLELRLHR (SEQ ID NO:50), and KRLELRLAP (SEQ ID NO:51). The repression domain may also be an ERF-associated amphiphilic repression (EAR) domain such as a SIERF36, or SIERF.F.1 (Accssion No. SGN-U564952) as described in Upadhyay, R. K. et al. (2014) PLOS One 9(7): e101995. Examples include NtERF3 (IDLDLNLAP) (SEQ ID NO:52), AtERF4 (LDLELNLPP) (SEQ ID NO:53), and AtSUPR (QDLDLELRL) (SEQ ID NO:54).

A transcription repressor domain may be used in conjunction with a constitutively expressed gene to inhibit a trait of interest (e.g., flowering). For example, but not by way of limitation, a plant may be transformed to include a gene that inhibits flowering such as miR156 which produces dsRNA that inhibits SPL which controls flowering in plants. The transformed plant may also comprise a gene switch of the invention that contains a repressor domain such as LxLxL from an AUX/IAA protein. Upon application of an activating chemical ligand, the gene switch binds to the DNA responsive element and the repressor domain represses expression of miR156 and flowering is turned back on.

In one embodiment, the first transcription factor sequence encodes a polypeptide comprising a AD, a DBD that recognizes a response element associated with the gene of interest whose expression is to be controlled; and a Group H nuclear receptor LBD, and the second transcription factor sequence encodes a transcription factor comprising a nuclear receptor LBD selected from a vertebrate retinoid X receptor (RXR), an invertebrate RXR, an ultraspiracle protein (USP), or a chimeric nuclear receptor comprising at least two different nuclear receptor ligand binding domain polypeptide fragments selected from a vertebrate RXR, an invertebrate RXR, and a USP (see WO 01/70816A2 and US2004/0096942A1). The “partner” nuclear receptor ligand binding domain may further comprise a truncation mutation, a deletion mutation, a substitution mutation, or another modification.

In another embodiment, the gene switch comprises a first transcription factor sequence encoding a first polypeptide comprising a nuclear receptor LBD and a DBD that recognizes a response element associated with the sequence of the gene of interest whose expression is to be controlled, and a second transcription factor sequence encoding a second polypeptide comprising an AD and a nuclear receptor LBD, wherein one of the nuclear receptor LBDs is a Group H nuclear receptor LBD. In one embodiment, the first polypeptide is substantially free of an AD and the second polypeptide is substantially free of a DBD. For purposes of the invention, “substantially free” means that the protein in question does not contain a sufficient sequence of the domain in question to provide activation or binding activity.

In one embodiment, a vertebrate RXR LBD is from a human Homo sapiens, mouse Mus musculus, rat Rattus norvegicus, chicken Gallus gallus, pig Sus scrofa domestica, frog Xenopus laevis, zebrafish Danio rerio, tunicate Polyandrocarpa misakiensis, or jellyfish Tripedalia cysophora RXR.

In one embodiment, an invertebrate RXR ligand binding domain is from a locust Locusta migratoria ultraspiracle polypeptide (“LmUSP”), an ixodid tick Amblyomma americanum RXR homolog 1 (“AmaRXR1”), an ixodid tick Amblyomma americanum RXR homolog 2 (“AmaRXR2”), a fiddler crab Celuca pugilator RXR homolog (“CpRXR”), a beetle Tenebrio molitor RXR homolog (“TmRXR”), a honeybee Apis mellifera RXR homolog (“AmRXR”), an aphid Myzus persicae RXR homolog (“MpRXR”), or a non-Dipteran/non-Lepidopteran RXR homolog.

In one embodiment, a chimeric RXR LBD comprises at least two polypeptide fragments selected from a vertebrate species RXR polypeptide fragment, an invertebrate species RXR polypeptide fragment, or a non-Dipteran/non-Lepidopteran invertebrate species RXR homolog polypeptide fragment. A chimeric RXR ligand binding domain for use in the present invention may comprise at least two different species RXR polypeptide fragments, or when the species is the same, the two or more polypeptide fragments may be from two or more different isoforms of the species RXR polypeptide fragment. Such chimeric RXR LBDs are disclosed, for example, in WO 2002/066614.

In one embodiment, a chimeric RXR ligand binding domain comprises at least one vertebrate species RXR polypeptide fragment and one invertebrate species RXR polypeptide fragment.

In another embodiment, a chimeric RXR ligand binding domain comprises at least one vertebrate species RXR polypeptide fragment and one non-Dipteran/non-Lepidopteran invertebrate species RXR homolog polypeptide fragment.

In a specific example, binding of the ligand to the LBD of a Group H nuclear receptor and its nuclear receptor LBD partner enables expression of a sequence of a gene of interest. This mechanism does not exclude the potential for ligand binding to the Group H nuclear receptor (GHNR) or its partner, and the resulting formation of active homodimer complexes (e.g. GHNR+GHNR or partner+partner). Preferably, one or more of the receptor domains is varied producing a hybrid gene switch. Typically, one or more of the three domains, DBD, LBD, and AD, may be chosen from a source different than the source of the other domains so that the hybrid genes and the resulting hybrid proteins are optimized in the plant for transactivating activity, complementary binding of the ligand, and recognition of a specific response element. In addition, the response element itself can be modified or substituted with response elements for other DNA binding protein domains such as the GAL-4 protein from yeast (see Sadowski et al., Nature 335:563 (1988)) or LexA protein from Escherichia coli (see Brent et al., Cell 43:729 (1985)), or synthetic response elements specific for targeted interactions with proteins designed, modified, and selected for such specific interactions (see, for example, Kim et al., Proc. Natl. Acad. Sci. USA, 94:3616 (1997)) to accommodate hybrid receptors. Another advantage of two-hybrid systems is that they allow choice of a promoter used to drive the gene expression according to a desired end result. Such double control may be particularly important, especially when cytotoxic proteins are produced, because both the timing of expression as well as the cells wherein expression occurs may be controlled. When genes, operably linked to a suitable promoter, are introduced into the cells of the plant, expression of the exogenous genes is controlled by the presence of the system of this invention. Promoters may be constitutively or inducibly regulated or may be tissue-specific (that is, expressed only in a particular type of cells) or specific to certain developmental stages of the plant.

In certain embodiments a chimeric protein is used comprising an acidic domain of human Herpes Simplex virus as the transactivation domain, “V” fused to a GAL4 DNA-binding domain derived from Saccharomyces cerevisia, “G,” and a Choristoneura fumiferana ecdysone receptor, “E,” as the ligand binding domain (together, “VGE”). In other embodiments, the ecdysone receptor ligand binding portion of VGE is a E68V\V184I\Y204E (evy) substitution mutant with respect to the wild type EcR portion of the VGE construct (mutations shown underlined below (shown in Figures and discussed herein as “VGEvy”) Numbering of the mutations is based on the EcR portion of the molecule shown below in italics.

| SEQ ID NO: 1 | |
|---|---|
| MAPPTDVSLG DELHLDGEDV AMAHADALDD FDLDMLGDGD SPGPGFTPHD SAPYGALDMA | 60 |
| DFEFEQMFTD ALGIDEYGGK LLGTSRRISG GEFGGMKLLS SIEQACDICR LKKLKCSKEK | 120 |
| PKCAKCLKNN WECRYSPKTK RSPLTRAHLT EVESRLERLE QLFLLIFPRE DLDMILKMDS | 180 |
| LQDIKALLTG LFVQDNVNKD AVTDRLASVE TDMPLTLRQH RISATSSSEE SSNKGQRQLT | 240 |
| VSGGSRRISR PECVVPETQC AMKRKEKKAQ KEKDKLPVST TTVDDHMPPI MQCEPPPPEA | 300 |
| ARIHEVVPRF LSDKLLVTNR QKNIPQLTAN QQFLIARLIW YQDGYEQPSD EDLKRITQTW | 360 |
| QQADDENEES DTPFRQITEM TILTVQLIVE FAKGLPGFAK ISQPDQITLL KACSSEVMML | 420 |
| RVARRYDAAS DSILFANNQA YTRDNYRKAG MAEVIEDLLH FCRCMYSMAL DNIHYALLTA | 480 |
| VVIFSDRPGL EQPQLVEEIQ RYYLNTLRIY ILNQLSGSAR SSVIYGKILS ILSELRTLGM | 560 |
| QNSNMCISLK LKNRKLPPFL EEIWDVADMS HTQPPPILES PTNL | 584 |

### 2. Rapamycin Based Gene Switch

The present invention further provides a gene switch system which utilizes FK506 binding protein as the ligand-dependent transcription factor complex and rapamycin as the ligand. In one embodiment, the construct encoding the gene switch comprises
(a) a first polynucleotide encoding a first chimeric protein which binds to rapamycin or an analog thereof and which comprises at least one FK506-binding protein (FKBP) domain and at least one protein domain heterologous thereto, wherein the FKBP domain comprises a peptide sequence selected from:
   (1) a naturally occurring FKBP
   (2) a variant of a naturally occurring FKBP in which up to 10 amino acid residues have been deleted, inserted, or replaced with substitute amino acids, and
   (3) an FKBP encoded by a DNA sequence which selectively hybridizes to a DNA sequence encoding an FKBP of (1) or (2);
(b) a second polynucleotide encoding a second chimeric protein which forms a complex with both (a) rapamycin or a rapamycin analog and (b) the first chimeric protein, and which comprises at least one FKBP:rapamycin binding (FRB) domain and at least one protein domain heterologous thereto, wherein the FRB domain comprises a peptide sequence selected from:
   (4) a naturally occurring FRB domain,
   (5) a variant of a naturally occurring FRB domain in which up to 10 amino acid residues have been deleted, inserted, or replaced with substitute amino acids, and
   (6) an FRB domain encoded by a DNA sequence which selectively hybridizes to a DNA sequence encoding an FRB of (4) or (5).

In this gene switch system, each of the first polynucleotide and the second polynucleotide are under the control of one or more switch promoters as described elsewhere herein. Furthermore, in certain embodiments, at least one protein domain heterologous to the FKBP and/or FRB domains in the first and second chimeric protein may be one or more “action” or “effector” domains. Effector domains may be selected from a wide variety of protein domains including DNA binding domains, transcription activation domains, cellular localization domains and signaling domains (i.e., domains which are capable upon clustering or multimerization, of triggering cell growth, proliferation, differentiation, apoptosis, gene transcription, etc.).

In certain embodiments, one fusion protein contains at least one DNA binding domain (e.g., a GAL4 or ZFHD1 DNA-binding domain) and another fusion protein contains at least one transcription activation domain (e.g., a VP16 or p65 transcription activation domain). Ligand-mediated association of the fusion proteins represents the formation of a transcription factor complex and leads to initiation of transcription of a target gene linked to a DNA sequence recognized by (i.e., capable of binding with) the DNA-binding domain on one of the fusion proteins. Information regarding the gene expression system as well as the ligand is disclosed in U.S. Pat. Nos. 6,187,757 B1, 6,649,595 B1, 6,509,152 B1, 6,479,653 B1, and 6,117,680 B1.

In other embodiments, the present invention provides a gene switch system which comprises polynucleotides encoding two fusion proteins which self-aggregate in the absence of a ligand, wherein (a) the first fusion protein comprises a conditional aggregation domain which binds to a selected ligand and a transcription activation domain, and (b) the second fusion protein comprising a conditional aggregation domain which binds to a selected ligand and a DNA binding domain, and (c) in the absence of ligand, the cells express a gene operably linked to regulatory DNA to which said DNA binding domain binds. Modified cells comprising the gene switch system are expanded in the presence of the ligand in an amount sufficient for repression of the gene. Ligand removal induces expression of the encoded protein that causes cell death. The nucleic acids encoding the two fusion proteins are under the control of at least one conditional promoter. The gene expression system utilizing conditional aggregation domains is disclosed in U.S. Publication No. 2002/0048792.

### 3. Procaryotic Repressor/Operator Based Gene Switch System

In some embodiments, the present invention provides gene switch system comprising (a) a first polynucleotide coding for a transactivator fusion protein comprising a prokaryotic tetracycline (“tet”) repressor and a eucaryotic transcriptional activator protein domain; and (b) a second polynucleotide coding for a gene of interest, wherein said second polynucleotide is operably linked to a minimal promoter and at least one tet operator sequence. The first polynucleotide coding for a transactivator fusion protein may comprise a switch promoter as described elsewhere herein. The expression of the lethal protein is up-regulated in the absence of tetracycline. (see, e.g., Gossen et al. (1992) Proc. Natl. Acad. Sci. 89: 5547-5551; Gossen et al. (1993) TIBS 18: 471-475; Furth et al. (1994) Proc. Natl. Acad. Sci. 91: 9302-9306; and Shockett et al. (1995) Proc. Natl. Acad. Sci. 92: 6522-6526). The TetO expression system is disclosed in U.S. Pat. No. 5,464,758 B1.

In another embodiment, the gene switch system comprises the lactose (“Lac”) repressor-operator systems from the bacterium Escherichia coli. The gene switch system of the present invention may also comprise (a) a first polynucleotide coding for a transactivator fusion protein comprising a prokaryotic lac I repressor and a eucaryotic transcriptional activator protein domain; and (b) a second polynucleotide coding for a gene of interest, wherein said second polynucleotide is operably linked to a gene switch promoter. In the Lac system, a lac operon is inactivated in the absence of lactose, or synthetic analogs such as isopropyl-b-D-thiogalactoside.

Additional gene switch systems include, but are not limited to, those described in the following: U.S. Pat. No. 7,091,038; WO2004078924; EP1266015; US20010044151; US20020110861; US20020119521; US20040033600; US20040197861; US20040235097; US20060020146; US20040049437; US20040096942; US20050228016; US20050266457; US20060100416; WO2001/70816; WO2002/29075; WO2002/066612; WO2002/066613; WO2002/066614; WO2002/066615; WO2005/108617; U.S. Pat. No. 6,258,603; US20050209283; US20050228016; US20060020146; EP0965644; U.S. Pat. Nos. 7,304,162; 7,304,161; MX234742; KR10-0563143; AU765306; AU2002-248500; and AU2002-306550.

### 4. Other Gene Switches

In another aspect of the invention, gene expression cassettes of the invention incorporate a cumate switch system, which works through the CymR repressor that binds the cumate operator sequences with high affinity. (SparQ™ Cumate Switch, System Biosciences, Inc.) The repression is alleviated through the addition of cumate, a non-toxic small molecule that binds to CymR. This system has a dynamic inducibility, can be finely tuned and is reversible and inducible.

In another aspect of the invention, gene expression cassettes of the invention incorporate a riboswitch, which is a regulatory segment of a messenger RNA molecule that binds an effector, resulting in a change in production of the proteins encoded by the mRNA. An mRNA that contains a riboswitch is directly involved in regulating its own activity in response to the concentrations of its effector molecule. Effectors can be metabolites derived from purine/pyrimidine, amino acid, vitamin, or other small molecule co-factors. These effectors act as ligands for the riboswitch sensor, or aptamer. Breaker, RR. Mol Cell. (2011) 43(6):867-79.

In another aspect of the invention, gene expression cassettes of the invention incorporate the biotin-based gene switch system, in which the bacterial repressor protein TetR is fused to streptavidin, which interacts with the synthetic biotinylation signal AVITAG that is fused to VP16 to activate gene expression. Biotinylation of the AVITAG peptide is regulated by a bacterial biotin ligase BirA, thus enabling ligand responsiveness. Weber et al. (2007) Proc. Natl. Acad. Sci. U.S.A. 104, 2643-2648; Weber et al. (2009) Metabolic Engineering, 11(2):117-124.

Additional gene switch systems which may be used as part of the present invention are well known in the art, including but not limited to those described in Auslander and Fussenegger (2012) Trends in Biotechnology 31(3):155-168, incorporated herein by reference.

### 5. Combination of the Gene Switch Systems

The present invention provides nucleic acid compositions, modified cells, and plants comprising two or more gene switch systems comprising different ligand-dependent transcription factor complexes which are activated by an effective amount of one or more ligands, wherein the two or more gene switch systems comprise a first gene switch and a second gene switch, both of which selectively induce expression of one or more genes of interest, upon binding to one or more ligands. Within the scope of the present invention are any numbers of and/or combinations of gene switch systems.

In certain embodiments, there may be a combination of two or more gene switch systems. In some embodiments, the combination is (1) an EcR-based gene switch and (2) a dual-switch ecdysone receptor based gene expression system. In other embodiments, the combination may be (1) an EcR-based gene switch and (2) a rapamycin based gene switch. Alternatively, the combination of gene switch systems may be two identical rapamycin based gene switch systems disclosed above. Any possible combinations of the gene switch systems are within the scope of the invention.

### C. Ligands

As used herein, the term “ligand,” as applied to gene switches (e.g., EcR based gene switches), describes small and soluble molecules having the capability of activating a gene switch to stimulate expression of a polypeptide encoded therein. The ligand for a ligand-dependent transcription factor complex of the invention binds to the protein complex comprising one or more of the ligand binding domain, the heterodimer partner domain, the DNA binding domain, and the transactivation domain. The choice of ligand to activate the ligand-dependent transcription factor complex depends on the type of the gene switch utilized.

Examples of ligands include, without limitation, an ecdysteroid, such as ecdysone, 20-hydroxyecdysone, ponasterone A, muristerone A, and the like, 9-cis-retinoic acid, synthetic analogs of retinoic acid, N,N′-diacylhydrazines such as those disclosed in U.S. Pat. Nos. 6,013,836; 5,117,057; 5,530,028; and 5,378,726 and U.S. Published Application Nos. 2005/0209283 and 2006/0020146; oxadiazolines as described in U.S. Published Application No. 2004/0171651; dibenzoylalkyl cyanohydrazines such as those disclosed in European Application No. 461,809; N-alkyl-N,N′-diaroylhydrazines such as those disclosed in U.S. Pat. No. 5,225,443; N-acyl-N-alkylcarbonylhydrazines such as those disclosed in European Application No. 234,994; N-aroyl-N-alkyl-N′-aroylhydrazines such as those described in U.S. Pat. No. 4,985,461; amidoketones such as those described in U.S. Published Application No. 2004/0049037; each of which is incorporated herein by reference and other similar materials including 3,5-di-tert-butyl-4-hydroxy-N-isobutyl-benzamide, 8-O-acetylharpagide, oxysterols, 22(R) hydroxycholesterol, 24(S) hydroxycholesterol, 25-epoxycholesterol, T0901317, 5-alpha-6-alpha-epoxycholesterol-3-sulfate (ECHS), 7-ketocholesterol-3-sulfate, famesol, bile acids, 1,1-biphosphonate esters, juvenile hormone III, and the like. Examples of diacylhydrazine ligands useful in the present invention include RG-115819 (3,5-Dimethyl-benzoic acid N-(1-ethyl-2,2-dimethyl-propyl)-N′-(2-methyl-3-methoxy-benzoyl)-hydrazide), RG-115932 ((R)-3,5-Dimethyl-benzoic acid N-(1-tert-butyl-butyl)-N′-(2-ethyl-3-methoxy-benzoyl)-hydrazide), and RG-115830 (3,5-Dimethyl-benzoic acid N-(1-tert-butyl-butyl)-N′-(2-ethyl-3-methoxy-benzoyl)-hydrazide). See, e.g., U.S. patent application Ser. No. 12/155,111, published as US 2009/0163592, and PCT Appl. No. PCT/US2008/006757, both of which are incorporated herein by reference in their entireties.

For example, a ligand for the edysone receptor based gene switch may be selected from any suitable ligands. Both naturally occurring ecdysone or ecdyson analogs (e.g., 20-hydroxyecdysone, muristerone A, ponasterone A, ponasterone B, ponasterone C, 26-iodoponasterone A, inokosterone or 26-mesylinokosterone) and non-steroid inducers may be used as a ligand for gene switch of the present invention. U.S. Pat. No. 6,379,945 B1, describes an insect steroid receptor isolated from Heliothis virescens (“HEcR”) which is capable of acting as a gene switch responsive to both steroid and certain non-steroidal inducers. Non-steroidal inducers have a distinct advantage over steroids, in this and many other systems which are responsive to both steroids and non-steroid inducers, for a number of reasons including, for example: lower manufacturing cost, metabolic stability, absence from insects, plants, or mammals, and environmental acceptability. U.S. Pat. No. 6,379,945 B1 describes the utility of two dibenzoylhydrazines, 1,2-dibenzoyl-1-tert-butyl-hydrazine and tebufenozide (N-(4-ethylbenzoyl)-N′-(3,5-dimethylbenzoyl)-N′-tert-butyl-hydrazine) as ligands for an ecdysone-based gene switch. Also included in the present invention as a ligand are other dibenzoylhydrazines, such as those disclosed in U.S. Pat. No. 5,117,057 B1. Use of tebufenozide as a chemical ligand for the ecdysone receptor from Drosophila melanogaster is also disclosed in U.S. Pat. No. 6,147,282. Additional, non-limiting examples of ecdysone ligands are 3,5-di-tert-butyl-4-hydroxy-N-isobutyl-benzamide, 8-O-acetylharpagide, a 1,2-diacyl hydrazine, an N′-substituted-N,N′-disubstituted hydrazine, a dibenzoylalkyl cyanohydrazine, an N-substituted-N-alkyl-N,N-diaroyl hydrazine, an N-substituted-N-acyl-N-alkyl, carbonyl hydrazine or an N-aroyl-N′-alkyl-N′-aroyl hydrazine. (See U.S. Pat. No. 6,723,531).

In one embodiment, the ligand for an ecdysone based gene switch system is a diacylhydrazine ligand or chiral diacylhydrazine ligand. The ligand used in the gene switch system may be compounds of Formula I

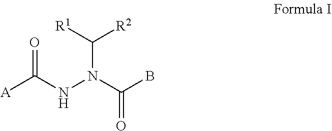

wherein
A is alkoxy, arylalkyloxy or aryloxy;
B is optionally substituted aryl or optionally substituted heteroaryl; and
R¹ and R² are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl;
or pharmaceutically acceptable salts, hydrates, crystalline forms or amorphous forms thereof.

In another embodiment, the ligand may be enantiomerically enriched compounds of Formula II

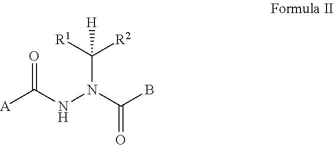

wherein
A is alkoxy, arylalkyloxy, aryloxy, arylalkyl, optionally substituted aryl or optionally substituted heteroaryl;
B is optionally substituted aryl or optionally substituted heteroaryl; and
R¹ and R² are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl;
with the proviso that R¹ does not equal R²;
wherein the absolute configuration at the asymmetric carbon atom bearing R¹ and R² is predominantly S;
or pharmaceutically acceptable salts, hydrates, crystalline forms or amorphous forms thereof.

In certain embodiments, the ligand may be enantiomerically enriched compounds of Formula III

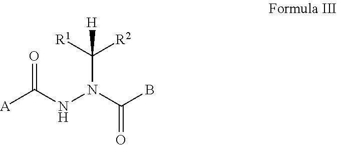

wherein
A is alkoxy, arylalkyloxy, aryloxy, arylalkyl, optionally substituted aryl or optionally substituted heteroaryl;
B is optionally substituted aryl or optionally substituted heteroaryl; and
R¹ and R² are independently optionally substituted alkyl, arylalkyl, hydroxyalkyl, haloalkyl, optionally substituted cycloalkyl, optionally substituted alkenyl, optionally substituted alkynyl, optionally substituted heterocyclo, optionally substituted aryl or optionally substituted heteroaryl;
with the proviso that R¹ does not equal R²;
wherein the absolute configuration at the asymmetric carbon atom bearing R¹ and R² is predominantly R;
or pharmaceutically acceptable salts, hydrates, crystalline forms or amorphous forms thereof.

In one embodiment, a ligand may be (R)-3,5-dimethyl-benzoic acid N-(1-tert-butyl-butyl)-N′-(2-ethyl-3-methoxy-benzoyl)-hydrazide having an enantiomeric excess of at least 95% or a pharmaceutically acceptable salt, hydrate, crystalline form or amorphous form thereof.

The diacylhydrazine ligands of Formula I and chiral diacylhydrazine ligands of Formula II or III, when used with an ecdysone-based gene switch system, provide the means for external temporal regulation of expression of a gene of interest. See U.S. application Ser. No. 12/155,111, published as US 2009/0163592, filed May 29, 2008, which is fully incorporated by reference herein.

The ligands used in the present invention may form salts. The term “salt(s)” as used herein denotes acidic and/or basic salts formed with inorganic and/or organic acids and bases. In addition, when a compound of Formula I, II or III contains both a basic moiety and an acidic moiety, zwitterions (“inner salts”) may be formed and are included within the term “salt(s)” as used herein. Pharmaceutically acceptable (i.e., non-toxic, physiologically acceptable) salts are used, although other salts are also useful, e.g., in isolation or purification steps which may be employed during preparation. Salts of the compounds of Formula I, II or III may be formed, for example, by reacting a compound with an amount of acid or base, such as an equivalent amount, in a medium such as one in which the salt precipitates or in an aqueous medium followed by lyophilization.

The ligands which contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

The ligands which contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine and the like.

Non-limiting examples of the ligands for the inducible gene expression system utilizing the FK506 binding domain are FK506, Cyclosporin A, or Rapamycin. FK506, rapamycin, and their analogs are disclosed in U.S. Pat. Nos. 6,649,595 B2 and 6,187,757. See also U.S. Pat. Nos. 7,276,498 and 7,273,874.

The ligands described herein may be administered alone or as part of a pharmaceutical composition comprising an agrochemical acceptable carrier. In one embodiment, the pharmaceutical composition is in the form of a solution, a suspension, or a spray composition.

The term “ecdysone receptor-based,” with respect to a gene switch, refers to a gene switch comprising at least a functional part of a naturally occurring or synthetic ecdysone receptor ligand binding domain and which regulates gene expression in response to a ligand that binds to the ecdysone receptor ligand binding domain. Examples of ecdysone-responsive systems are described in U.S. Pat. Nos. 7,091,038 and 6,258,603.

### D. Regulatory Elements for the Polynucleotides or Vectors

Initiation control regions or promoters, which are useful to drive expression of a nucleic acid in the desired host cell are numerous and familiar to those skilled in the art. Virtually any promoter capable of driving these genes is suitable for the present invention including but not limited to: viral promoters, plant promoters, bacterial promoters, animal promoters, mammalian promoters, synthetic promoters, constitutive promoters, tissue specific promoter, developmental specific promoters, inducible promoters, light regulated promoters; CYC1, HIS3, GAL1, GAL4, GAL10, ADH1, PGK, PHO5, GAPDH, ADC1, TRP1, URA3, LEU2, ENO, TPI, alkaline phosphatase promoters (useful for expression in Saccharomyces); AOX1 promoter (useful for expression in Pichia); β-lactamase, lac, ara, tet, tryp, lPL, lPR, T7, tac, and trc promoters (useful for expression in Escherichia coli); and light regulated-, seed specific-, pollen specific-, ovary specific-, pathogenesis or disease related-, cauliflower mosaic virus 35S, CMV 35S minimal, cassava vein mosaic virus (CsVMV), APETALA1 (APi), LEAFY (LFY), Arabidopsis thaliana FLOWERING PROMOTING FACTOR 1 (AtFPF1), UNUSUAL FLORAL ORGANS (UFO), TERMINAL FLOWER 1 (TFL1), SHOOT MERISTEMLESS (STM), upstream region of the HISTONE H4 (H4A748), Arabidopsis thaliana TRANSLATIONALLY CONTROLLED TUMOR PROTEIN (AtTCTP), Cestrum yellow leaf curling virus (CmYLCV), Cotton leaf curl Gezira virus (C1CuGB-5), pepper huasteco yellow vein virus (PHYVV) and peanut chlorotic streak virus (PClSV), chlorophyll a/b binding protein, ribulose 1,5-bisphosphate carboxylase, shoot-specific, root specific, chitinase, stress inducible, rice tungro bacilliform virus, plant superpromoter, potato leucine aminopeptidase, nitrate reductase, mannopine synthase, nopaline synthase, ubiquitin, zein protein, and anthocyanin promoters (useful for expression in plant cells); animal and mammalian promoters known in the art include, but are not limited to, the SV40 early (SV40e) promoter region, the promoter contained in the 3′ long termnal repeat (LTR) of Rous sarcoma virus (RSV), the promoters of the ElA or major late promoter (MLP) genes of adenoviruses, the cytomegalovirus early promoter, the herpes simplex virus (HSV) thymidine kinase (TK) promoter, an elongation factor 1 alpha (EF1) promoter, a phosphoglycerate kinase (PGK) promoter, a ubiquitin (Ubc) promoter, an albumin promoter, the regulatory sequences of the mouse metallothionein-L promoter, and transcriptional control regions, the ubiquitous promoters (HPRT, vimentin, α-actin, tubulin and the like), the promoters of the intermediate filaments (desmin, neurofilaments, keratin, GFAP, and the like), the promoters of therapeutic genes (of the MDR, CFTR or factor VIII type, and the like), and promoters that exhibit tissue specificity and have been utilized in transgenic animals, such as the elastase I gene control region which is active in pancreatic acinar cells; insulin gene control region active in pancreatic beta cells, immunoglobulin gene control region active in lymphoid cells, mouse mammary tumor virus control region active in testicular, breast, lymphoid and mast cells; albumin gene, Apo AI and Apo AII control regions active in liver, alpha-fetoprotein gene control region active in liver, alpha 1-antitrypsin gene control region active in the liver, β-globin gene control region active in myeloid cells, myelin basic protein gene control region active in oligodendrocyte cells in the brain, myosin light chain-2 gene control region active in skeletal muscle, and gonadotropic releasing hormone gene control region active in the hypothalamus, pyruvate kinase promoter, villin promoter, promoter of the fatty acid binding intestinal protein, promoter of the smooth muscle cell α-actin, and the like. In some embodiments of the invention, the promoter is selected from the group consisting of a cauliflower mosaic virus 35S promoter, a cassava vein mosaic virus promoter, and a cauliflower mosaic virus 35S minimal promoter, an elongation factor 1 alpha (EF1) promoter, a phosphoglycerate kinase (PGK) promoter, a ubiquitin (Ubc) promoter, and an albumin promoter provided that the promoter selected is active in the plant into which it is introduced.

In addition, these expression sequences may be modified by addition of enhancer or regulatory sequences and the like. Enhancers that may be used in embodiments of the invention include but are not limited to: tobacco mosaic virus enhancer, cauliflower mosaic virus 35S enhancer, tobacco etch virus enhancer, ribulose 1,5-bisphosphate carboxylase enhancer, rice tungro bacilliform virus enhancer, and other plant and viral gene enhancers, and the like.

Termination control regions, i.e., terminator or polyadenylation sequences, may also be derived from various genes native to the preferred hosts. Optionally, a termination site may be unnecessary, however, in some embodiments, it is preferred if included. In some embodiments of the invention, the termination control region may be comprise or be derived from a synthetic sequence, synthetic polyadenylation signal, an SV40 late polyadenylation signal, an SV40 polyadenylation signal, a bovine growth hormone (BGH) polyadenylation signal, nopaline synthase (nos), cauliflower mosaic virus (CaMV), octopine synthase (ocs), Agrobacterium, viral, and plant terminator sequences, or the like.

The polynucleotide or vectors of the invention may also include a repression domain such as LxLxL from an AUX/IAA protein, such as those described in Tiwari, S. B. et al. (2004) Plant Cell 16(2):533-543. Examples include, but are not limited to, TELRLGLPG (SEQ ID NO: 37), TELRLGLPE (SEQ ID NO:38), TELCLGLPG (SEQ ID NO:39), TELTLGLPG (SEQ ID NO:40), TELTLALPG (SEQ ID NO:41), TDLRLGLSF (SEQ ID NO:42), TELDLALGL (SEQ ID NO:43), SELELGLGL (SEQ ID NO:44), MELDLGLSL (SEQ ID NO:45), IELGLTLSL (SEQ ID NO:46), IDLGLDLRT (SEQ ID NO:47), VNLSLSLTF (SEQ ID NO:48), KKLELKLGP (SEQ ID NO:49), KKLELRLHR (SEQ ID NO:50), and KRLELRLAP (SEQ ID NO:51). The repression domain may also be an ERF-associated amphiphilic repression (EAR) domain such as a SIERF36, or SIERF.F.1 (Accssion No. SGN-U564952) as described in Upadhyay, R. K. et al. (2014) PLOS One 9(7): e101995. Examples include, but are not limited to, NtERF3 (IDLDLNLAP) (SEQ ID NO:52), AtERF4 (LDLELNLPP) (SEQ ID NO:53), and AtSUPR (QDLDLELRL) (SEQ ID NO:54).

### E. Control of Flowering and Biomass

Controlled flowering addresses a major regulatory concern of potential gene flow from GE crops to closely related weeds. It enables development of beneficial traits (e.g. drought tolerance, resistance to pests and diseases, herbicide resistance) in plant species prone to outcrossing (e.g. in turfgrass). Controlled flowering also improves trait stewardship by controlling seed production, creates opportunity to achieve dramatic increase in yield and vigor through development of hybrid seeds in crops where it is not currently possible or economical; improves yield and quality of crops where natural switch to flowering is undesirable (bolting in vegetables (e.g. broccoli, lettuce, spinach), declined nutritional quality of forage crops (e.g. alfalfa)); and improves yield and decreases costs in crops where synchronized flowering/fruit setting is desirable (e.g. pineapples).

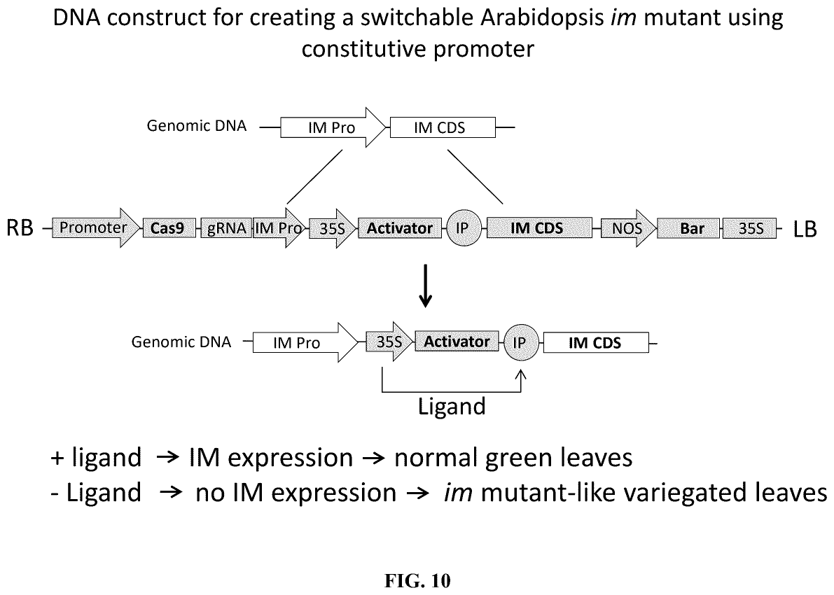

Flowering is controlled by various genes illustrated in FIG. 10. As used herein genes responsible for flowering may include, but are not limited to, any of these genes such as but not limited to the following (with gene identifiers in parentheses): CONSTANS (CO, AT5G15840), FLOWERING LOCUS D (FD, AT3G10390) or (At4g35900), SUPPRESSOR OF OVEREXPRESSION OF CO1 (SOC1, AT2G45660), TWIN SISTER OF FT (TSF, AT4G20370), LEAFY (LFY, AT5G61850), APETALA1 (APi, AT1G69120), SPL3 (At2g33810); SPL9 (At2g42200); SUC2 (At1g22710); FLC (At5g10140); β-TUBULIN-2 (At5g62690); FT (At1g65480); FUL (At5g60910); AGL42 (At5g62165); TFL1 (At5g03840); FloweringMIR156a (At2g25095).

### F. Controlled Resistance to Disease

Another use of the gene switch systems of the invention is the controlled delivery of compounds for pest and disease protection whereby a combination of novel pest and/or disease control chemistries are expressed only after application of ligand spray or activator substance. By way of illustration, but not limitation, multiple plant genes have been identified that have direct anti-microbial activity or act as positive regulators of the innate immune system; constitutive expression of these genes typically results in a loss of fitness for the plant. Precision control of defense related gene expression through switch ligand-dependent transcription activation enables transgenic disease resistance while reducing the metabolic or fitness cost associated with producing a transgene constitutively. In some embodiments, it is desireable to limit the expression of these antimicrobial compounds or positive regulators to key time points with the highest disease pressure. This can not only reduce the stress of constitutive expression of a transgene, but can also reduce the accumulation of novel chemistries in the environment. Moreover, expressing multiple chemistries with distinct modes of action and limiting their accumulation in the environment, reduces the long-term risk of resistant populations of insects or diseases developing in the field.

Examples of antimicrobial peptides that can be expressed in embodiments of the invention include defensins. Defensins are small, stable cysteine-rich peptides produced by plants that form part of the plant's innate immune system. Defensins are known to have antifungal activity and have also been shown to have activity against insects and some have antimicrobial activity (Stotz, H. U. et al. (2009) Plant Signal Behav. 4(11):1010-1012). For example, it has been demonstrated that wasabi defensin (WI1) imparted increased resistance to Magnaporthe grisea, Erwinia carotovora and Botrytis cinerea in rice, potato and orchid, while chili defensin (cdefl) has been shown to impart resistance to Phytophthora infestans and Fusarium sp. in tomatoes (Stotz et al. (2009)). Expression of dahlia defensin (Dm-AMP1) has been demonstrated to impart resistance to Magnaporthe oryzae and Rhizoctonia solani in rice and Phytophthora palmivora in papaya (Stotz et al. (2009)).

Examples of antifungal defensins that may be used in the invention include, but are not limited to Defensins, such as Ah-AMP1, AX1, AX2, At-AFP1 (LCR67), AFP2B, Psdl, Psd2, J1-1, J1-2, Tk-AMP-D1, Tk-AMP-D1.1, Tk-AMP-D2, Tk-AMP-D3, Tk-AMP-D4, Tk-AMP-D5, Tk-AMP-D6, Tk-AMP-D6.1, Tm-AMP-D1.2, PhD1, PhD2, NaD1, MsDefl, MtDef2, MtDef4, MtDef5, Rs-AFP1, Rs-AFP2, Sa-AFP2 and VrD1, which have activity against Alternaria, Botrytis, Fusarium, Pyricularia, Verticillium, Aspergillus, Saccharomyces, Trichophyton, Cercospora, Cladosporium, Leptosphaeria, Penicillium, Trichoderma, and Septoria; Snakins, such as Snakin-1 (StSN1), Snakin-2 (StSN2), which have activity against Botrytis (e.g., Botrytis cinerea), Fusarium, Plectosphaerella, Colletotrichum, Bipolaris, and Aspergillus; Heveins, such as AC-AMP1, AC-AMP2, Ar-AMP, EAFP1, EAFP2, Ee-CBP, Fa-AMP1, Fa-AMP2, IWF4, PN-AMP1, PN-AMP2, and WjAMP1 which have activity against Alternaria, Ascochyta, Botrytis (e.g., Botrytis cinerea), Colletotrichum, Fusarium, Trichoderma, Verticillium, Phytophthora, Gibberella, Mycosphaerella, Neurospora, Phoma, Pythium, Rhizoctonia, Helinthosporium, Geotrichum, and Saccharomyces; Thionins, such as Alpha-1-Purothionin, Alpha-2-Purothionin, Alpha-hordothionin, β-hordothjionin, BTH6, Pp-AMP1, Pp-AMP2, Tu-AMP-1, Tu-AMP2, VtA3, and VtB, which have activity against Fusarium, Sclerotinia, Phytophtora, and Geotrichum; Lipid Transfer Proteins such as La-LTP (LJAFP), Ace-AMP1, Hy-LTP Cw-18 (PKG2316), Hy-LTP4.1 (LTP4.1)(CW21), IWF1 (Bv-LTP1), IWF2 (Bv-LTP2), and Pa-LTP1, which have activity against Fusarim, Pythium, Sclerotium, Alternaria, Ascockyta, Aspergillus, Bipolaris, Botrytis (e.g., Botrytis cinerea), Cerospora, Colletotrichum, Penicillium, Pyricularia, Rhizoctonia, Saccharomyces, Sclerotinia, Trichoderma, Verticillium, Clavibacter, Pyrenopkora, and Nectria; Cyclotides, such as Circulin-A, Circulin-B, Cyclopsychotride-A, Kalata-B1, which are active against Candida; Shepherins, such as Shepherin I and Shepherin II, which are active against Candida, Cryptococcus, Saccharomyces, Alternaria, Aspergillus, and Fusarium; MBP-1 family peptides, which are active against Alternaria Fusarium, Sclerotinia, and Aspergillus; Vicilin-like peptides such as MiAMP2, MiAMP2b, MiAMP2c-1, MiAMP2c-2, MiAMP2c-3, and MiAMP2d, which are active against Alternaria, Ceratocystis, Cercospora, Chalara, Fusarium, Leptosphaeria, Sclerotinia, Verticillium, Saccharomyces, and Phytophthora; Impatiens family peptides such as Ib-AMP1, Ib-AMP2, Ib-AMP3, and Ib-AMP4, which have activity against Alternaria, Botrytis (e.g., Botrytis cinerea), Cladosporium, Fusarium, Penicillium, Trichoderma, and Verticillium; 0-Barrelin, such as MiAMP1 which has activity against Saccharomyces; knottins family peptides, such as MJ-AMP1, MJ-AMP2, Mc-AMP1, and Pa-AMP1 (PAFP-S), which have activity against Alternaria, Ascochyta, Botrytis (e.g., Botrytis cinerea), Cercospora, Colletotrichum, Fusarium, Nectria, Phoma, Pyrenophora, Pyricularia, Rhizoctonia, Verticiliium, and Venturia.

Some defensins have antimicrobial activity. Examples of defensins family peptides with antimicrobial activity that may be used in the invention include, but are not limited to Defensins, such as Ah-AMP1, AX1, AX2, Cp-thionin-2, Fabatin-1, Fabatin-2, and VrD1, which have activity against Gram-positive bacteria (e.g., Bacillus, Enterococcus, and Staphylococcus); Snakins, such as Snakin-1 (StSN1), Snakin-2 (StSN2), and ZmGASA-like, which have activity against Gram-positive bacteria (e.g., Clavibacter, Ralstonia, Listeria, and Rhizobium) Erwinia (e.g., Erwinia amylovora), Escherichia, and Pseudomonas; Heveins, such as AC-AMP1, AC-AMP2, Fa-AMP1, Fa-AMP2, PN-AMP1, PN-AMP2, and WjAMP1 which have activity against Gram positive bacteria (e.g., Bacillus, Clavibacter) and Escherichia; Thionins, such as Pp-AMP1, Pp-AMP2, Tu-AMP-1, Tu-AMP2, which have activity against Erwinia, Agrobacterium, Clavibacter and Curtobacterium; Lipid Transfer Proteins such as La-LTP, Ace-AMP1, Lc-LTP1, Lc-LTP2, Lc-LTP3, Lc-LTP4, Lc-LTP5, Lc-LTP6, Lc-LTP7, Lc-LTP8, Pa-LTPlwhich have activity against Gram positive bacteria (e.g., Bacillus, Staphylococcus, Sarcina), Pseudomonas, Ralstonia, Agrobacterium, and Escherichia; Cyclotides, such as Circulin-A, Circulin-B, Cyclopsychotride-A, Kalata-B1, which are active against Gram positive bacteria (e.g., Staphylococcus, Micrococcus), Pseudomonas, Proteus, Klebsiella, and Escherichia; Shepherins, such as Shepherin I and Shepherin II, which are active against Gram positive bacteria (e.g., Bacillus, Staphylococcus, Streptococcus), Erwinia, Escherichia, Pseudomonas, Salmonella, and Serratia; MBP-1 family peptides, which are active against Gram positive bacteria (e.g., Clavibacter), and Escherichia; Vicilin-like peptides such as MiAMP2, MiAMP2b, MiAMP2c-1, MiAMP2c-2, MiAMP2c-3, and MiAMP2d, which are active against Gram positive bacteria (e.g., Clavibacter); Impatiens family peptides such as Ib-AMP1, and Ib-AMP4, which have activity against Gram positive bacteria (e.g., Bacillus, Micrococcus, Staphylococcus, Streptococcus) Xanthomonas, Erwinia, Escherichia, Proteus, and Pseudomonas; knottins family peptides, such as MJ-AMP1, MJ-AMP2, Mc-AMP1, and Pa-AMP1 (PAFP-S), which have activity against Gram positive bacteria (e.g., Bacillus, Sarcina, and Staphylococcus).

Some defensins have activity against insects. These include, for example, but not by way of limitation, defensins such as VrD1, which has activity against Spodoptera frugiperda; Cyclotides such as Circulin-B, Kalata-B1, and Kalata-B2, which have activity against Helicoverpa.

### G. Control of Other Traits

Controlled gene expression using the polynucleotides or vectors of the invention may be applied to any trait of interest. Various phenotypic traits include stress resistance (e.g., drought and herbicides), tunable traits such as type and intensity of color and aroma in flowers, and controlled accumulation of specific nutrients in fruits and vegetables such as differentiating taste and nutritional value to satisfy different groups of consumers. Timing of expression can be important such that one may select to turn on genes at the proper time in the plant's life, and even the production of high value compounds (exogenous active pharmaceutical ingredients, biopolymers, etc.). In some embodiments it is necessary to control the timing of compound accumulation (switching it on right before harvesting) because of the fitness cost. In other embodiments, the control of expression is timed in the life of the plant and in various plant tissues using tissue-specific prototers to express the gene of interest at specific stages of the plant (e.g., flowering, seed production) in order to achieve the goal desired by the grower.

### H. Uses of Controlled Traits

Uses of the control of flowering include, but are not limited to, improvement in crop yield and quality in conjunction with lower cost profile for select fruits and vegetables; increased biomass production in forage crops by prolonging vegetative state; on-demand resistance for environmental and biological stressors such as drought, pests and disease; development of beneficial traits in plant species prone to outcrossing such as turfgrass; enhanced plant production of high-value compounds including active pharmaceutical ingredients and biopolymers; approach to reduce potential environmental impact from GMO crops addressing significant global concerns; regulated accumulation of specific nutrients in fruits and vegetables providing an ability to adjust the taste or nutritional value to satisfy market demands; tune traits such as color and aroma in flowers, as well as provide florists ability to have flowering occur onsite lowering lost productivity commonplace in the industry; and more efficient and effective commercial seed production of plants.

Examples of these crops that may be controlled using the compositions and methods of the invention include, but are not limited to, alfalfa sprouts, apples, apricots, artichokes, Asian pears, asparagus, atemoyas, avocados, bamboo shoots, bananas, beans, bean sprouts, beets, belgian endive, bitter melons, bell peppers, blackberries, blueberries, bok choy, boniato, boysenberries, broccoflowers, broccoli, broccolini, brussels sprouts, butter lettuce, cabbage, cantaloupe, carambola, carrots, casaba melons, cauliflower, celery, chayotes, cherimoyas, cherries, coconuts, coffee, collard greens, corn, cotton, cranberries, cucumbers, dates, eggplant, endive, escarole, feijoa, fennel, figs, garlic, gooseberries, grapefruit, grapes, green beans, green onions, collard greens, mustard greens, guava, hominy, honeydew melons, horned melons, iceberg lettuce, Jerusalem artichokes, jincama, kale, kiwifruit, kohlrabi, kumquats, leeks, lemons, lettuce, lima beans, limes, longan, loquats, lychees, madarins, malangas, mandarin oranges, mangos, marijuana, mulberries, mushrooms, napas, nectarines, okra, onions, oranges, papayas, parsnip, passion fruits, paw-paws, peaches, peanut, pears, sugar snap peas, green peas, peppers, persimmons, pineapples, plantains, plums, pomegranates, potatoes, prickly pears, pummelos, pumpkins, quince, radicchio, radishes, raspberries, red cabbage, rhubarb, romaine lettuce, rutabaga, shallots, snow peas, soybeans, spinach, sprouts, squash, strawberries, string beans, sweet potatoes, tangelo, tangerines, tomatillo, tomatoes, turnip, ugli fruit, watermelons, water chestnuts, watercress, waxed beans, yams, yellow squash, yuca/cassava, and zucchini squash.

Examples of flowers that may be controlled using the compositions and mthods of the invention include, but are not limited to African daisy, Agapanthus, Ageratum houstonianum, Alchemilla, Allium, Alyssum, Amaranthus, Amaryllis, Anemone, Angelonia, Anthurium, Artemisia, Asclepias syriaca, Aster, Astilbe, Astrantia, Aubreita deltoidea, baby's breath, bachelor button, balloon flower, bee balm, begonia, bellflower, blanketflower, Bergenia, black-eyed Susan, blanket flower, blazing star, bleeding heart, bluebell, blue-eyed grass, blue star flower, Bouvardia, Bougainvillea, broom, Buddleja, bush morning glory, buttercup, butterfly weed, butterfly bush, Calendula, California poppy, calla lily, Calliandra, Camellia, Campenula, candytuft, canna lily, cape primrose, cardinal flower, carnation, catmint, celosia, chrysanthemum, Clarkia, clover, clematis, cockscomb, columbine, coneflower, coral bells, Coreopsis, Cosmos, Cotoneaster, Crocus, creeping phlox, Crocosmia, crown imperial, cuckoo flower, Cyclamen, Dahlia, day lily, Delphinium, Echium, English bluebell, Erigeron, evening primrose, Euphorbia, flannel flower, flax flower, floss flower, forget-me-not, Forsythia, foxglove, frangipani, freesia, fuschia, gardenia, geranium, gas plant, Gaura, gayfeather, Gerbera, Gladiolus, globeflower, goldenrod, grape hyacinth, Gypsophila, heather, Hebe, Helenium, Heliotrope, Hellebore, hibiscus, hollyhock, honeysuckle, hosta, hyacinth, hydrangea, Hypericum, hardy geranium, hybrid tea roses, Iceland poppy, ice plant, Ilex, Impatiens, Ipheion uniflorum, iris, Ixia, Ixora, Jaborosa, Jacob's ladder, Jamesia americana, jasmine, Jupiter's beard, kaffir lily, Kalmia, kangaroo paw, Kerria, Knautia macedonica, Kniphofia, Kolkwitzia, lady's slipper, Lamium, Lantana, larkspur, Lavatera, lavender, Lechenaultia, lilac, lily, lily of the valley, Linaria, lisianthus, lobelia, loosestrife, lotus, lunaria, lupin, magnolia, Maltese cross, Mandevilla, Marguerite daisy, marigold, Matthiola, mayflower, Meconopsis, mimosa, Mina lobate, mock orange, monk's hood, moonflower, morning glory, Muscari, narcissus, nasturtiums, Nemesia, Nemophila, Nerine, New Guinea impatien, Nicotiana, Nierembergia, Nigella, Nolana, oleander, orchid, oriental lily, oriental poppy, Osteospermum, oyster plant, ox eye daisy, painted daisy, pansy, passion flower, peace lily, Pelargonium, Penstemon, peony, Persian buttercup, Peruvian lily, petunia, pincushion flower, pink lady's slipper, pointsettia, Polyanthus, poppy anemone, Portulaca grandiflora, Primula, Quaker ladies, Queen Anne's lace, Queen's cup, Queen of the meadow, quince, rain lily, Ranunculus, Rhododendron, rock rose, Rondeletia, rose, rose of Sharon, Salvia splendens, Saponaria, Scabiosa, Scaevola, scented geranium, Scilla, Sedum, shasta daisy, shrub roses, Silene, silver lace vine, snapdragon, snowball bush, snowdrop, snowflake, statice, strawflower, sun drop, sunflower, sweet pea, Syringa, tea rose, tiger flower, tiger lily, Tithonia, Trillium, Triteleia, Tritonia crocata, trumpet vine, tuberose, tulip, urn plant, Ursinia, Uva ursi, Verbena, Veronica incana, Vinca, Viola tri-colour, Violet, Virginia creeper, wallflower, wandflower, water lily, Watsonia, wax plant, Wedelia, Weigela, wild rose, wild violet, winter aconite, winterberry, winter jasmine, wishbone flower, wisteria, wooly violet, Xerophyllum, Xylobium, Xylosma, yarrow, yellow angel, yellow bell, yellow-eyed grass, yellowhorn, Zenobia, and zinnia.

Examples of grains that may be controlled using the compositions and methods of the invention include, but are not limited to barley, buckwheat, bulgur wheat, corn, durum wheat, einkorn, emmer, farro, fonio, kamut, millet, oats, rice, rye, semolina wheat, sorghum, spelt, teff, triticale, wheat, bamboo shoots, barleygrass, lemongrass, molasses, rapadura, sugarcane, wheatgrass, Amaranth, Coxcomb, pitseed goosefoot, quinoa, chia, acacia seed, and wattleseed.

Examples of turf grass that may be controlled using the compositions and mthods of the invention include, but are not limited to Kentucky bluegrass, perennial ryegrass, tall fescue, fine fescue, creeping bentgrass, creeping red fescue, hard fescue, chewings fescue, Bermudagrass, buffalograss, kikuyugrass, St. Augustine, and zoysia.

Examples of forage crops that may be controlled using the compositions and methods of the invention include but are not limited to alfalfa, alsike clover, annual lespedeza, birdsfoot trefoil, crownvetch, ladino clover, red clover, Sericea lespedeza, sweetclover, white Dutch clover, big bluestem, caucasian bluestem, Indiangrass, little bluestem, pearl millet, side-oats grama, switchgrass, and triticale.

In certain preferred embodiments, the plants are potato, sugar beet, alfalfa, silage corn hay, pear apple, pineapple, orange, grapefruit, coffee, soybeans, snapbeans, kidney beans, zinnias, turfgrass, sorghum, rice, wheat, barley, green bean, red bean, potato, oat and millet; Arabidopsis, Chinese cabbage, radish, red pepper, strawberry, tomato, watermelon, cucumber, cabbage, melon, squash, stone-leek, onion, ginseng, tobacco, marijuana, cotton, sesame, sugar cane, sugar beet, wild sesame, peanut, rape, peach, date, western Actinidia, grape, persimmon, plum, apricot, banana, ryegrass, red clover, orchard grass and tall fescue, maize, miscanthus, and switchgrass.

In some embodiments, flower color may be controlled on demand using the gene switch strategies of the invention. For example, a gene responsible for flower color such as chalcone synthase A of petunias which is responsible for purple color can be influenced using a gene switch system in which a dsRNA is expressed in the petunia when provided with ligand. The dsRNA has the effect of inhibiting chalcone synthase A and the flowers will not be deep purple, instead, the flowers will have low level of purple color with increased amounts of the dsRNA. At high levels, the flowers will be white. Similarly, other genes responsible for flower color, aroma, and other traits can be targeted and operatively linked to the gene switch system of the invention for on-demand expression or inhibition of a trait.

### I. Methods of Making the Polynucleotides or Vectors

Various non-limiting arrangements and types of the vector elements are illustrated in the Examples Section and in the Figures, but other types of promoters, activators, terminators, etc., and arrangements of the elements (5′ or 3′ of one another) can be made to achieve desired outcomes as will be apparent to one of skill in the art.

The polynucleotide vectors of the invention may be made by methods that are well-known in the art. DNA construct may also be made such that elements for gene editing may be containing both an EcR-based gene switch and gene editing elements such as zinc-finger nucleases, TALENs or CRISPR elements. For example, but not by way of limitation, CRISPR elements (Cas9 and gRNA elements) may be incorporated into a vector such that the vector targets a locus in the genome of the plant and the CRISPR-Cas9 and guide RNA elements direct transformation of the plant through homology directed recombination (HDR) to insert the gene switch between the gene of interest promoter and its coding sequence to create an insertional mutation of gene of interest to create a gene under the control of a gene switch of the invention, while the other components of the DNA construct including the CRISPR-Cas9 and gRNA elements are not integrated at the locus. Application of the activating ligand turns the gene back on to restore normal function.

In other methods, homologous recombination may be used to replace the endogenous gene of interest (e.g., a gene responsible for flowering) or an exogenous gene to be controlled by a gene switch into a predetermined locus in the plant. In this way, the endogenous plant gene will be controllable by application of the chemical ligand “on-demand.”

In some embodiments of the invention, control of a plant gene, (e.g., for flowering) may be accomplished by introducing Bacillus amyloliquefaciens Barnase into the plant. Barnase (“BActerial RiboNucleASE”) is a nuclease that has been shown to inhibit gene expression as it degrades nucleic acid and prevents expression in plants. Barnase may be expressed in the method of the invention by using tissue-specific promoters to limit Barnase expression to a particular tissue to inhibit the targeted gene of interest. Generalized expression of Barnase could be lethal to the plant. As an example, one could target a gene for flowering using a flowering-specific promoter to limit the expression of Barnase to flower tissue. Barnase may be operatively linked to a flower-specific promoter (e.g., LFY, AP1, H4A, etc.). Floral-meristem controlled expression of Barnase should prevent flowering in the plant. To control flowering, an EcR-based gene switch of the invention may be introduced with Bacillus amyloliquefaciens Barstar which is operatively linked to a regulatory element 5′ of of Barstar wherein said EcR-based gene switch binds said regulatory element in the presence of the activating chemical ligand. Barstar is an inhibitor of Barnase and controlled expression of the Barstar inhibitor allows the plant to flower. Introduction of Barnase under the control of a plant tissue-specific promoter may be on the same polynucleotide or vector of the invention or on a separate polynucleotide or vector.

Other ways of accomplishing control include providing a vector or polynucleotide to direct expression of an inhibitor of an endogenous gene of interest constitutively such that the trait is turned off in the plant. The gene switch of the invention may be introduced with a gene to override the repressor of expression. By way of example and not by way of limitation, a plant may be made to constitutively express miR156 to inhibit SPL and thereby inhibit flowering in the plant. The plant may also be transformed with a gene switch of the invention to control a mutant form of SPL that has altered codon usage so the miR156 molecule cannot hybridize with it to prevent expression. Application of ligand leads to expression of the SPL (same amino acid sequence, but altered mRNA sequence) and the SPL protein produced therefrom leads to flowering in the plant.

Vectors may be introduced into the desired host cells by methods known in the art, e.g., Agrobacterium-mediated transformation, transfection, electroporation, microinjection, transduction, cell fusion, DEAE dextran, the flower dipping method, use of a gene gun (biolistics) and the like. The EcR-based gene switch may be introduced into the plant separately from the gene to be controlled and inserted to control the native gene of interest. Alternatively, the native gene to be controlled may be replaced with the gene switch and control elements on the polynucleotide or vector of the invention.

It will be understood that the above description is intended to be illustrative of the invention and is not limiting. Those of skill in the art may make various modifications without departing from the spirit of the invention, which is defined by the appended claims.

### EXAMPLES

### Example 1

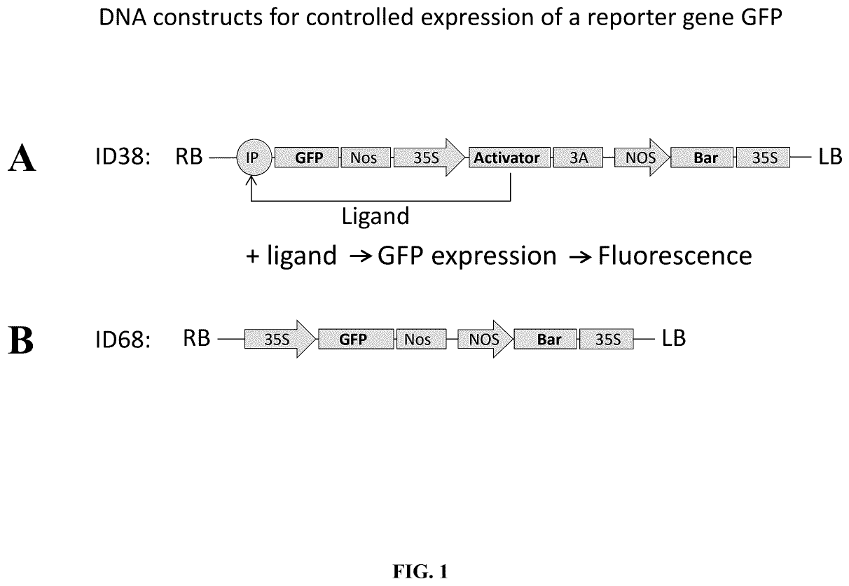

Transform wild type Arabidopsis to introduce inducible GFP reporter: DNA construct ID38, which is shown diagrammatically in FIG. 1A, may be used to transform wild type (wt) Arabidopsis. The construct contains both the inducible promoter (IP) composed of 5 GAL4 regulatory elements (GAL4-RE) placed upstream of the minimal CaMV-35S promoter and the multidomain activator (AP—activator protein or Activator) gene (VGE(E68V/V184I/Y204E) under the control of CaMV-35S strong constitutive promoter. In addition, the construct contains the Nos:Bar gene to allow selection of transgenic plants with glufosinate-ammonium that is the active ingredient of herbicide formulations sold under different brand names including LIBERTY® 280SL and BASTA@herbicides.

GFP gene will not be expressed until methoxyfenozide (activator ligand—AL) is provided to mobilize the activator proteins for binding to the GAL4-RE and switching on the IP. Methoxyfenozide is the active ingredient of INTREPID® 2F commercial insecticide. Since GFP expression can be conveniently monitored in live plants under a fluorescence microscope, the transgenic Arabidopsis can be used to study the dosage, timing, and half life time of the ligand application. The DNA construct ID68, shown diagrammatically in FIG. 1B, containing GFP under control of strong constitutive promoter CaMV-35S may be used to transform wt Arabidopsis to produce positive control plants that will express GFP constitutively.

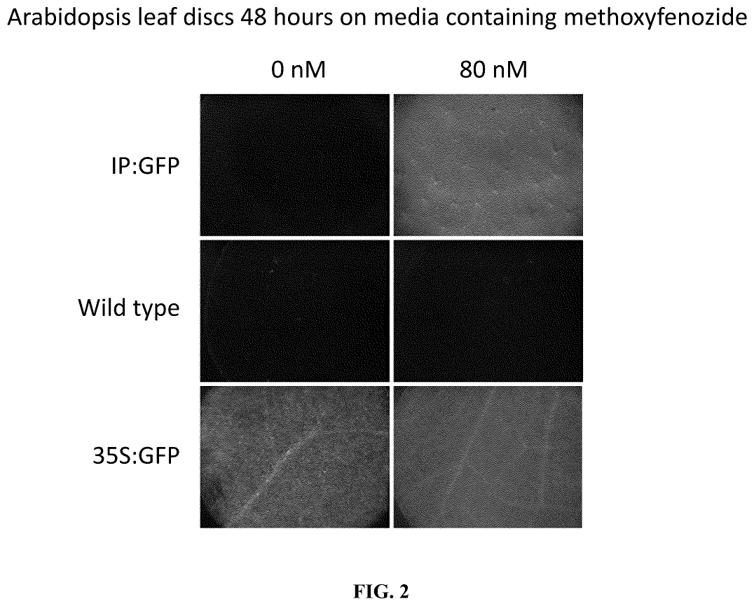

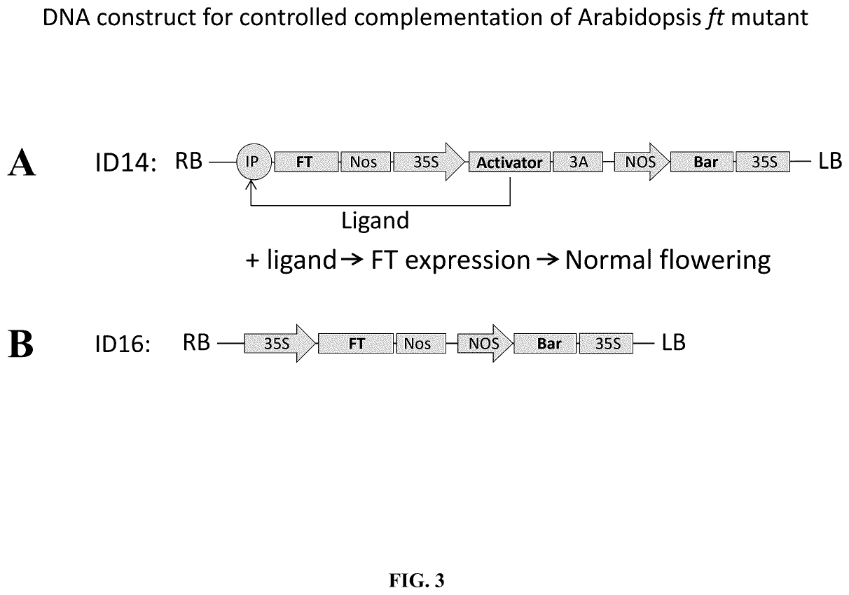

Briefly, the DNA constructs shown in FIG. 1 were built following standard molecular cloning procedures and transformed into disarmed Agrobacterium tumefaciens strain AGL1. Wild type (wt) Arabidopsis plants were then transformed separately with the Agrobacterium strains carrying constructs ID38 and ID68 by flower dipping method. T1 seeds were harvested and germinated in soil. 5 day old T1 seedlings were sprayed with LIBERTY® 280SL herbicide to kill off non-transgenic plants. Selected transgenic plants were analyzed by quantitative PCR (qPCR) and PCR to check transgene copy numbers and transgene intactness. Specific primers for Bar gene (Bar-F1/Bar-R1/Bar-T1) and endogenous PDS gene as control (AtPDS-F1/AtPDS-R1/AtPDS-T1) were used (Primers used in the PCR assays are shown in Table 1). Events with single copy of the transgenes were grown in growth chambers under 16 hours long day photoperiod to evaluate phenotypes. Plants transformed with the positive control DNA construct 35S:GFP (ID68) showed strong GFP expression in leaves. No GFP expression was observed in plants transformed with the DNA construct IP:GFP (ID38). Leaf samples were collected from the plants and incubated on media containing the ligand methoxyfenozide at different concentrations to induce gene expression. GFP expression was induced with as low as 16 nM methoxyfenozide in 24 hr in the transgenic plants samples with IP:GFP but not in the wt control as shown in FIG. 2. Plants transformed with the 35S:GFP construct expressed GFP constitutively either with or without ligand induction.

### Example 2

### Transform Arabidopsis Mutants to Achieve Inducible Restoration of Wild-Type Phenotype:

### A. FT Mutant

DNA construct ID14, shown diagrammatically in FIG. 3A, is similar to that described in Example 1, except that the GFP is replaced with a wt FLOWERING LOCUS T (FT, AT1G65480) gene and may be used to transform a homozygous ft late flowering Arabidopsis mutant. Transgenic FT gene may similarly be turned on by applying methoxyfenozide ligand to give a dominant phenotype restoring late flowering to normal flowering. The advantage of using the late flowering ft mutant is to simplify phenotype analysis. The flowering time of transgenic plants can be compared to wt and ft plants grown side by side under the same long day or short day condition with or without applying methoxyfenozide ligand. DNA construct ID16, shown diagrammatically in FIG. 3B, containing FT under the direct control of CaMV-35S promoter may be used to transform ft mutant Arabidopsis to produce positive control plants that will express FT constitutively. Overexpression of FT under a strong constitutive promoter such as 35S will induce premature early flowering.

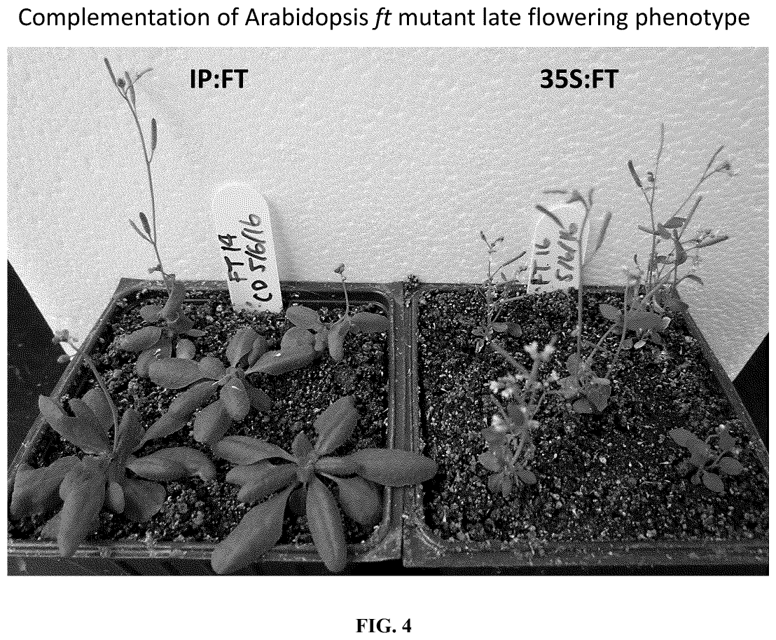

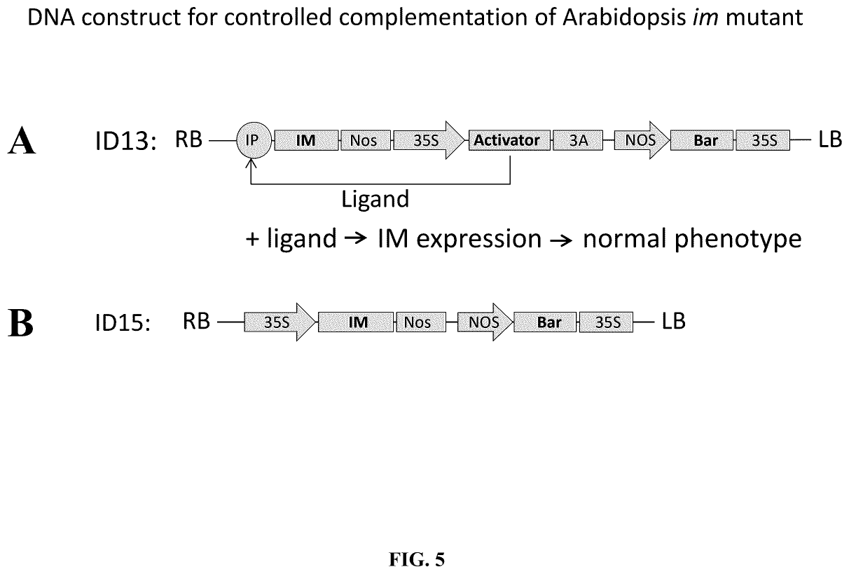

Briefly, the DNA constructs shown in FIG. 3 were transformed into homozygous ft mutant Arabidopsis by flower dipping method. T1 seeds were harvested and germinated in soil. 5 days old T1 seedlings were sprayed with LIBERTY® 280SL herbecide to kill off non-transgenic plants. Selected transgenic plants were analyzed by Bar-specific quantitative PCR (qPCR) to check transgene copy number. Events with low copy number of the transgenes were grown in growth chambers to evaluate phenotypes. In the absence of ligand methoxyfenozide some of the transgenic plants with IP:FT construct (ID14) showed the expected late flowering behavior just like the ft mutant and null segregants, while others showed early flowering, suggesting that the IP:FT cassette in these plants might be able to express enough FT to induce early flowering even in the absence of ligand induction. Plants transformed with the ID16 construct overexpressed FT constitutively that resulted in extremely early flowering and stunted phenotype as shown in FIG. 4. Non-flowering IP:FT plants were selected and sprayed with the ligand methoxyfenozide which induced flowering and allowed for normal seed set. Select T1 plants were grown to maturity and seeds were used to generate T2 and T3 progeny plants. Further detailed evaluation of the transgenic lines were done in T2 and T3 generation plants (as described in Example 7).

### B. Im Mutant.

DNA construct ID13, shown diagrammatically in FIG. 5A, is similar to that described in Example 1 except that the GFP is replaced with a wt IMMUTANS (IM) gene (AT4G22260). This construct may be used to transform Arabidopsis homozygous immutans variegation mutant (im) CS3639 as obtained from Aridopsis Biological Resource Center (ABRC). Transgenic wt IM gene may similarly be turned on by applying methoxyfenozide ligand to give a dominant phenotype where the white-green variegated leaves are restored to normal green leaves. The advantage of using the non-lethal variegation im mutant is to simplify phenotype analysis by the visual observation of leaf colors.

Alternatively, a recessive lethal albino T-DNA insertion mutant of the phytoene desaturase gene (PDS, locus AT4G14210) can be restored by expressing the wt PDS under the EcR-based gene switch control.

DNA construct ID15, shown diagrammatically in FIG. 5B, containing wt IM under direct control of CaMV-35S promoter may be used to transform im mutant Arabidopsis to produce positive control plants that will express IM constitutively.

Briefly, the DNA construct shown in FIG. 5A may be transformed into homozygous im Arabidopsis by flower dipping method. T1 seeds are then harvested and germinated in soil. 5 days old T1 seedlings are sprayed with LIBERTY® 280SL herbicide to kill off non-transgenic plants. Selected transgenic plants are analyzed by quantitative PCR (qPCR) and PCR to check transgene copy numbers and transgene intactness. Events with single intact copy of the transgenes are grown in growth chambers to observe phenotypes. In the absence of methoxyfenozide ligand the transgenic plants with IP:IM construct should have variegated leaves just like the im mutant plants. Selected plants may then be sprayed with the ligand methoxyfenozide and newly grown leaves should no longer be variegated due to EcR-based gene switch system driving expression of IM. Plants transformed with the construct in FIG. 5B would overexpress IM constitutively and would produce normal green leaves.

### Example 3

Targeting genes in plants by inserting EcR-based gene switch to control a phenotypic trait. One could also take advantage of the recent CRISPR gene targeting technology (Li et al. (2015) Plant Physiol. 169:960-970) to introduce inducibility to a plant gene. For example, a flowering control gene such as the FLOWERING LOCUS T (FT) florigen gene may be targeted by inserting EcR-based gene switch system between the FT promoter and coding sequence of the FT gene. The interruption of the key flowering gene FT will likely create a late flowering mutant that can be restored by turning the same FT gene expression back on by the EcR-based gene switch when appropriate ligand is applied. The native FT promoter is appointed to express the activator (AP) to mimic the natural expression profile of the FT gene and minimize potential pleiotropic effects from the overexpression of FT if using a strong constitutive promoter such as 355.

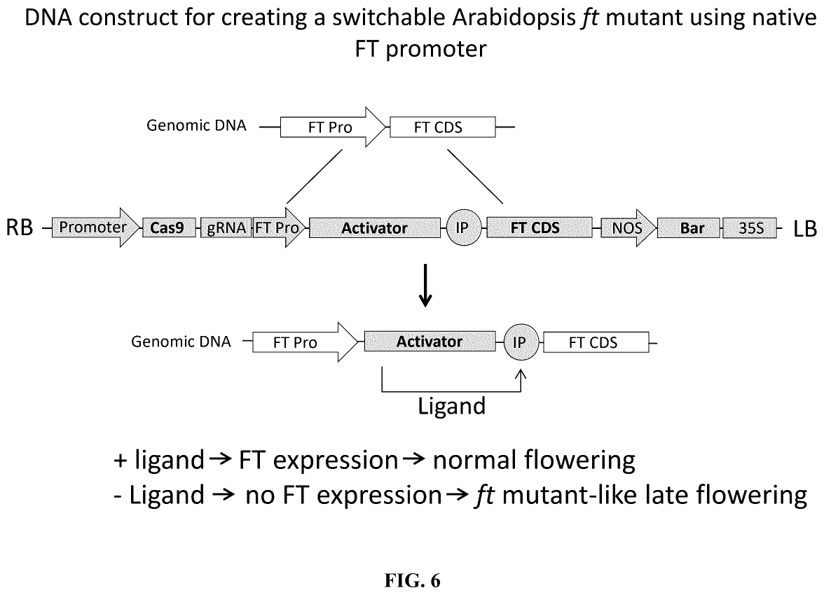

A DNA construct, such as shown diagrammatically in FIG. 6, containing both an EcR-based gene switch and CRISPR elements (Cas9+gRNA) targeting the FLOWERING LOCUS T (AT1G65480) gene is transformed into wt Arabidopsis. CRISPR mediated gene targeting through homology directed recombination (HDR) places the Activator+IP elements between the FT promoter and its coding sequence to create an insertional mutation of FT gene that leads to a late flowering phenotype. Other components of the DNA construct including the Promoter:Cas9+gRNA and Nos:Bar expression cassettes will not be integrated at the FT locus. Application of the methoxyfenozide ligand turns the FT gene back on and restores the late flowering FT mutation to normal flowering.

Briefly, a DNA construct as illustrated in FIG. 6 (middle) is transformed into wt Arabidopsis by flower dipping method. T1 seeds are harvested and germinated in soil. 5 days old T1 seedlings are sprayed with LIBERTY® 280SL herbicide to ensure than only transgenic plants survive. Transformants are analyzed by qPCR to check transgene copy number. Events with single copy of the transgenes are grown in growth chambers to evaluate genotypes. FT locus is further analyzed by PCR to check the targeted FT for gene interruption, and the targeted insertion site is sequenced to confirm that the insertion does not change the FT coding sequence. Events with an HDR insertion of the transgene or indel at the FT target site are grown in growth chambers to observe phenotypes. The majority of transgenic events with randomly integrated transgenes are discarded. Since ft is a recessive mutation, no late flowering phenotype occurs in hemizygous T1 generation.

T2 seeds are germinated into plants and homozygous transgenic plants identified by qPCR are then grown in growth chambers to evaluate for late flowering phenotype. Some of the plants are sprayed with methoxyfenozide and some are not (controls). The flowering time of sprayed and untreated plants are compared to wt andft plants grown side by side under the same conditions. Typically, Arabidopsis plants flower in ˜4 weeks of initiation in soil under long day (16 hours) photoperiod. Plants showing a delay or absence of flowering in the absence of methoxyfenozide ligand and normal flowering in the presence of the ligand should be insertional mutants and may be selected for further analysis of EcR switch controlled flowering and FT gene expression level.

### Example 4

### Targeting Genes in Plants by Inserting an Enhanced EcR-Based Gene Switch to Control a Phenotypic Trait.

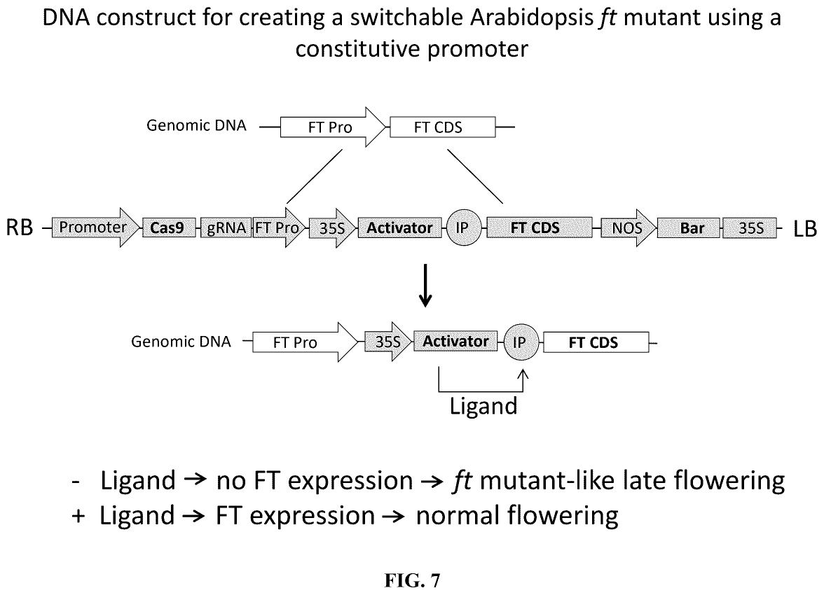

In another embodiment using CRISPR gene targeting technology, a DNA construct shown diagrammatically in FIG. 7, containing both an EcR-based gene switch using a strong 35S promoter and CRISPR elements (Cas9+gRNA) targeting the FLOWERING LOCUS T (AT1G65480) gene is transformed into wt Arabidopsis. The only difference between the two DNA constructs in FIG. 6 and FIG. 7 is that the latter has the 35S promoter expressing the Activator protein. CRISPR mediated gene targeting through homology directed recombination (HDR) places the 35S:Activator+IP elements between the FT promoter and its coding sequence to create an insertional mutation of FT gene that leads to a late flowering phenotype. Other components of the DNA construct including the Promoter:Cas9+gRNA and Nos:Bar expression cassettes will not be integrated at the FT locus. The 35S promoter is appointed to express more EcR-based gene switch proteins to trigger the switch for overexpression of the FT gene. Application of the methoxyfenozide ligand turns the FT gene on at higher level than the wt to convert theft late flowering mutation to early flowering.

Briefly, a DNA construct as illustrated in FIG. 7 (middle) is transformed into wild type Arabidopsis by flower dipping method. T1 seeds are harvested and germinated in soil. 5 day old T1 seedlings are sprayed with LIBERTY® 280SL herbicide to ensure that only transgenic plants survive. The transgenic plants are analyzed by PCR analyses to check the targeted FT site for gene interruption, to determine transgene copy number, and to sequence the targeted insertion site to confirm correct insertion that does not alter the FT gene coding sequence. Events with an HDR insertion of the transgene or indel at the FT target site are grown in growth chambers to observe phenotypes. The majority of transgenic events with randomly integrated transgenes are discarded. Sinceft is a recessive mutation, no late flowering phenotype occurs in hemizygous T1 generation.

T2 seeds are germinated into plants and homozygous transgenic plants identified by qPCR are then grown in growth chambers to evaluate for late flowering phenotype. Some of the plants are sprayed with methoxyfenozide and some are not (controls). The flowering time of sprayed and untreated plants are compared to wt andft plants grown side by side under the same conditions. Typically, Arabidopsis plants flower in ˜4 weeks of initiation in soil under long day (16 hours) photoperiod. Plants showing a delay or absence of flowering in the absence of methoxyfenozide ligand and early or normal flowering in the presence of the ligand are identified and analyzed for EcR switch controlled FT gene expression and flowering.

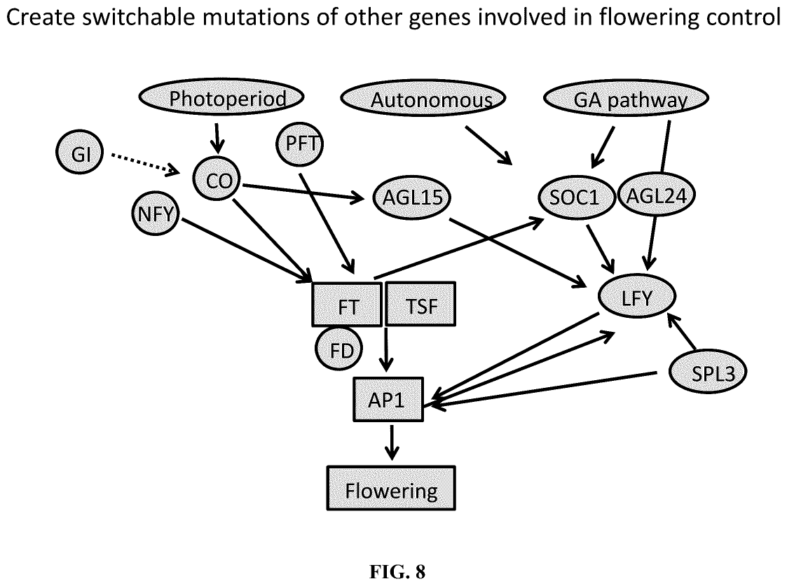

Other flowering genes such as CONSTANS (CO, AT5G15840), FLOWERING LOCUS D (FD, AT3G10390), SUPPRESSOR OF OVEREXPRESSION OF CO 1 (SOC1, AT2G45660), TWIN SISTER OF FT (TSF, AT4G20370), LEAFY (LFY, AT5G61850), and APETALA1 (APi, AT1G69120) upstream or downstream of the major regulatory component FT as illustrated in FIG. 8 can be similarly targeted to control flowering (Ando et al. (2013) Plant Physiol 162:1529-1538; Kimura et al. (2015) Plant Cell Physiol 56:640-649; Yeoh et al. (2011) BMC Biotechnol 11:36, 2011; Yoo et al., (2005) Plant Physiol. 139:770-778).

Similarly, any selected endogenous gene involved in other pathways can be knocked out by inserting a similar switch to create mutants in which the interrupted genes can be turned back on by applying the corresponding ligand.

### Example 5

### Targeting Genes in Plants by Inserting an EcR-Based Gene Switch to Control a Phenotypic Trait.

One could also take advantage of the recent CRISPR gene targeting technology (Li et al. (2015) Plant Physiol. 169:960-970), to gain control over a particular phenotype by targeting an endogenous locus such as the endogenous IMMUTANS gene. This may be targeted to insert an EcR-based gene switch system (e.g., an Activator gene and GAL4 binding motifs) between the IM promoter and its full-length coding sequence. The insertion will likely create an immutans variegation mutant by interrupting the IM gene expression. Simultaneously, the inserted EcR-based gene switch system can be used to turn on the downstream IM gene to restore the mutation. The native IM promoter may be appointed to express the Activator with expression profile mimicking natural expression of the IM gene. In addition, a stronger constitutive promoter, such as 35S may be used to express more EcR-based gene switch Activator proteins to trigger the switch as a positive control.

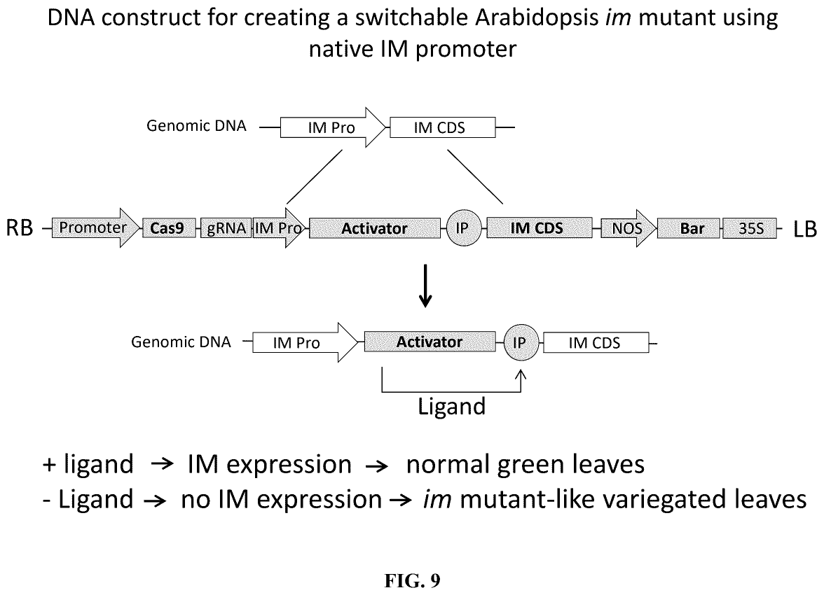

Briefly, DNA constructs as shown in FIG. 9 may be transformed into wild type Arabidopsis. Harvested T1 seeds may be germinated in soil for one week before spraying with LIBERTY® 280SL herbicide to select transgenic seeds. Transformants are analyzed by PCR (PCR) to check the targeted IMMUTANS site for gene interruption, to check transgene copies, and to sequence the targeted insertion site. The majority of transgenic events with randomly integrated transgenes and indels at the target site are discarded. Events with HR-based insertion of the transgene will be further cultivated in growth chambers to observe phenotypes. Since immutans is a recessive mutation, no variegation phenotype is expected in hemizygous T1 generation.

T2 seeds may be germinated in soil for one week before spraying with LIBERTY® 280SL herbicide to select transgenic seedlings. The selected seedlings are further cultivated with or without applications of methoxyfenozide ligand to develop into plants that may display variegation phenotype in the absence of methoxyfenozide and normal green leaves in the presence of methoxyfenozide.

DNA constructs as illustrated in FIG. 10 may similarly be transformed and analyzed as positive controls to show the effect of a stronger constitutive promoter to express Activator proteins sufficient to trigger the EcR-based gene switch system if desired.

### Example 6

### Modify a Plant Trait Through Controlled Expression of a Cytotoxin Gene:

The ribonuclease barnase from Bacillus amyloliquefaciens has been proven an effective transgene for ablating floral tissues in many plant systems. It can be used to block transition to flowering in transgenic plants. To avoid general phytotoxicity, expression of barnase can be restricted to floral progenitor cells, and a number of early floral stage-specific promoters have been identified and validated in Arabidopsis to enable such tissue-specific barnase expression. These promoters are selected for vector construction to drive barnase expression to block flower initiation. There is also another well studied small protein from Bacillus amyloliquefaciens—barstar—which is an extremely potent inhibitor of barnase function. The gene for barstar can be fused to the EcR-based gene switch to achieve controlled expression of barstar by the activator protein which could be expressed under a tissue-specific or constitutive promoter. In the presence of methoxyfenozide ligand inducer, barstar expression is turned on and the resulting barstar protein inhibits barnase ribonuclease function thus restoring the flowering.

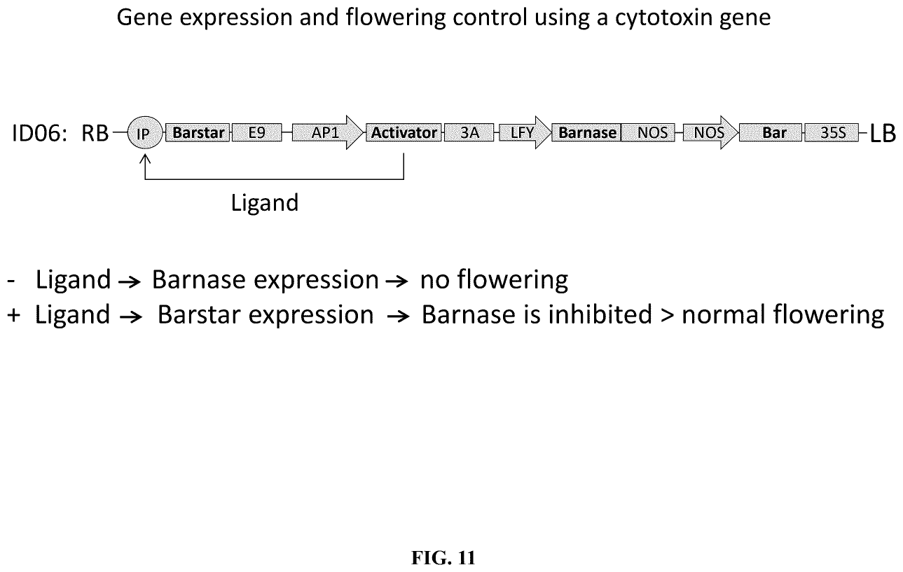

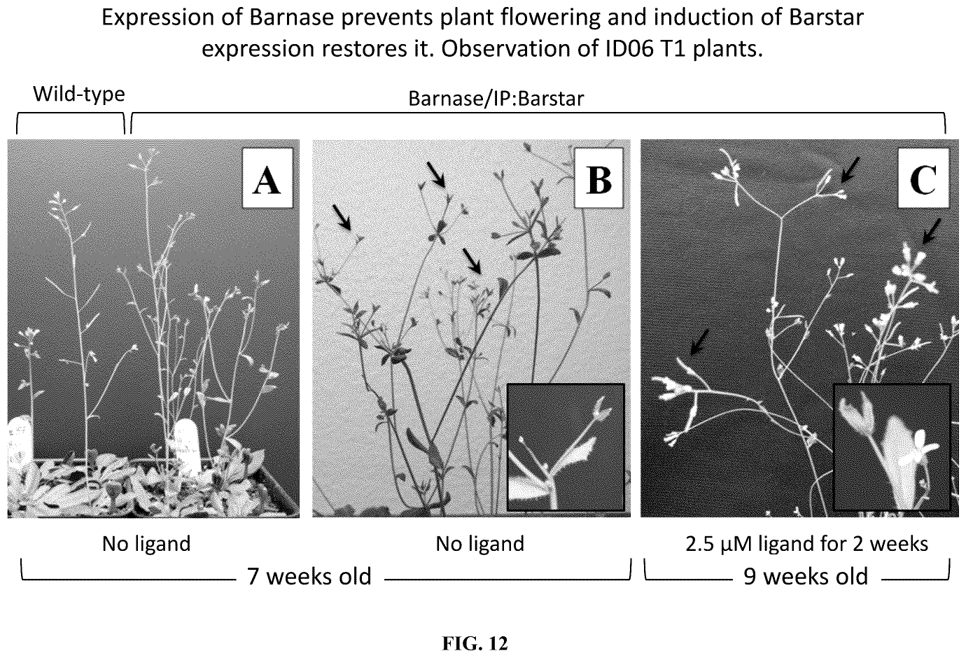

A vector ID06, illustrated diagrammatically in FIG. 11, was constructed to provide constant expression of the barnase ribonuclease from Bacillus amyloliquefaciens in flower primordia to suppress flowering. The DNA construct (ID06) was transformed into wild type Arabidopsis by flower dipping method. T1 seeds were harvested and germinated in soil. Seven day old T1 seedlings were sprayed with LIBERTY® 280SL herbicide and 37 transgenic plants resistant to the herbicide survived. Transformants were analyzed by quantitative PCR (qPCR) to check transgene copy numbers. Thirteen percent of transgenic events contained only one copy of the transgene. All events were grown in growth chambers to evaluate phenotypes. The transgenic plants containing ID06 DNA construct should express the cytotoxic ribonuclease barnase in flower primordia which prevents formation of flowers. Indeed, at 7 weeks of age when wild type Arabidopsis flowered and developed siliques with seeds (FIG. 12A, on the left), the Barnase/IP:Barstar transgenic plants (shown on FIG. 12A, on the right, and on FIG. 12B) did not develop any normal flowers. Instead of flowers, the Barnase/IP:Barstar plants have elongated small leaves that could have derived from sepals as shown in the inset picture of FIG. 12B.

The seven week old non-flowering plants were then sprayed with the ligand methoxyfenozide (2.5 μM) to turn on barstar expression and restore flowering. Ligand applications were performed every other day until all flowers had developed. As early as seven days after ligand application, flower buds and flowers appear as shown in FIG. 12C. T1 plants were grown to maturity and most of them produced siliques and seeds in quantities comparable to wild-type plants. T2 seeds were harvested for further evaluation of the transgenic lines on T2 plants.

Multiple T2 plants from six independent single copy transgenic ID06 events along with wt arabidopsis control plants were grown for three weeks. At three weeks plants from each line were divided in 3 treatment groups of 12 plants in each. The treatment was applied every other day for 30 days. The plants in group A were sprayed with plain water, in group B—with 50 nM solution of methoxyfenozide, and in group C—with 250 nM solution of methoxyfenozide. Before start of the treatment leaf tissue was collected from each plant for DNA extraction and genotype assessment by qPCR to identify homozygous, heterozygous and null segregant plants.

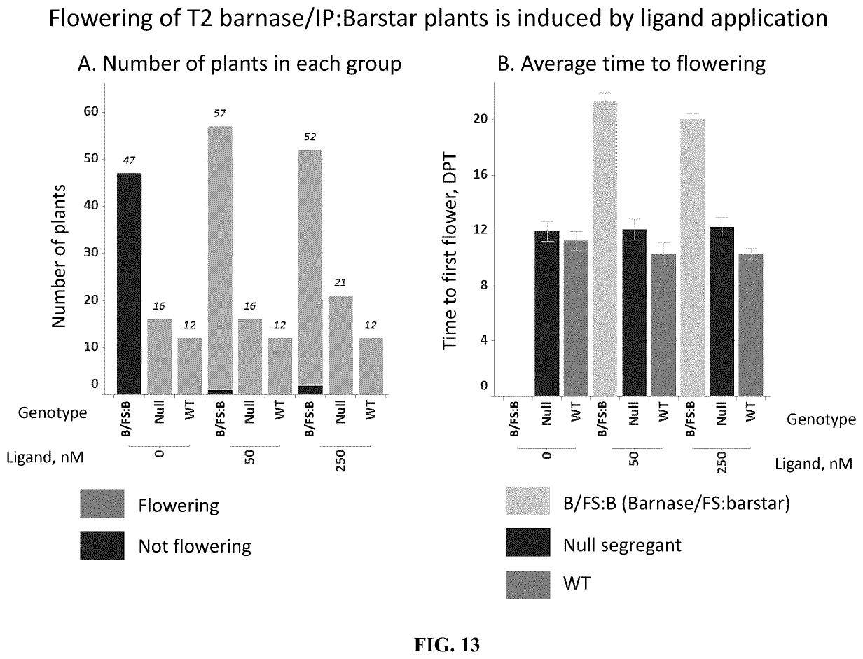

Plants were assessed as to whether they ever flowered (yes/no at the end of experiment, day 51) and the time to start flowering defined as the number of days after the first application of methoxyfenozide (days post-treatment: DPT) until the first flower bud appears. As can be seen on FIG. 13A none of the plants with ID06 transgene construct (labeled B/FS:B genotype) flowered in the absence of inducer ligand (methoxyfenozide), while almost all B/FS:B plants treated with either 50 nM or 250 nM methoxyfenozide developed flowers (56 of 57 plants, and 50 of 52 plants, respectively). All the null segregants and wild-type plants flowered irrespectively of whether or not they were sprayed with methozyfenozide.

There was a 8-10 days delay in the time to start flowering between ligand-treated transgenic (ID06) and non-transgenic (null segregants and wild-type) plants. This delay reflects the timing of expression of activator protein (AP) under APi floral promoter, and the lag in ligand reaching to and interacting with AP. Expressing AP under strong constitutive promoter, e.g., CaMV-35S could shorten the delay.

Methoxyfenozide had the same effect on flowering when applied as either 50 nM or 250 nM solution indicating that minimal effective concentration could be lower than 50 nM.

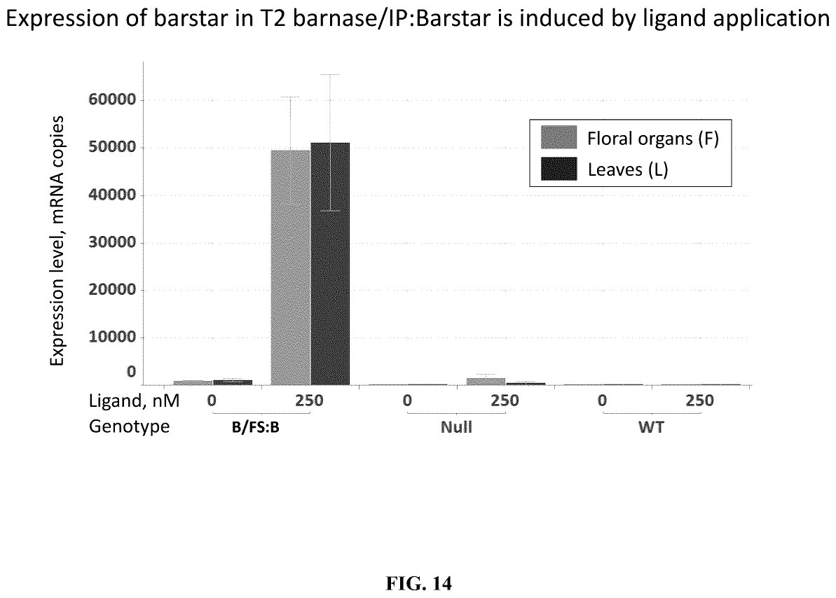

Barnase expression was assessed by qRT-PCR of the leaf and floral tissues sampled from plants treated with 250 nM activating ligand and from plants treated with water. As can be seen on FIG. 14 the expression of barstar is tightly controlled by EcR-based switch in both sampled tissues with expression barely detectable in the absence of ligand and dramatically increased by the ligand.

### Example 7

### Complementation of Late Flowering Through the Ectopic Expression of FT Gene.

Two DNA vectors ID14 and ID16 (described in FIG. 3) were constructed to transform late flowering ft mutant Arabidopsis line CS184 acquired from TAIR, The Arabidopsis Information Resource (at URL address arabidopsis.org). ID14, shown diagrammatically in FIG. 3A, incorporates EcR-based gene switch to control expression of the wt FT transgene by activator ligand, while ID16, shown diagrammatically in FIG. 3B, constitutively overexpresses the wt FT transgene and serves as a positive control for complementation offt mutant phenotype.

Fifty-five T1 transgenic plants with ID14 were obtained. Thirty-two of those flowered in 2 weeks indicating uncontrollable expression of the FT gene resulting in early flowering possibly due to corruption of the inserted construct or an effect of insertion site genomic context that countered EcR-based gene switch control. However, the other twenty-three plants formed large rosette leaves without flowering in 4 weeks implying that the transgenic FT gene was not expressed. For comparison, the ft mutant plants would flower ˜2 weeks later than wild type Arabidopsis plants that flower in ˜4 weeks after planting under similar 16 hours long day conditions. These late flowering ID14 plants would be appropriate to test EcR-based gene switch controlled FT expression. For the ID16 control DNA construct, forty-three T1 transgenic plants were obtained. Forty-one of these flowered prematurely without rosette leaf formation in just 2 weeks after planting. Some plants set seeds in 4 weeks with short siliques. The siliques dried and opened in another ˜2 weeks to expose the seeds inside. Representative photographs taken at 4 weeks after planting are shown in FIG. 4 to show ID14 plants (IP:FT on the left) and ID16 plants (35S:FT on the right).

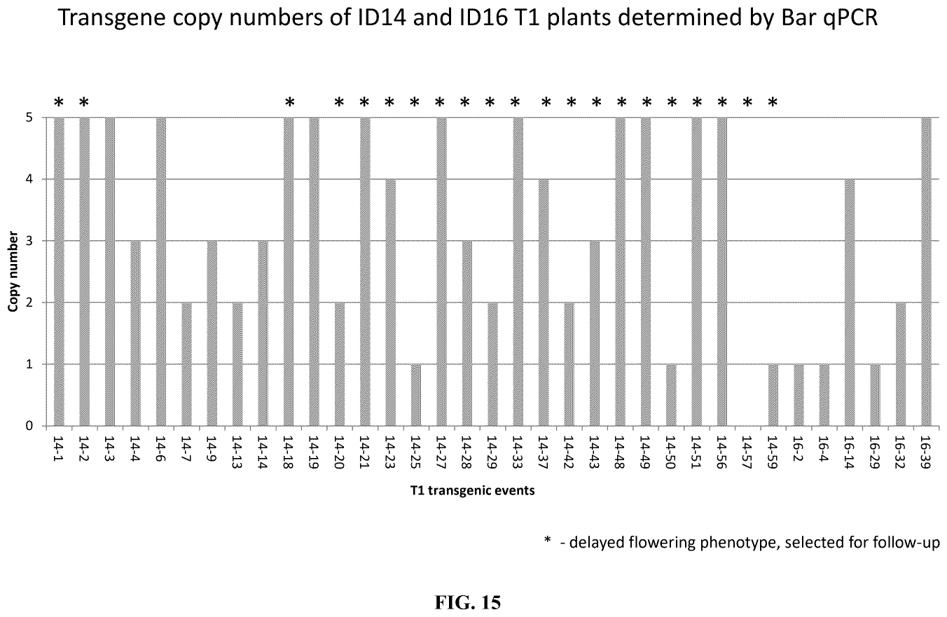

To gain early molecular characteristics of the transgenic events and also to validate some molecular assays we developed for the project, a subset of the ID14 and ID16 transgene T1 plants were sampled, depending on their availability, and preliminarily characterized for copy number by qPCR and FT gene expression by qRT-PCR. To estimate the copy numbers of the transgenic FT gene, TaqMan qPCR assays were designed using the phytoene desaturase gene (PDS) as the endogenous control to normalize the assay and the wild type Arabidopsis genomic DNA as the calibrator. Gene-specific qPCR primers and probes were designed, AtFT-F1/AtFT-R1/AtFT-T1 for FT gene and AtPDS-F1/AtPDS-R1/AtPDS-T1 for PDS gene. Genomic DNA extracted from leaf samples of the transgenic plants were analyzed by multiplex PCR with the above primers using a StepOne plus real time PCR system. Other cleaner transgene-specific qPCR assays such as the 35S-F2/35S-R1/35S-T1 targeting the 35S promoter, and Bar-F1/Bar-R1/Bar-T1 targeting the Bar selectable marker gene of the ID14 and ID16 DNA constructs were designed, validated, and used successfully to confirm the copy numbers of the transgenes obtained by the FT-specific qPCR. As shown in FIG. 15, both the ID14 (IP:FT) and ID16 (35S:FT) plants contained one or more copies of transgenic FT gene as determined by Bar-specific qPCR by using a single copy transgenic event as the calibrator. Events with 5 or more copies of the transgene were all counted as 5 since the qPCR assay was not accurate for high copy numbers. The endogenous 2 alleles of the FT gene were not counted in this assay for the sake of simplicity. Many ID14 plants flowered prematurely except for those indicated by * above the corresponding bars. There was no direct correlation between early flowering and the number of transgenic FT copies.

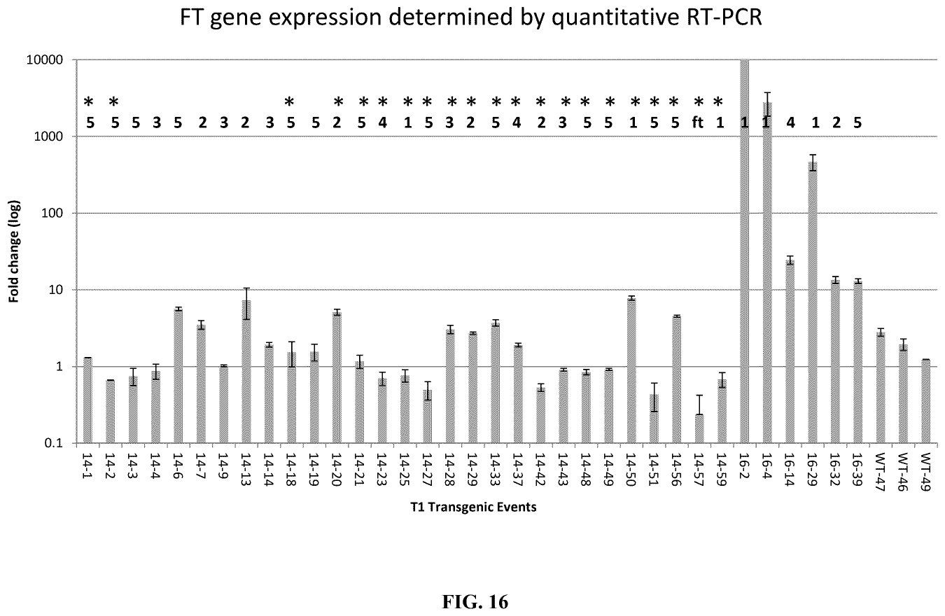

The same subset of transgenic T1 plants was also characterized for FT gene expression in leaves by qRT-PCR as shown in FIG. 16. Total RNA was extracted from samples collected from rosette leave of flowering plants using a Qiagen RNA easy kit, treated with Qiagen DNaseI on column, and checked to be genomic DNA free by a simple PCR assay with primers AtPDS-F2/AtPDS-R2 targeting the endogenous PDS gene. The first cDNA was synthesized from the total RNA to be used as template for qRT-PCR analysis using a qScript cDNA master mix kit (Quanta Biosciences). The cDNA samples were analyzed by the same TaqMan qPCR assays with primers and probes, AtFT-F1/AtFT-R1/AtFT-T1 for the FT target gene and AtPDS-F1/AtPDS-R1/AtPDS-T1 for the endogenous control gene PDS withft mutant Arabidopsis total cDNA as the calibrator. Some ID14 plants that did not flower prematurely (indicated by * above the bars) had similar level of FT expression as some of those plants that did flower prematurely. No positive correlations were found between the expression levels and transgenic FT copy numbers (indicated by the number above the bars). However, it was clear that most of the positive control 35S:FT plants (ID16) which flowered extremely prematurely did have higher levels of FT expression. Since the T1 plants developmental stages were not synchronized and only one time point was sampled due to the limitation of T1 plant materials, this qRT-PCR only served to demonstrate that the 35S:FT transgene in ID16 plants expressed FT gene at levels that were thousand times higher than in wild type or IP:FT (ID14) plants and likely led to extremely early flowering observed in ID16 plants.

It is clear that the late flowering phenotype of Arabidopsis ft mutant can be complemented by the ectopic expression of FT gene, as evidenced by extremely early flowering in 35S:FT plants (ID16) constitutively expressing the FT gene. Early flowering was also observed in some IP:FT plants (ID14) even without ligand application. The low level expression of FT gene in some ID14 plants was probably caused by complex transgene configuration that led to rearrangements and/or by position effect and was apparently sufficient for restoration of flowering in ft mutant. However, many ID14 T1 plants did not flower prematurely. These late flowering ID14 T1 plants were selected for further characterization at T2 generation to evaluate ability of the EcR switch to control flowering through conditioned complementation of ft mutation

### Example 8

Control Flowering Through Controlling the FT Gene Expression with an EcR-Based Gene Switch.

To get an early reading on the possibility of using the EcR-based gene switch to control flowering, a few selected ID14 T1 plants that did not flower within 3 weeks were sprayed with 2.5 μM methoxyfenozide ligand solution every other day for two weeks with aft mutant plant as the control. The treated ID14 plants started bolting within 5 days while the ft mutant plant did not. At day 11, the ID14 plants produced more flowers and pods while theft mutant plant just started bolting. Though very preliminary, the results suggested that the EcR switch responded to the methoxyfenozide ligand induction and turned on FT expression.

T2 seeds were harvested from ID14 T1 plants and several experiments were designed to test if the EcR gene switch is tightly controlling the transgenic FT expression in the absence of the activator ligand, and if the switch can be easily turned on by applying the ligand to express enough FT to induce early flowering. T2 seeds from six independent ID14 lines (three with single copy of the transgene and three—with two copies) were planted along with wild-type Arabidopsis and ft mutant and grown under long day (16 hours) and short day (8 hours) photoperiods. Genotype of these T2 plants: homozygous, heterozygous, and null were assessed by TaqMan qPCR as described in Example 7 with the exception of utilizing only the Bar gene-specific primers and probes Bar-F1/Bar-R1/Bar-T1 and the same PDS endogenous control AtPDS-F1/AtPDS-R1/AtPDS-T1. The T2 plants segregated the transgene as a single gene following the Mendelian inheritance.

The homozygous plants and null segregants that served as isogenic negative controls were subject to treatment with water (as a control) and 2 concentrations of methoxyfenozide: low (250 nM) and high (2.5 μM). Plants were treated in groups of 2 pots with 5 plants in each at short and long day photoperiods. Treatments were administered every other day starting from day 19 (long day) and 25 (short day) after planting and continued for about three weeks until all plants flowered.

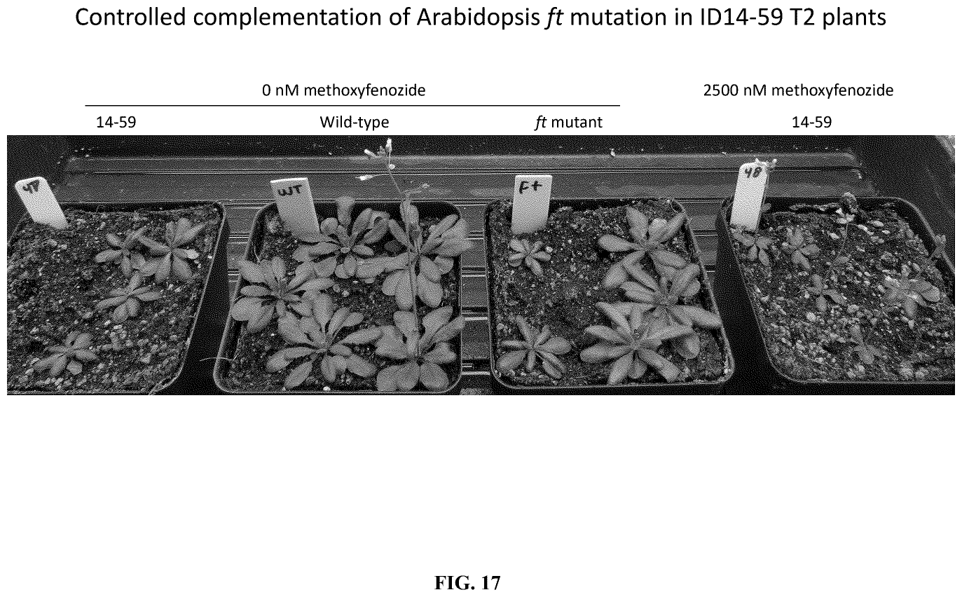

Induction of flowering with methoxyfenozide was clearly observed in most homozygous plants at both concentrations of the ligand. Five days after the first application the ID14 plants started to flower similarly to wild type control plants, while ft mutant and untreated ID14 plants did not flower as shown in FIG. 17. The homozygous T2 plants of one single-copy line (ID14-50) treated with either 250 or 2500 nM methoxyfenozide flowered two to ten days earlier than the wild type and 22 to 30 days earlier than ft mutant plants while the plants mock treated with water flowered at about the same time as the wild type and much earlier than ft mutant plants. As expected, the ID14-50 null segregant plants flowered at the same time as theft mutant plants—around 55 days after planting. Ligand dependent induction of flowering in homozygous ID14 T2 plants was observed under both short day and long day photoperiods. The two-copies ID14 lines showed results similar to single-copy lines.

ID14 T2 plants were maintained to maturity and T3 seeds were collected for further characterization of a selected single-copy line ID14-50. The homozygous and null segregant T3 plants along with Arabidopsis ft mutant plants were grown for three weeks before starting ligand application. Plants were divided in four groups in which up to twelve individual plants per genotype were counted. Each group was treated with different concentrations of methoxyfenozide (10, 50 and, 250 nM) and one group was mock treated (water). Treatments were applied every other day from day 17 (long day) or 24 (short day) for a period of approximately three weeks or until all plants flowered.

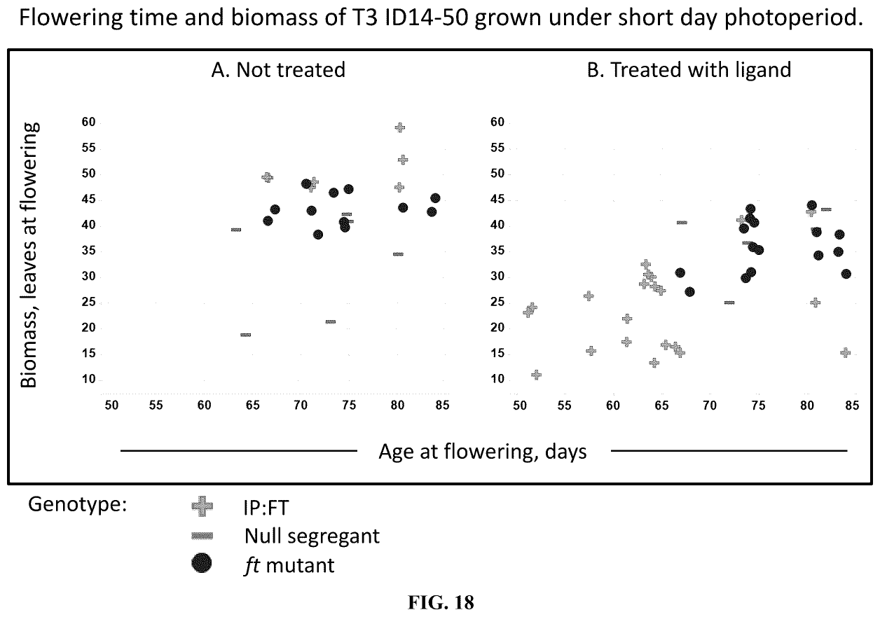

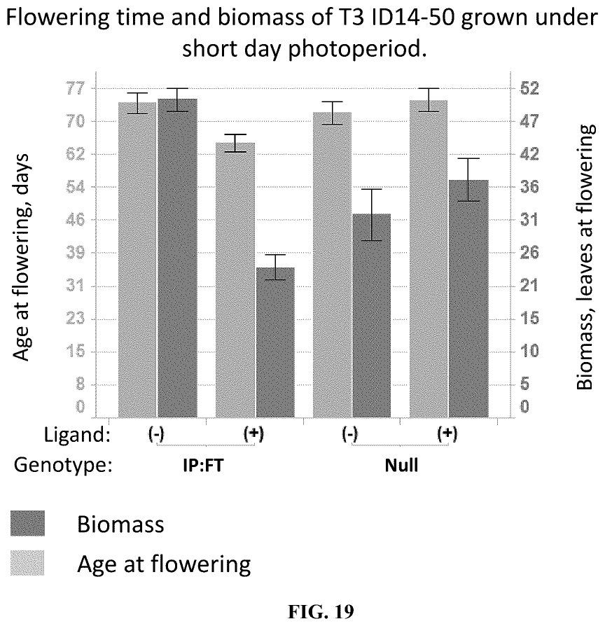

Plants were observed to document the age at the time of flowering (appearance of the first bud) and the number of rosette leaves when the first flower bud appears. The observation was done over a period of ninety days from the day of planting. In both short day and long day photoperiod, the homozygous T3 ID14-50 plants exposed to ligand started flowering as early as fourteen days before any null plants and ft mutants while the untreated plants flowered at a similar time with null and ft mutant plants as shown in FIG. 18. Looking at the number of rosette leaves as a proxy for biomass, only the homozygous T3 ID14-50 plants exposed to all ligand concentrations showed a strong decrease in biomass production at both photoperiods. This observation correlates nicely with early flowering as shown in FIG. 18 and FIG. 19.

FT expression was characterized at five time-points over the time-course of induction. One leaf per plant was collected at time-zero before ligand application corresponding to 17 and 24 days old plants in long and short day photoperiod, at one day after treatment started corresponding to 18 and 25 days old plants, four days after treatment started corresponding to 21 and 28 days old plants, seven days after ligand application started corresponding to 24 and 31 days old plants and, eleven days after treatment started corresponding to 28 and 35 days old plants for RNA extraction using a Qiagen RNA easy kit. First-strand cDNA was synthesized from the total RNA to be used as template for qRT-PCR analysis. qRT-PCR was set-up as previously described for T2 characterization. The homozygous ID14-50 plants showed low FT expression before ligand application which increases as early as one day post-treatment for all concentrations of methoxyfenozide in long day photoperiod and short day photoperiod (data not shown). For most homozygous plants FT expression stayed relatively high and to a similar level from day one to day seven post-treatment before going down a little by day eleven post-treatment. For all time points, tissue was collected at the end of a period of induction, right before treatment was re-applied. This data showed that the level of induction was persistent enough to keep FT level high to the next application of ligand. FT expression in mock-treated plants as well as null and mutant treated plants stayed very low for the most part over the eleven days of treatment. In all genotypes, there is a large variation in FT expression among individual plants in the groups of siblings.

TABLE 1

| | | SEQ |
|---|---|---|
| | | ID |
| Primer | Sequence | NO |
| AtPDS-F1 | CATCTGGAGGTTGTGAACTAATGG | 21 |
| AtPDS-R1 | TTGTTCTTGTCTTAAGCGCTTGA | 22 |
| AtPDS-T1 | 5′ VIC-ACATAGCTTAGGGTTCC-3′ | 23 |
| | MGBNFQ | |
| AtFT-F1 | TTATGGCCAAAGAGAGGTGACTA | 24 |
| AtFT-R1 | CCAATCTCAACTCTTGGCTTGTT | 25 |
| AtFT-T1 | 5′ 6FAM-TGGCTTGGATCTAAGGCCTTCTCA- | 26 |
| | ZEN-3′IBFQ | |
| 35S-F2T | GATGTGATATCTCCACTGACGT | 27 |
| 35S-R1 | CGTGTCCTCTCCAAATGAAATGA | 28 |
| 35S-T1 | 5′ 6FAM-CGCACAATCCCACTATCCTTCGCA- | 29 |
| | ZEN-3′IBFQ | |
| Bar-F1 | GAGGTCGTCCGTCCACTC | 30 |
| Bar-R1 | GTCAACCACTACATCGAGACAAG | 31 |
| Bar-T1 | 5′ 6FAM-CGGTTCCTGCGGCTCGGTAC- | 32 |
| | ZEN-3′IBFQ | |
| AtPDS-F2 | TGTTTGGGAATGTTTCTGCGG | 33 |
| AtPDS-R2 | CTTCAAGCAACAGAGGTTTGTG | 34 |

### Example 9

Transformation of Petunia to Downregulate the Chalcone Synthase Gene and Expression on Demand: Plants were generated to express interference RNA (RNAi) to downregulate the petunia chalcone synthase A (ChsA) gene (amino acid sequence SEQ ID NO:56). Dowregulation of the ChsA gene decreases the number of intact ChsA transcripts in the plant cells and thereby decreases anthocyanin production which resulted in a change in flower color from purple to white.

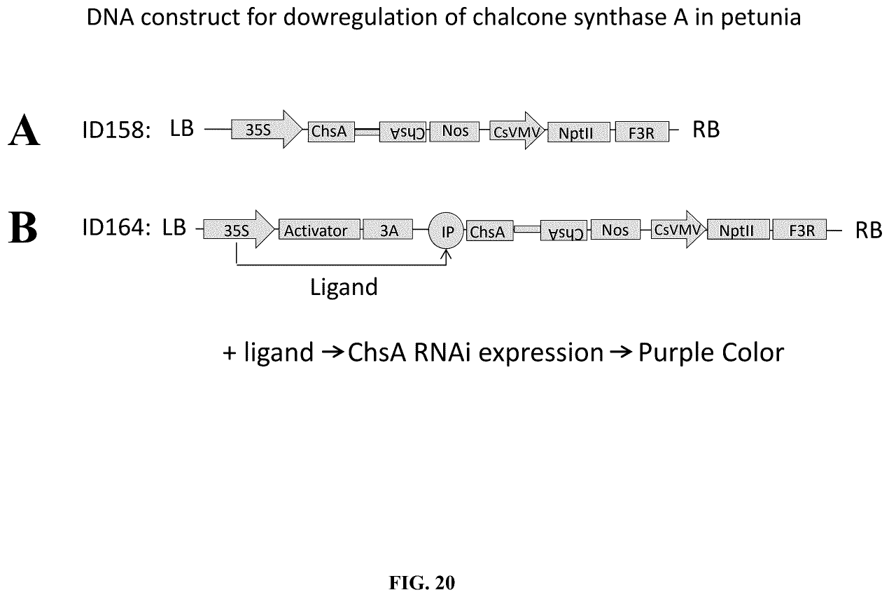

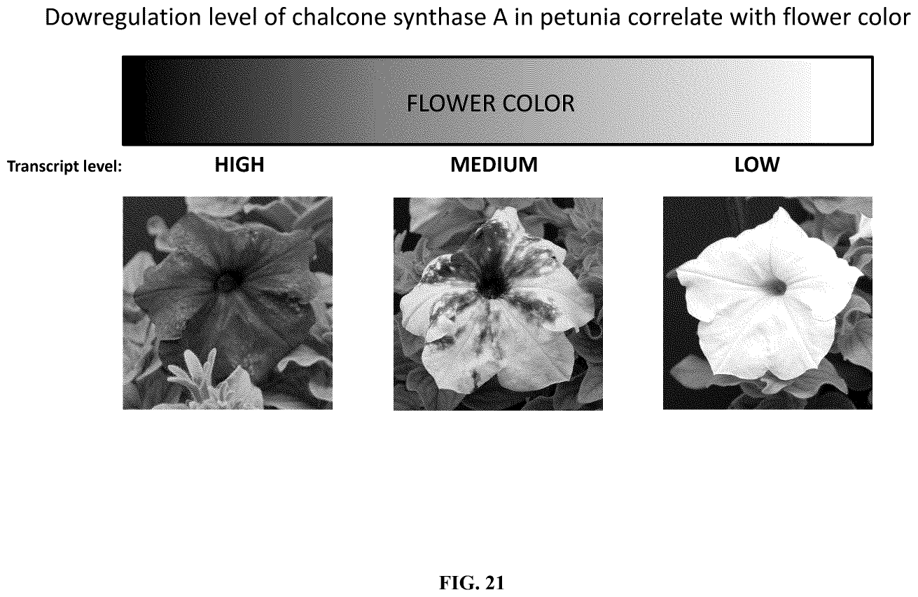

DNA construct ID158 (SEQ ID NO:59) (LB-35S:ChsA RNAi+CsVMV:NptII-RB) as shown in FIG. 20A was used as a positive control to transform wild-type Petunia hybrida variety V26. A 35S-CaMV promoter is driving the expression of a ChsA hairpin transcript (SEQ ID NO:64) (half hairpin is shown in SEQ ID NO:63) promoting targeted transcript degradation through gene silencing. The targeted region of ChsA is SEQ ID NO:62. CsVMV:NptII is used as the selectable marker gene for selecting transgenic plants with kanamycin antibiotic. To estimate the copy number of the transgene in TO transgenic plants, TaqMan qPCR assays were designed using the phytoene desaturase gene PDS as the endogenous control and a pre-characterized transgenic Petunia genomic DNA as the calibrator. Gene-specific qPCR primers and probes were designed, NptII-F1/NptII-R1/NptII-T1 for NptII gene and PhPDS-F1/PhPDS-R1/PhPDS-T1 for PDS gene. Genomic DNA extracted from leaf samples of the transgenic plants were analyzed by multiplex PCR with the above primers using a StepOne plus real time PCR system. Forty plants containing low copies of the transgene (two copies and below) were chosen to analyze correlation between ChsA transcript level and flower color. Total RNA was extracted from leaf samples of these 40 plants using a Qiagen RNA easy kit and checked to be genomic DNA free by a simple PCR assay targeting the endogenous PDS gene (PhPDS-F2/PhPDS-R2). The first cDNA was synthesized from the total RNA to be used as template for qRT-PCR analysis. qPCR primers and probes, PhChsA-F1/PhChsA-R1/PhChsA-T1 for the ChsA target gene and PhPDS-F1/PhPDS-R1/PhPDS-T1 for the endogenous control gene PDS were used with wild type Petunia total cDNAs as the calibrator. Plants containing ID158 DNA showed an array of flower color correlating with the level of ChsA transcript present in the plant cell as shown in FIG. 21. A wild-type plant was fixed to a ChsA transcript level of one. Most plants showing level of ChsA expression from one to 0.3 was dark purple color. Flowers from plants with ChsA transcript level from 0.3 to 0.06 were mostly variegated and plants with a really low ChsA transcript level (0.06 and below) were having predominantly white flowers. There were few biological exceptions showing a high level of ChsA transcripts with white flowers and vice versa.

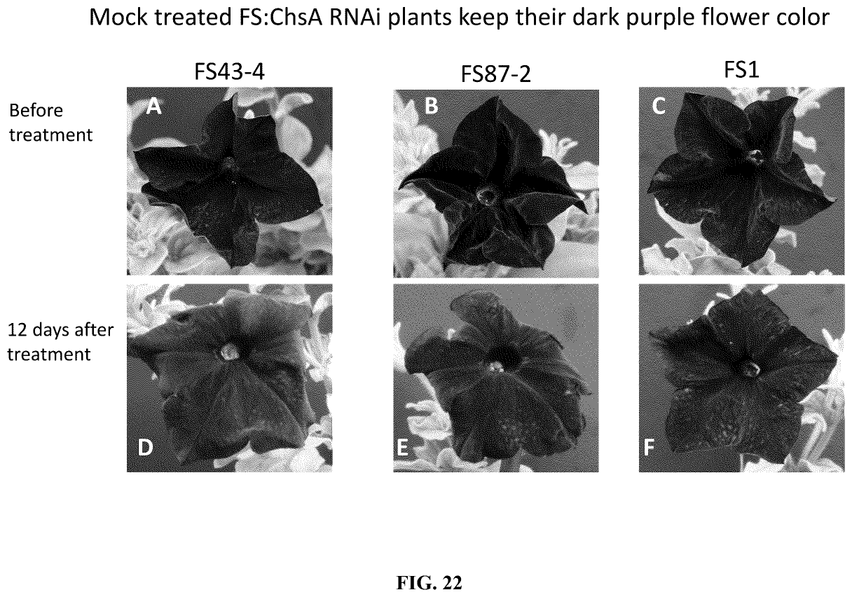

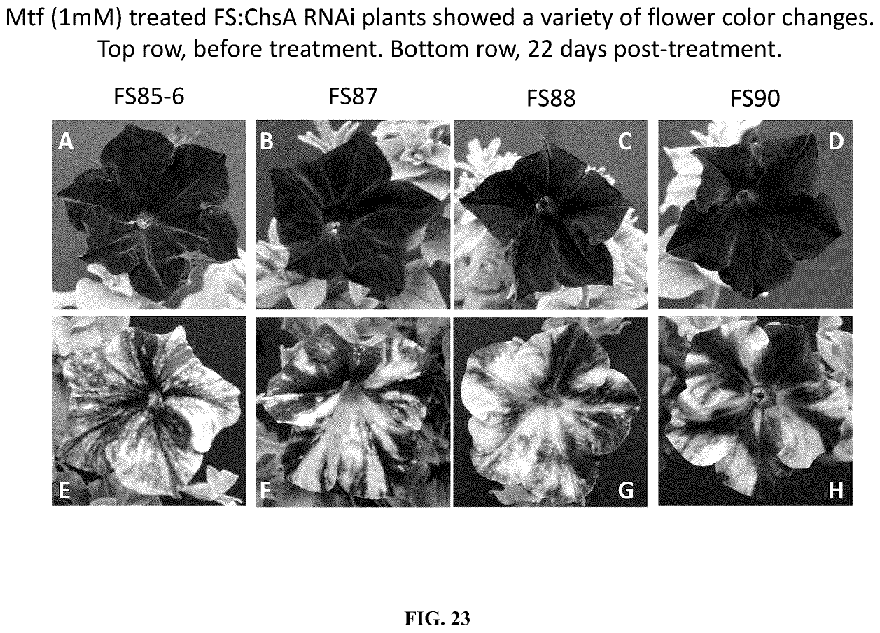

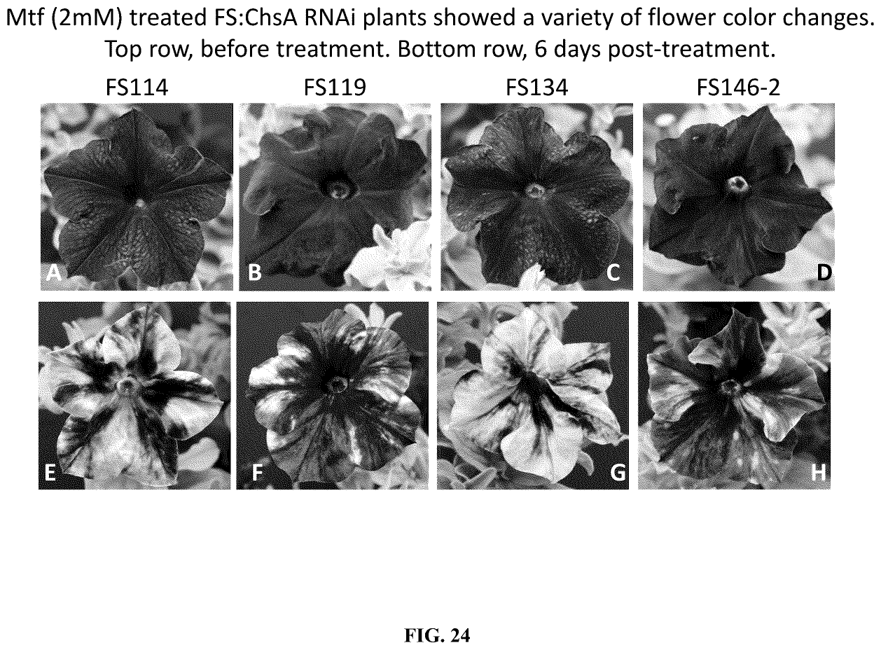

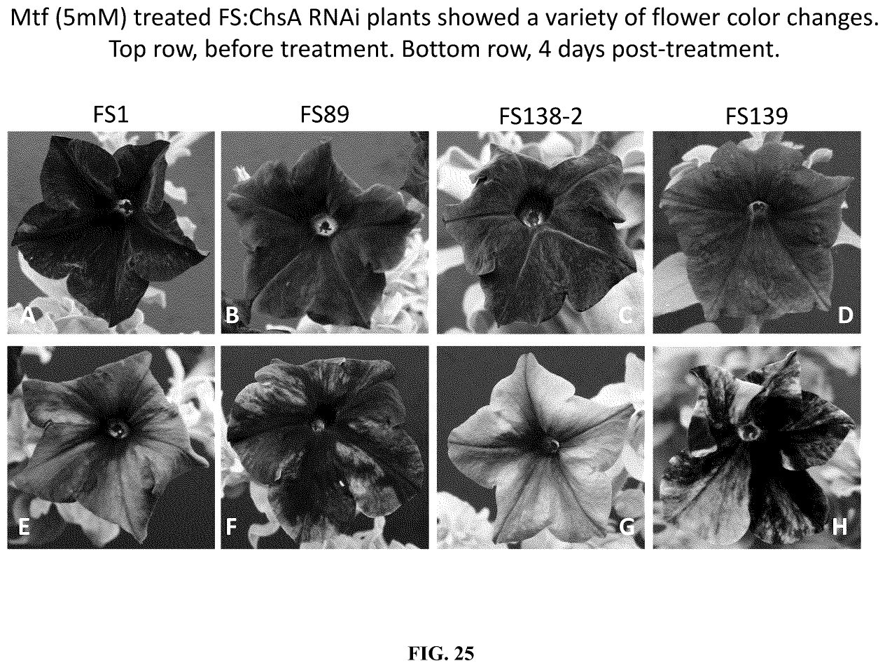

DNA construct ID164 (LB-IP:ChsA RNAi+35S:AP+CsVMV:NptII-RB) (SEQ ID NO:55) containing both the inducible promoter (IP) composed of 5 GAL4 regulatory elements (GAL4-RE) placed upstream of the minimal CaMV-35S promoter and the multidomain activator (AP) protein gene (VGE(E68V/V184I/Y204E) under the control of CaMV-35S promoter is shown in FIG. 20B. ID164 (SEQ ID NO:55) was used to transform wild type (wt) Petunia hybrid variety V26. The chalcone synthase hairpin transcript will not be express until the activator ligand (methoxyfenozide; Mtf) is provided to mobilize the activator proteins for binding to the GAL4-RE and switching the inducible promoter FS on. Transgene copy number was assessed for 56 TO transgenic plants by Tagman assay. 84% were low copies (two copies and below), 12.5% were high copy (three copies and above) and, few plants were nulls. Before ligand application most (98%) plants were flowering as dark purple color. Plants were divided in five groups, each group being treated differently. Treatment occurred every other day at a rate of 30 ml per plant. Treatment is sprayed on plants (leaves and flowers). Water is used as a mock treatment while INTREPID® 2F insecticide (Mtf being the active ingredient) is used as a ligand treatment. Group one was mock treated. Group two was treated with 2.5 uM Mtf in INTREPID® 2F insecticide form. Group three was treated with 1 mM Mtf (highest recommended dose on ornamental plants for INTREPID® 2F). Group four was treated with 2 mM Mtf and group five treated with 5 mM Mtf. All plants mock treated keep their original flower color as seen in FIG. 22. However, plants treated with Mtf showed diverse flower color change depending on ligand concentration as shown in FIG. 23. Few plants treated with 2.5 uM (50%) showed flower color change from dark purple to light purple 10 days post-treatment. When increasing the ligand concentration to 1 mM, change in flower color, dark purple to variegate, was observed faster (7 days post-treatment) on 50% of plants as shown in FIG. 23. With 2 mM and 5 mM ligand, one application (2 days post-treatment) was sufficient to change flower color from dark purple to variegate as shown in FIG. 24 and FIG. 25, respectively. Decrease in ChsA gene expression level in treated plants compared to their higher level before ligand treatment correlates with their change in flower color as shown in FIG. 21.

### Example 10

Assay for Agro-infiltration. Transient β-glucoronidase (GUS) expression, was modified from previously reported literature (Wroblewski et al. (2005) Plant Biotechnol. J. 3(2):259-273). Specifically, Agrobacterium tumefaciens strain EHA105 containing an appropriate plasmid for GUS expression was grown overnight to an OD600 of 0.8 and re-suspended in induction buffer with the following components: MS basal medium (Sigma-Aldrich), 10 mM MgCl2, and 100 uM acetosyringone. After 4 hours, induced culture suspension was infiltrated into detached fully expanded petunia leaves (approximately 0.1 mL/leaf). Leaves were incubated in a humidity chamber at 28° C. for 3 days.

Histochemical X-gluc staining for GUS visualization. Leaf discs from Agro-infiltrated petunia leaves were submerged in GUS assay buffer: 10 mM EDTA (pH=8), 100 mM NaH2PO4, 0.01% Triton X-100, 2 mM X-gluc (5-bromo-4-chloro-3-indolyl-β-D-glucuronic acid, cyclohexylammonium salt). Leaf discs were placed under vacuum for 5 minutes and then incubated at 37° C. for 24 hours. Leaf discs were then submerged in 95% ethanol and incubated at 37° C. for 24 hours to remove chlorophyll for easier visualization of X-gluc staining.

Botrytis cinerea isolation and inoculation. The plant pathogen was isolated from diseased strawberries displaying typical symptomology. A single spore colony was isolated using standard plant pathology methodologies and the ITSI—5.8S rDNA—ITSII locus was amplified and sequenced to validate its identity. The isolate used is 100% identical to more than 20 previously reported B. cinerea isolates in Genbank. For plant inoculation B. cinerea was grown on V8 Agar media under fluorescent light for 2 weeks until the typical grey mass of aerial spores was visible. A section of the colony was placed into 10 mL of sterile DI water and vortexed vigorously to dislodge spores. The resulting solution was filtered through a 70 micron mesh to yield a homogenous spore solution free of mycelia and agar media. The spore solution was spun down and washed 3× in sterile DI water and quantified using a hemocytometer. The solution was diluted in sterile DI water to a final concentration of 200,000 spores/ml. For petunia inoculations, detached leaves were wounded with a sterile forceps (approximately 0.5 mm abrasion) and the wounded site was inoculated with 10 μL of spore solution (2,000 spores/inoculation site). Three leaves/treatment and three wound sites/leaf were inoculated with spore solution for each assay.

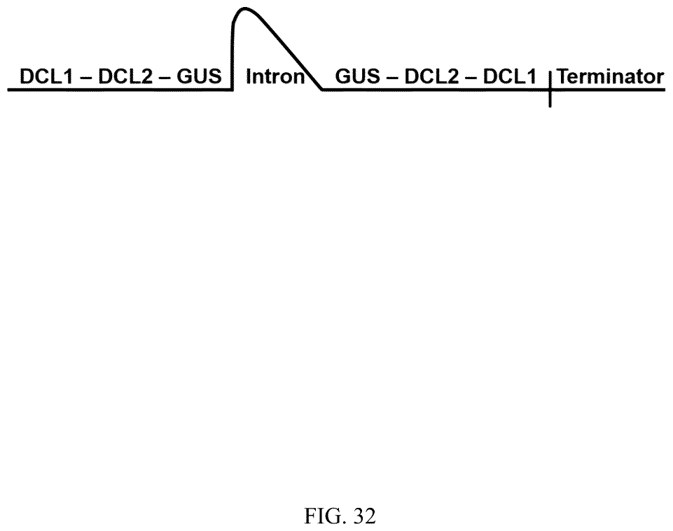

A Dicer-like Glucuronidase hybrid hairpin (DCL1/DCL2/GUS Hairpin) (SEQ ID NO:35) construct was developed for transgenic-based disease resistance in Petunias as shown in FIG. 32.

Cassettes for Agrobacterium Mediated Transformation. Two cassettes for Agrobacterium-mediated transformation of petunia were generated for constitutive and ligand inducible Botrytis cinerea resistance.
A. (LB-CsVMV:NPTII+35S: DCL1/DCL2/GUS hairpin-RB): Constitutive dsRNA expression (ID157)(SEQ ID NO:60).
B. (LB-CsVMV:NPTII+35S:Activator+IP:DCL1/DCL2/GUS hairpin-RB): Switch-controlled dsRNA expression (ID163)(SEQ ID NO:57)
LB=Left Border
NPTII=Kanamycin Resistance gene IP=Inducible Promoter
DCL1=Botrytis cinerea (Bc) Dicer Like Protein 1 partial
DCL2=Be Dicer Like Protein 2 partial
GUS=β-glucoronidase (reporter gene) (encoding amino acid sequence SEQ ID NO:58), partial
RB=Right Border

Once may also generate cassettes for switch-controlled expression of anti-pest peptides. For example, an antifungal polypeptide with activity against Botrytis cinerea could be constructed with the antifungal peptide (AFP):
C. (LB-CsVMV:NPTII+35S:AFP-RB): constitutive AFP expression
D. (LB-CsVMV:NPTII+35S:Activator+IP:AFP-RB): Switch-controlled AFP Expression LB=Left Border
   NPTII=Kanamycin Resistance gene
   IP=Inducible Promoter
   AFP=Botrytis cinerea antifungal peptide
   RB=Right Border

Petunia Transformation and Regeneration. Petunia variety V26 was transformed with the constructs A and B using Agrobacterium tumefaciens and regenerated using Kanamycin selection according to previously described protocols and standardized methodologies. The same strategy may also be used to transform Petunias with constructs C and D.

Event Characterizations. All event characterization for A and B was carried out using TO plants. All plants were screened for the presence of the selectable NPTII marker and copy number within the genome was determined using QPCR.

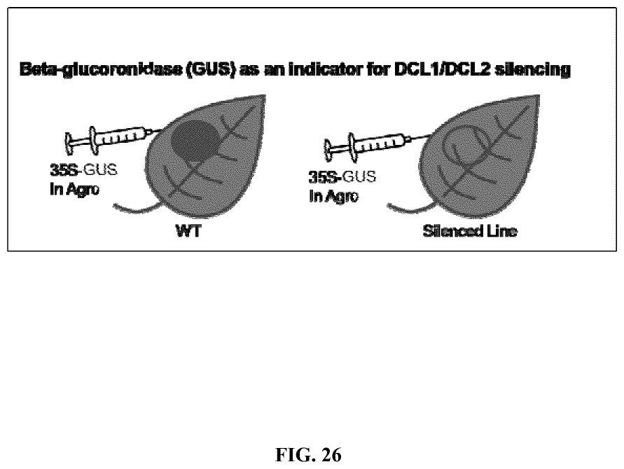

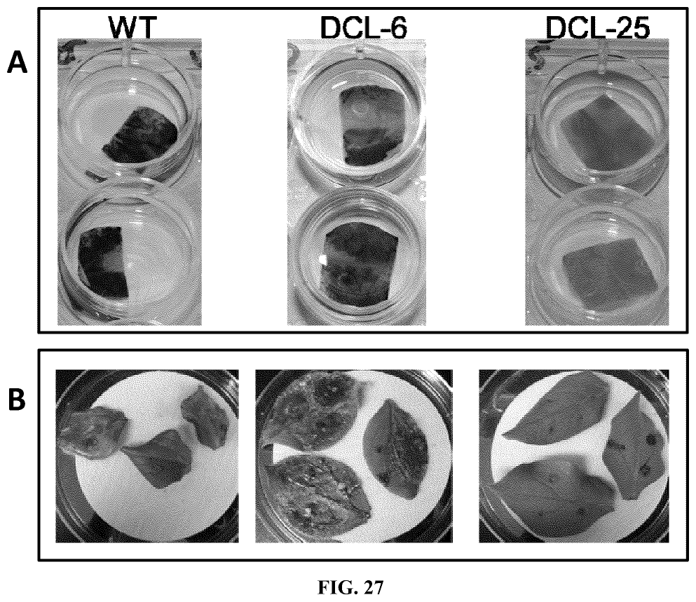

Constitutive hairpin expression for dsRNA synthesis targeting 2 Botrytis cinerea genes and the GUS reporter gene. A strategy for validating dsRNA expression through silencing of the reporter gene β-glucoronidase (GUS) was adapted from previous literature (Wroblewski et al. (2007) Plant J. 51:803-818). In our construct, the GUS fragment was fused to the previously-validated DCL1/DCL2 fragments for dsRNA expression. Silencing of GUS in transgenic Petunia lines was evaluated by transient Agrobacterium tumefaciens mediated expression through leaf infiltration as shown diagrammatically in FIG. 26. All lines were infiltrated with Agrobacterium containing a constitutively expressed GUS gene. Three days post-infiltration GUS expression was visualized using the histochemical X-gluc stain. Successful expression of the hairpin cassette would be expected to result in a lack of blue X-gluc staining as shown in FIG. 26. Multiple petunia transformation events were screened by infiltrating detached leaves with Agrobacterium and performing X-gluc staining. Selected lines were identified that blocked transient GUS expression (e.g., DCL-25) while other lines did not block GUS expression (e.g., DCL-6), presumably linked to hairpin expression activity. The results are shown in FIG. 27A. This colormetric screening technique is important for evaluating a HIGS hairpin cassette expression, as there is no endogenous plant gene targeted for down regulation that can be empirically measured to identify plant lines with successful dsRNA expression. Inoculation of the selected petunia lines with Botrytis cinerea spore solution showed a strong correlation between GUS silencing and disease resistance. As shown in FIG. 27B, there was a strong correlation between GUS silencing and disease resistance indicating both phenotypes are linked to initiation of RNAi through expression of the hairpin cassette shown above.

Constitutive expression of AFP. Transgenic petunia events transformed with the constitutively expressed an AFP cassette are screened by quantitative reverse-transcriptase PCR (qrtPCR) using the protocol as described in Example 7 and AFP specific primers are designed to determine the level of expression among independent lines.

When inoculated with Botrytis cinerea spores, AFP lines are expected to show disease resistance that should correlate to AFP expression levels while independent GFP transformed petunia lines should all be susceptible to B. cinerea.

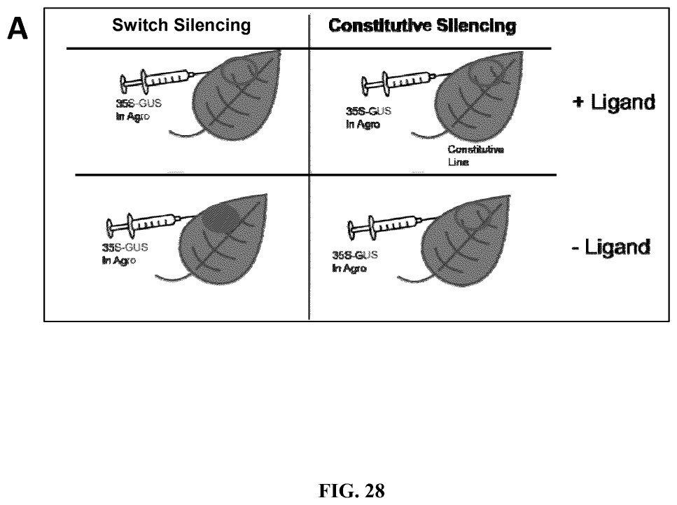

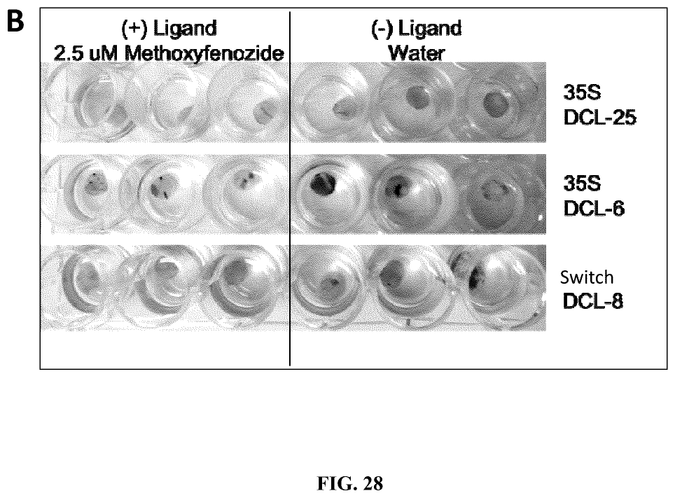

Ligand inducible hairpin expression for dsRNA synthesis targeting two Botrytis cinerea genes and the GUS reporter gene. Ligand inducible expression of the DCL1/DCL2/GUS hairpin cassette was evaluated using the transient Agrobacterium-mediated GUS expression as described above only detached leaves were sprayed with 50 uM ligand or mock treated with water prior to Agro-infiltration. As shown in the cartoon on FIG. 28A, under ligand inducible hairpin expression we expected to see GUS silencing only following ligand application.

Eight independent Switch-DCL1/DCL2/GUS petunia lines were evaluated by detaching 6 leaves from each line and splitting into two treatments. One treatment was sprayed with 50 uM ligand while the other treatment was sprayed with water. At 2 days after the initial treatment, plants were treated again with 50 uM ligand or water. At 3 days after the initial treatment, plants were Agro-infiltrated in order to transiently express GUS. At 3 days after Agro-infiltration, plants were histochemical stained with X-gluc for visualization of GUS expression. Constitutive controls DCL-25 and DCL-6 analyzed in FIG. 27 did not show any ligand induced changes to GUS expression, while a single Switch DCL-8 line shown in FIG. 28B showed ligand induced GUS silencing as shown by the absence of blue staining.

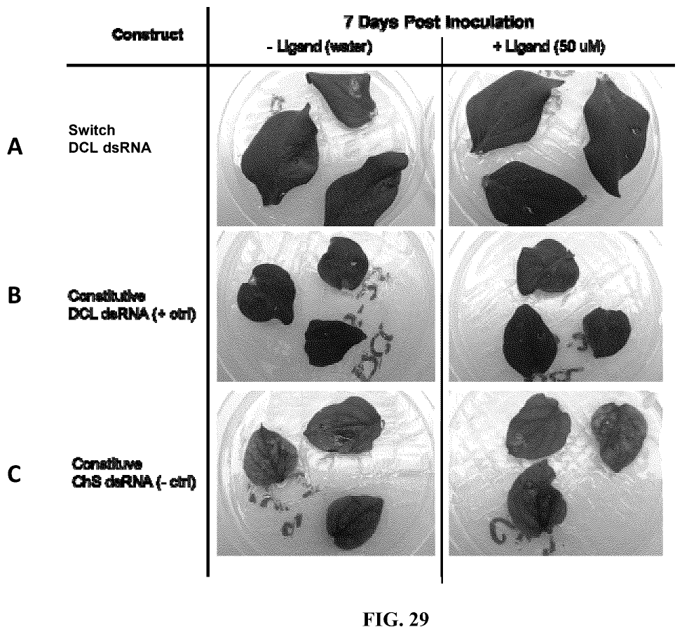

For ligand inducible disease resistance screening, 6 leaves from each line were split into two treatments. One treatment was placed on 50 uM methoxyfenozide in 1% phytoagar (+Ligand) while the other treatment was placed on 1% phytoagar (−ligand). After 3 days all leaves were inoculated with Botrytis cinerea spore solution. As shown in FIG. 29, at 7 days after pathogen inoculation, the Switch-DCL1/DCL2/GUS hairpin line showed ligand induced disease resistance. The constitutive DCL1/DCL2/GUS hairpin line DCL-25 (+control) and non-specific dsRNA line (−control) showed no change in disease resistance in the presence or absence of ligand.

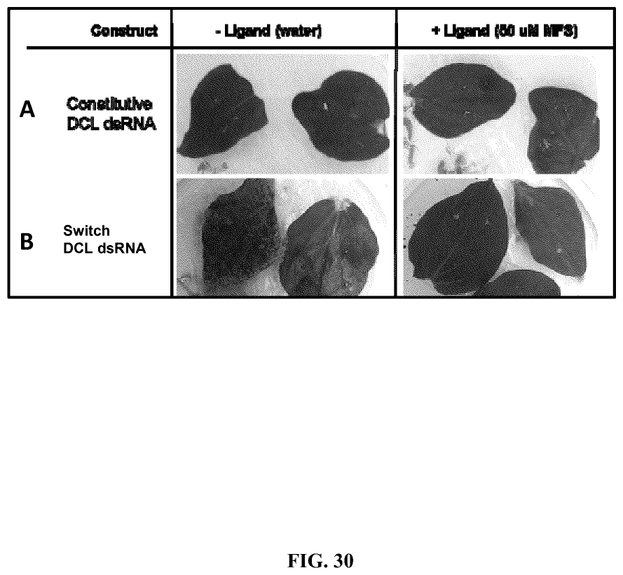

A composite of switch-coupled DCL constructs with ligand and in the absence of ligand is shown in FIG. 30.

### Example 11

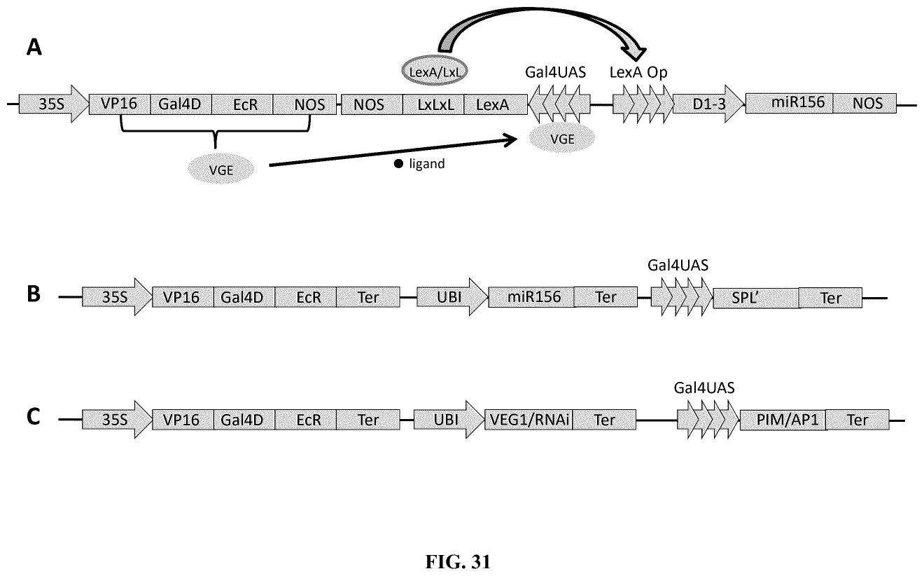

FIG. 31 shows three constructs that illustrate a strategy to increase biomass in plants. In FIG. 31A, a construct is shown to allow constitutive expression of miR156 (SEQ ID NO:61), an miRNA that effectively silences the SPL flowering gene. The construct also contains a gene switch with a repression domain (LxLxL) in place of the activation domain such that when ligand is present, miR156 is repressed by the expression of LexA/with its LxLxL domain and flowering occurs (Wang, J-W (2009) Cell 138:738-749). Thus, in this example, one may grow the plants to allow increased biomass by inhibition of flowering until flowering is desired. Thereupon, application of the chemical ligand of the gene switch represses the miR156 inhibition and flowering is restored.

FIG. 31B shows a construct that allows constitutive expression of miR156, however, in this arrangement, in the presence of ligand, the switch leads to expression of SPL′ which is a modified SPL flowering gene with nucleotide changes (which encode the same amino acid sequence) that make it resistant to miR156 silencing, thereby leading to flowing in the presence of ligand through expression of the engineered SPL′. Thus, in this example, one may grow the plants to allow increased biomass by inhibition of flowering until flowering is desired. Application of the chemical ligand at the appropriate time allows the gene switch-controlled SPL′ gene to be expressed and flowering is restored.

In FIG. 31C, the construct allows constitutive expression of Veg1/RNAi which silences the VEG1 flowering gene, but in the presence of ligand, the gene switch leads to expression of PIM (AP1) and restores flowering. Thus, in this example, one may grow the plants to allow increased biomass by inhibition of flowering until flowering is desired. Thereupon, application of the chemical ligand of the gene switch overrides VEG1 inhibition and flowering is restored.

1. One or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a nucleic acid which controls a trait or traits in a plant; and,
(c) a regulatory element operably connected 5′ of b), wherein said EcR-based gene switch induces expression of b) in the presence of a chemical ligand.

2. The one or more vectors or polynucleotides of claim 1 wherein said traits or traits comprises one or more of flowering, drought resistance, herbicide resistance, pest resistance, diseases resistance, type of floral color, intensity of floral color, floral aroma, accumulation of specific nutrients, taste, aroma, color, taste, nutritional value, or expression of an exogenous gene.

3. The one or more vectors or polynucleotides of claim 2, wherein said nucleic acid encodes a protein wherein said protein has a toxic effect on said plant or imposes a metabolic burden on said plant during expression.

4. One or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a nucleic acid which controls flowering in plants; and,
(c) a regulatory element 5′ of b), wherein said EcR-based gene switch induces expression of b) in the presence of a chemical ligand.

5. The one or more vectors or polynucleotides of claim 4 wherein said nucleic acid that controls flowering comprises a polynucleotide encoding a full-length polypeptide, or biologically active fragment thereof, of one or more of Gigantea (GI), Nuclear Factor Y (NFY), Constans (CO); protein farnesyltransferase (PFT), Agamous-Like 15 (AGL15), Flowering Locus (FT); Twin Sister of FT (TSF), bZIP transcription factor (FD), Flowering Locus T, Apetala1 (AP1), Suppressor of Overexpression of CO1 (SOC1), Agamous-Like 24 (AGL24); Leafy (LFY), and Squamosa Promoter Binding Protein-Like 3a (SPL3).

6. The one or more vectors or polynucleotides of claim 4 wherein said nucleic acid that controls flowering is Flowering Locus (FT).

7. One or more vectors or polynucleotides comprising:
a) a polynucleotide encoding an ecdysone receptor-based (EcR-based) gene switch or an ecdysone receptor-based (EcR-based) gene switch comprising a VGEvy ligand binding domain;
b) an immutans variegation mutant (IM) nucleic acid; and,
c) a regulatory element 5′ of b), wherein said EcR-based gene switch induces expression of b) in the presence of a chemical ligand.

8. One or more vectors or polynucleotides comprising:
a) a polynucleotide encoding an ecdysone receptor-based (EcR-based) gene switch or an ecdysone receptor-based (EcR-based) gene switch comprising a VGEvy ligand binding domain;
b) a nucleic acid encoding a protein or an RNA that inhibits a plant pathogen; and,
c) a regulatory element 5′ of b), wherein said EcR-based gene switch induces expression of b) in the presence of a chemical ligand.

9. The one or more vectors or polynucleotides of claim 8 wherein said protein or RNA that inhibits a plant pathogen is a defensin family protein.

10. The one or more vectors or polynucleotides of claim 9 wherein said defensin family protein comprises one or more of Defensins, Snakins, Heveins, Thionins, Lipid Transfer Proteins, Cyclotides, Shepherins, MBP-1, Vicilin-like peptides, Impatiens family peptides, Beta-Barrelin or Knottins family proteins.

11. The one or more vectors or polynucleotides of claim 9 wherein said pathogen is a virus, a fungus, a bacterium, or an insect.

12. The one or more vectors or polynucleotides of claim 11 wherein said virus comprises one or more of Tobacco mosaic virus, Tomato spotted wilt virus, Tomato yellow leaf curl virus, Cucumber mosaic virus, Potato virus Y, Cauliflower mosaic virus, African cassava mosaic virus, Plum pox virus, Brome mosaic virus, Potato virus X, Citrus tristeza virus, Barley yellow dwarf virus, Potato leafroll virus, or Tomato bushy stunt virus.

13. The one or more vectors or polynucleotides of claim 11 wherein said fungus comprises one or more of Alternaria, Botrytis, Fusarium, Pyricularia, Verticillium, Aspergillus, Saccharomyces, Trichophyton, Cercospora, Cladosporium, Leptosphaeria, Penicillium, Trichoderma, Septoria; Plectosphaerella, Colletotrichum, Bipolaris, Ascochyta, Phytophthora, Gibberella, Mycosphaerella, Neurospora, Phoma, Pythium, Rhizoctonia, Helinthosporium, Geotrichum, Sclerotinia, Clavibacter, Pyrenopkora, Nectria; Candida; Cryptococcus, Ceratocystis, Chalara, or Venturia.

14. The one or more vectors or polynucleotides of claim 13 wherein said fungus is Botrytis cinerea.

15. The one or more vectors or polynucleotides of claim 11 wherein said bacterium comprises one or more of Bacillus, Enterococcus, Staphylococcus, Clavibacter, Ralstonia, Listeria, Rhizobium, Erwinia, Escherichia, Pseudomonas, Agrobacterium, Sarcina, Ralstonia, Micrococcus, Proteus, Klebsiella, Salmonella, Serratia Streptococcus, or Xanthomonas.

16. The one or more vectors or polynucleotides of claim 15 wherein said bacterium is Erwinia amylovora.

17. The one or more vectors or polynucleotides of claim 11 wherein said insect is a Spodoptera or Helicoverpa.

18. The one or more vectors or polynucleotides of claim 17 wherein said insect is Spodoptera frugiperda.

19. The one or more vectors or polynucleotides of claim 8 wherein said RNA is an RNA molecule having a first portion comprising a first nucleotide sequence that has sufficient homology to a second nucleotide sequence on a second portion so said first portion and said second portion hybridize to form a double-stranded RNA (dsRNA).

20. One or more vectors or polynucleotides comprising nucleic acids encoding:
(a) an ecdysone receptor-based (EcR-based) gene switch;
(b) a Bacillus amyloliquefaciens Barstar protein;
(c) a Bacillus amyloliquefaciens barnase ribonuclease; and,
(d) a regulatory element 5′ of (b), wherein said EcR-based gene switch induces expression of b) in the presence of a chemical ligand.

21. The one or more vectors or polynucleotides of claim 20 wherein said nucleic acid encoding barnase ribonuclease is operatively linked to a plant promoter of a plant gene of interest to suppress expression of said plant gene of interest.

22. The one or more vectors or polynucleotides of claim 21 wherein said promoter is a promoter related to flowering.

23. The one or more vectors or polynucleotides in any one of claims 20 to 22, wherein said EcR-based gene switch comprises a ligand binding domain derived from Choristoneura fumiferana (Cf).

24. The one or more vectors or polynucleotides in any one of the preceding claims, wherein said EcR-based gene switch further comprises a heterologous (non-Cf) DNA-binding domain and a heterologous (non-Cf) transcriptional transactivation domain.

25. The one or more vectors or polynucleotides of claim 24, wherein said DNA-binding domain comprises one or more of a Gal4 binding domain, a LexA binding domain, a transcription factor binding domain, a Group H nuclear receptor member binding domain, a steroid/thyroid hormone nuclear receptor superfamily member binding domain, a bacterial LacZ binding domain, an EcR binding domain, an ARF, a bZIP, a homeodomain family of a DNA binding domain, or synthetic DNA-binding protein.

26. The one or more vectors or polynucleotides of any of the preceding claims, wherein said EcR-based gene switch comprises a transactivation domain comprising one or more of a Group H nuclear receptor member transactivation domain, steroid/thyroid hormone nuclear receptor transactivation domain, synthetic or chimeric transactivation domain, polyglutamine transactivation domain, basic or acidic amino acid transactivation domain, a VP16 transactivation domain, a GAL4 transactivation domain, an NF-κB transactivation domain, a BP64 transactivation domain, a B42 acidic transactivation domain (B42AD), a p53 transactivation domain, a p65 transactivation domain (p65AD), an EDLL acidic transactivation domain from AP2/ERF, a glutamine-rich transactivation domain, an ARFs or CONSTANS protein transactivation domain, a proline-rich domain transactivation domain from bZIP or an analog, combination, or modification thereof.

27. The one or more vectors or polynucleotides of claim 24, wherein said DNA-binding domain is a GAL4 transcription factor DNA-binding domain and/or wherein said transcriptional transactivation domain is derived from a Sarcoma Virus 40 (SV40) or comprises a herpes virus VP16 transcriptional transactivation domain.

28. The one or more vectors or polynucleotides of claim 23, wherein said CfEcR ligand binding domain comprises one or more amino acid substitutions compared to a wild-type (wt) CfEcR ligand binding domain.

29. The one or more vectors or polynucleotides of claim 28, wherein said CfEcR ligand binding domain comprises the polypeptide sequence of SEQ ID NO:2.

30. The one or more vectors or polynucleotides of any one of the preceding claims, wherein said EcR-based gene switch comprises the polypeptide sequence of SEQ ID NO:1.

31. The one or more vectors or polynucleotides in any one of the preceding claims, wherein said chemical ligand is a non-steroidal chemical ligand.

32. The one or more vectors or polynucleotides of claim 31, wherein said chemical ligand is a diacylhydrazine.

33. The one or more vectors or polynucleotides of claim 32, wherein said chemical ligand is methoxyfenozide.

34. The one or more vectors or polynucleotides of claim 1 wherein said regulatory element comprises a recognition site for a DNA binding protein portion of said EcR-based gene switch and a promoter.

35. The one or more vectors or polynucleotides of any one of the preceding claims further comprising a nucleic acid sequence encoding a second gene switch and a second gene of interest wherein said second gene of interest is operatively linked to a second regulatory element operatively linked 5′ of said second gene of interest wherein said second gene switch binds said second regulatory element in the presence of a second chemical ligand.

36. The one or more vectors or polynucleotides of 35 wherein said second gene switch comprises any of an ecdysone receptor (EcR) based gene switch, a rapamycin-based gene switch, a prokaryotic tetracycline repressor-based gene switch, a lactose repressor-operator-based gene switch, a cumate-based gene switch, or a biotin-based gene switch.

37. The one or more vectors or polynucleotides of claim 36 wherein said second chemical ligand is the same or different chemical ligand that is active for said EcR-based gene switch.

38. The one or more vectors or polynucleotides of any of the preceding claims, wherein said nucleic acid further encodes a polypeptide comprising a repression domain comprising any of an AUX/IAA protein with an LxLxL domain, or an ERF-associated amphiphilic repression (EAR) domain.

39. A plant comprising the one or more vectors or polynucleotides of any one of the preceding claims.

40. The plant of claim 39, wherein all or a portion of the one or more vectors or polynucleotides is integrated into the plant genome.

41. The plant of claim 39 or 40, wherein said plant is a dicot or a monocot.

42. A method of reducing pollen drift from genetically engineered plants to wild plant species wherein the one or more vectors or polynucleotides of claims 1 to 6 are introduced to said genetically engineered plants to control, inhibit or prevent cross-pollination of wild plant species.

43. The method of claim 42, wherein said genetically engineered plants are forage crops or turfgrass including alfalfa, barley, buckwheat, bulgur wheat, corn, durum wheat, einkorn, emmer, farro, fonio, kamut, millet, oats, rice, rye, semolina wheat, sorghum, spelt, teff, triticale, wheat, bamboo shoots, barleygrass, lemongrass, molasses, rapadura, sugarcane, wheatgrass, Amaranth, Coxcomb, pitseed goosefoot, quinoa, chia, acacia seed, wattleseed, Kentucky bluegrass, perennial ryegrass, tall fescue, fine fescue, creeping bentgrass, creeping red fescue, hard fescue, chewings fescue, Bermudagrass, buffalograss, kikuyugrass, St. Augustine, or zoysia.

44. A method of improving crop yield comprising introduction of the one or more vectors or polynucleotides of claims 1 to 38 into crop plants.

45. The method of claim 44, wherein the crop plants are fruits or vegetables.

46. The method of claim 45, wherein the fruits or vegetables are alfalfa sprouts, apples, apricots, artichokes, Asian pears, asparagus, atemoyas, avocados, bamboo shoots, bananas, beans, bean sprouts, beets, belgian endive, bitter melons, bell peppers, blackberries, blueberries, bok choy, boniato, boysenberries, broccoflowers, broccoli, broccolini, brussels sprouts, butter lettuce, cabbage, cantaloupe, carambola, carrots, casaba melons, cauliflower, celery, chayotes, cherimoyas, cherries, coconuts, coffee, collard greens, corn, cranberries, cucumbers, dates, eggplant, endive, escarole, feijoa, fennel, figs, garlic, gooseberries, grapefruit, grapes, green beans, green onions, collard greens, mustard greens, guava, hominy, honeydew melons, horned melons, iceberg lettuce, Jerusalem artichokes, jincama, kale, kiwifruit, kohlrabi, kumquats, leeks, lemons, lettuce, lima beans, limes, longan, loquats, lychees, madarins, malangas, mandarin marijuana, oranges, mangos, mulberries, mushrooms, napas, nectarines, okra, onions, oranges, papayas, parsnip, passion fruits, paw-paws, peaches, peanut, pears, sugar snap peas, green peas, peppers, persimmons, pineapples, plantains, plums, pomegranates, potatoes, prickly pears, pummelos, pumpkins, quince, radicchio, radishes, raspberries, red cabbage, rhubarb, romaine lettuce, rutabaga, shallots, snow peas, soybeans, spinach, sprouts, squash, strawberries, string beans, sweet potatoes, tangelo, tangerines, tomatillo, tomatoes, turnip, ugli fruit, watermelons, water chestnuts, watercress, waxed beans, yams, yellow squash, yuca/cassava, or zucchini squash.

47. A method of generating plants with inducible resistance to pathogens comprising introduction of the one or more vectors or polynucleotides of any one of claims 8 to 18 into said plants.

48. A method of generating plants with inducible resistance to biological stressors comprising introduction of the one or more vectors or polynucleotides of any one of claims 1 to 3 into said plants.

49. The method of claim 48, wherein the biological stressors are drought or herbicides.

50. A method of generating control of flowering in fruits, vegetables or other plant produce to aid in harvest timing, comprising introduction of the one or more vectors or polynucleotides of any one of claims 1 to 6 into plants generating said fruits, vegetables or plant produce.

51. The method of claim 50, wherein the fruits and vegetables are, alfalfa sprouts, apples, apricots, artichokes, Asian pears, asparagus, atemoyas, avocados, bamboo shoots, bananas, beans, bean sprouts, beets, belgian endive, bitter melons, bell peppers, blackberries, blueberries, bok choy, boniato, boysenberries, broccoflowers, broccoli, broccolini, brussels sprouts, butter lettuce, cabbage, cantaloupe, carambola, carrots, casaba melons, cauliflower, celery, chayotes, cherimoyas, cherries, coconuts, coffee, collard greens, corn, cranberries, cucumbers, dates, eggplant, endive, escarole, feijoa, fennel, figs, garlic, gooseberries, grapefruit, grapes, green beans, green onions, collard greens, mustard greens, guava, hominy, honeydew melons, horned melons, iceberg lettuce, Jerusalem artichokes, jincama, kale, kiwifruit, kohlrabi, kumquats, leeks, lemons, lettuce, lima beans, limes, longan, loquats, lychees, madarins, malangas, mandarin marijuana, oranges, mangos, mulberries, mushrooms, napas, nectarines, okra, onions, oranges, papayas, parsnip, passion fruits, paw-paws, peaches, peanut, pears, sugar snap peas, green peas, peppers, persimmons, pineapples, plantains, plums, pomegranates, potatoes, prickly pears, pummelos, pumpkins, quince, radicchio, radishes, raspberries, red cabbage, rhubarb, romaine lettuce, rutabaga, shallots, snow peas, soybeans, spinach, sprouts, squash, strawberries, string beans, sweet potatoes, tangelo, tangerines, tomatillo, tomatoes, turnip, ugli fruit, watermelons, water chestnuts, watercress, waxed beans, yams, yellow squash, yuca/cassava, or zucchini squash.

52. A method of controlling flowering in plants wherein commercial value is derived from the flower, comprising introduction of the one or more vectors or polynucleotides of any one of claims 1 to 6 into said plants.

53. A method of controlling expression of a plant a gene of interest in a plant comprising introducing the one or more vectors or polynucleotides of any one of claims 1 to 38 into said plant.

54. A method of increasing plant-based production of active pharmaceutical ingredients, comprising introducing the one or more vectors or polynucleotides of any one of claims 1 to 3 into said plant.

55. A method of generating increased efficiency or effectiveness in methods of commercial seed production, comprising introduction of one or more vectors or polynucleotides of the invention into said plants.

56. A method of controlling when a crop plant flowers, to produce greater biomass production, comprising introduction of the one or more vectors or polynucleotides of any one of claims 1 to 6 into said crop plant.

57. A method of controlling timing of fruiting and seed production in plants, comprising introduction of the one or more vectors or polynucleotides of any one of claims 1 to 6 into said plants.

58. A method of controlling or regulating flowering in plants comprising introduction of the one or more vectors or polynucleotides of any one of claims 1 to 6 into said plants.

59. The method of any one of claims 42 to 58, further comprising contacting said plants with a non-steroidal chemical ligand.

60. The method of claim 59, wherein said chemical ligand is a diacylhydrazine.

61. The method of claim 60, wherein said chemical ligand is methoxyfenozide.

62. The one or more vectors or polynucleotides of claims 1 to 7 wherein said plant is African daisy, Agapanthus, Ageratum houstonianum, Alchemilla, Allium, Alyssum, Amaranthus, Amaryllis, Anemone, Angelonia, Anthurium, Artemisia, Asclepias syriaca, Aster, Astilbe, Astrantia, Aubreita deltoidea, baby's breath, bachelor button, balloon flower, bee balm, begonia, bellflower, blanketflower, Bergenia, black-eyed Susan, blanket flower, blazing star, bleeding heart, bluebell, blue-eyed grass, blue star flower, Bouvardia, Bougainvillea, broom, Buddleja, bush morning glory, buttercup, butterfly weed, butterfly bush, Calendula, California poppy, calla lily, Calliandra, Camellia, Campenula, candytuft, canna lily, cape primrose, cardinal flower, carnation, catmint, celosia, chrysanthemum, Clarkia, clover, clematis, cockscomb, columbine, coneflower, coral bells, Coreopsis, Cosmos, Cotoneaster, Crocus, creeping phlox, Crocosmia, crown imperial, cuckoo flower, Cyclamen, Dahlia, day lily, Delphinium, Echium, English bluebell, Erigeron, evening primrose, Euphorbia, flannel flower, flax flower, floss flower, forget-me-not, Forsythia, foxglove, frangipani, freesia, fuschia, gardenia, geranium, gas plant, Gaura, gayfeather, Gerbera, Gladiolus, globeflower, goldenrod, grape hyacinth, Gypsophila, heather, Hebe, Helenium, Heliotrope, Hellebore, hibiscus, hollyhock, honeysuckle, hosta, hyacinth, hydrangea, Hypericum, hardy geranium, hybrid tea roses, Iceland poppy, ice plant, Ilex, Impatiens, Ipheion uniflorum, iris, Ixia, Ixora, Jaborosa, Jacob's ladder, Jamesia americana, jasmine, Jupiter's beard, kaffir lily, Kalmia, kangaroo paw, Kerria, Knautia macedonica, Kniphofia, Kolkwitzia, lady's slipper, Lamium, Lantana, larkspur, Lavatera, lavender, Lechenaultia, lilac, lily, lily of the valley, Linaria, lisianthus, lobelia, loosestrife, lotus, lunaria, lupin, magnolia, Maltese cross, Mandevilla, Marguerite daisy, marigold, Matthiola, mayflower, Meconopsis, mimosa, Mina lobate, mock orange, monk's hood, moonflower, morning glory, Muscari, narcissus, nasturtiums, Nemesia, Nemophila, Nerine, New Guinea impatien, Nicotiana, Nierembergia, Nigella, Nolana, oleander, orchid, oriental lily, oriental poppy, Osteospermum, oyster plant, ox eye daisy, painted daisy, pansy, passion flower, peace lily, Pelargonium, Penstemon, peony, Persian buttercup, Peruvian lily, petunia, pincushion flower, pink lady's slipper, pointsettia, Polyanthus, poppy anemone, Portulaca grandiflora, Primula, Quaker ladies, Queen Anne's lace, Queen's cup, Queen of the meadow, quince, rain lily, Ranunculus, Rhododendron, rock rose, Rondeletia, rose, rose of Sharon, Salvia splendens, Saponaria, Scabiosa, Scaevola, scented geranium, Scilla, Sedum, shasta daisy, shrub roses, Silene, silver lace vine, snapdragon, snowball bush, snowdrop, snowflake, statice, strawflower, sun drop, sunflower, sweet pea, Syringa, tea rose, tiger flower, tiger lily, Tithonia, Trillium, Triteleia, Tritonia crocata, trumpet vine, tuberose, tulip, urn plant, Ursinia, Uva ursi, Verbena, Veronica incana, Vinca, Viola tri-colour, Violet, Virginia creeper, wallflower, wandflower, water lily, Watsonia, wax plant, Wedelia, Weigela, wild rose, wild violet, winter aconite, winterberry, winter jasmine, wishbone flower, wisteria, wooly violet, Xerophyllum, Xylobium, Xylosma, yarrow, yellow angel, yellow bell, yellow-eyed grass, yellowhorn, Zenobia, or zinnia.

63. One or more vectors or polynucleotides comprising:
(a) a polynucleotide encoding an ecdysone receptor-based (EcR-based) gene switch or an ecdysone receptor-based (EcR-based) gene switch comprising a VGEvy ligand binding domain;
(b) a nucleic acid which downregulates trait or traits in a plant; and,
(c) a regulatory element operably connected to a repressor element, wherein said EcR-based gene switch induces expression of said repressor element in the presence of a chemical ligand and said repressor element represses (b).

64. The one or more vectors or polynucleotides of claim 63 wherein said repressor element is a LexA
